SEQUENCE LISTING (55 sequences)

SEQ ID NO: 1 - DNA, 360 bp, synthetic construct
  source 1..360: mol_type = other DNA
caggtgcaactgcagcagcctggggctgaagtggtgaagcctggggcttcagtgagactgtcctgcaagacttctggctacaccttcaccagcaactggatgcactgggtgaagcagaggcctggacaaggccttgagtggatcggagagattgatccttctgatagttatactaactacaatcaaaagttcaagggcaaggccaagttgactgtagacaaatcctccagcacagcctacatggaagtcagcagcctgacatctgaggactctgcggtctattactgtgcaagaggtagcaacccttactactatgctatggactactggggtcaaggaacctcagtcaccgtctcctca

SEQ ID NO: 2 - DNA, 360 bp, synthetic construct
  source 1..360: mol_type = other DNA
caggtgcaactgcagcagcctggggctgaagtggtgaagcctggggcttcagtgagactgtcctgcgagacttctggctacaccttcaccagcaactggatgcactgggtgaagcagaggcctgaccaaggacttgagtggatcggagagattgatccttctgatagttatactaactacaatcaaaagttcaagggcaaggccgaattgactgtagacaaatcctccagcacagcctacatggaagtcagcgacctgacatctgaggactctgcggtctattactgtgcaagaggtagcaacccttactactatgctatggactactggggtcaaggaacctcagtcaccgtctcctca

SEQ ID NO: 3 - DNA, 360 bp, synthetic construct
  source 1..360: mol_type = other DNA
caggtgcaactgcagcagcctggggctgaagtggtgaagcctggggcttcagtgagactgtcctgcaagacttctggctacaccttcaccagcaactggatgcactgggtgaagcagagacctggacaaggacttgagtggatcggagagattgatccttctgatagttatactaactacaatcaaaagttcaagggcaaggccgaattgactgtagacaaatcctccagcacagcctacatggaagtcagcgacctgacatctgaggactctgcggtctattactgtgcaagaggtagcaacccttactactatgctatggactactggggtcaaggaacctcagtcaccgtctcctca

SEQ ID NO: 4 - DNA, 360 bp, synthetic construct
  source 1..360: mol_type = other DNA
caggtgcaactggagcagcctggggctgaagtgaagaagcctggggcttcagtgaaggtgtcctgcaaggcttctggctacaccttcaccagcaactggatgcactgggtgaagcagaggcctggacaaggacttgagtggatcggagagattgatccttctgatagttatactaactacaatcaaaagttcaagggcaaggccaagttgactgtagacaaatcctccagcacagcctacatggaagtcagcgacctgacagctgaggactctgcggtctattactgtgcaagaggtagcaacccttactactatgctatggactactggggtcaaggaacctcagtcaccgtctcctca

SEQ ID NO: 5 - DNA, 360 bp, synthetic construct
  source 1..360: mol_type = other DNA
caggtgcaactggagcagcctggggctgaagtggtgaagcctggggcttcagtgaaggtgtcctgcaagacttctggctacaccttcaccagcaactggatgcactgggtgaagcagacgcctggaaaaggacttgagtggatcggagagattgatccttctgatagttatactaactacaatcaaaagttcgatggcaaggccaagttgactgtagacaaatcctccagcacagcctacatggaagtcagcgacctgacagctgaggactctgcgacctattactgtgcaagaggtagcaacccttactactatgctatggactactggggtcaaggaacctcagtcaccgtctcctca

SEQ ID NO: 6 - DNA, 360 bp, synthetic construct
  source 1..360: mol_type = other DNA
caggtgcaactggagcagcctggggctgaagtgaagaagcctggggcttcagtgaaggtgtcctgcaaggcttctggctacaccttcaccagcaactggatgcactgggtgagacaggcacctggaaaaggacttgagtggatcggagagattgatccttctgatagttatactaactacaatcaaaagttcgatggcaaggccaagttgactgtagacaaatcctccagcacagcctacatggaagtcagcgacctgacatctgaggactctgcgacctattactgtgcaagaggtagcaacccttactactatgctatggactactggggtcaaggaacctcagtcaccgtctcctca

SEQ ID NO: 7 - DNA, 360 bp, synthetic construct
  source 1..360: mol_type = other DNA
caggtgcaactggagcagcctggggctgaagtggtgaagcctggggcttcagtgaaggtgtcctgcaagacttctggctacaccttcaccagcaactggatgcactgggtgaagcagaggcctggacaaggacttgagtggatcggagagattgatccttctgatagttatactaactacaatcaaaagttcgatggcaaggccaagttgactgtagacaaatcctccagcacagcctacatggaagtcagcgacctgacatctgaggactctgcgacctattactgtgcaagaggtagcaacccttactactatgctatggactactggggtcaaggaacctcagtcaccgtctcctca

SEQ ID NO: 8 - DNA, 311 bp, synthetic construct
  source 1..311: mol_type = other DNA
caaattgttctcacccagtctccagcaatcatgtctgcatctccaggggagaaggtcaccatgacctgcagtgccagctcaggtgtcaactacatgcactggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 9 - DNA, 311 bp, synthetic construct
  source 1..311: mol_type = other DNA
caaattgctctcacccaggagccagcaatcatgtctgcatctccaggggagaaggtcaccatgacctgcagtgccagctcaggtgtcaactacatgcactggtatcagcagaagccaggcacctcccccaaaagatggatttatgacacatccaaactggattctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 10 - DNA, 311 bp, synthetic construct
  source 1..311: mol_type = other DNA
gacattgttctcacccagtctccagcaactttgtctgcatctccaggggagaaggtcaccatgacctgtagtgccagctcaggtgtcaactacatgcactggtatcagcagaagccaggcacctcccccaaaagatggatttatgacacatccaaactggattctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 11 - DNA, 311 bp, synthetic construct
  source 1..311: mol_type = other DNA
caaattgttctcacccagtctccagcaactttgtctgcatctccaggggagaaggctaccatgacctgcagtgccagctcaggtgctaactacatgcactggtaccagcagaagccaggcacctcccccaaaagatggatttatgacacatccaaactggcttctggagtccctgctcgcttcagtggcagtgggtctgggacctcttattctctcacaatcgagagcatggaggctgaagatgctgccacttattactgccatcagcgaggtagttacacgttcggaggggggaccaagctggaaataaa

SEQ ID NO: 12 - DNA, 310 bp, synthetic construct
  source 1..310: mol_type = other DNA
caaattgttctcacccagtctccagcaactttgtctgcatctccaggggagaaggctacatgacctgtagtgccagctcaggtgtcaactacatgcactggtaccagcagaagccaggcacctcccccaaaagatggatttatgacacagacaaaacggcttctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 13 - protein, 120 aa, synthetic construct
QVQLQQPGAEVVKPGASVRLSCKTSGYTFTSNWMHWVKQRPGQGLEWIGEIDPSDSYTNYNQKFKGKAKLTVDKSSSTAYMEVSSLTSEDSAVYYCARGSNPYYYAMDYWGQGTSVTVSS

SEQ ID NO: 14 - protein, 120 aa, synthetic construct
QVQLQQPGAEVVKPGASVRLSCETSGYTFTSNWMHWVKQRPDQGLEWIGEIDPSDSYTNYNQKFKGKAELTVDKSSSTAYMEVSDLTSEDSAVYYCARGSNPYYYAMDYWGQGTSVTVSS

SEQ ID NO: 15 - protein, 120 aa, synthetic construct
QVQLQQPGAEVVKPGASVRLSCKTSGYTFTSNWMHWVKQRPGQGLEWIGEIDPSDSYTNYNQKFKGKAELTVDKSSSTAYMEVSDLTSEDSAVYYCARGSNPYYYAMDYWGQGTSVTVSS

SEQ ID NO: 16 - protein, 120 aa, synthetic construct
QVQLEQPGAEVKKPGASVKVSCKASGYTFTSNWMHWVKQRPGQGLEWIGEIDPSDSYTNYNQKFKGKAKLTVDKSSSTAYMEVSDLTAEDSAVYYCARGSNPYYYAMDYWGQGTSVTVSS

SEQ ID NO: 17 - protein, 120 aa, synthetic construct
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 18 - protein, 120 aa, synthetic construct
QVQLEQPGAEVKKPGASVKVSCKASGYTFTSNWMHWVRQAPGKGLEWIGEIDPSDSYTNYNQKFDGKAKLTVDKSSSTAYMEVSDLTSEDSATYYCARGSNPYYYAMDYWGQGTSVTVSS

SEQ ID NO: 19 - protein, 120 aa, synthetic construct
QVQLEQPGAEVVKPGASVKVSCKTSGYTFTSNWMHWVKQRPGQGLEWIGEIDPSDSYTNYNQKFDGKAKLTVDKSSSTAYMEVSDLTSEDSATYYCARGSNPYYYAMDYWGQGTSVTVSS

SEQ ID NO: 20 - protein, 120 aa, synthetic construct
QVQLQQPGAEVKKPGASVRLSCETSGYTFTSNWMHWVKQRPDQGLEWIGEIDPSDSYTNYNQKFDGKAELTVDKSSSTAYMEVSDLTSEDSAVYYCARGSNPYYYAMDYWGQGTSVTVSS

SEQ ID NO: 21 - protein, 120 aa, synthetic construct
QVQLEQPGAEVKKPGASVKVSCKASGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 22 - protein, 29 aa, synthetic construct
QVQLXQPGAEVXKPGASVXXSCXXSGYTF

SEQ ID NO: 23 - protein, 14 aa, synthetic construct
WVXQXPXXGLEWIG

SEQ ID NO: 24 - protein, 38 aa, synthetic construct
YNQKFXGKAXLTVDKSSSTAYMEVSXLTXEDSAXYYCA

SEQ ID NO: 25 - protein, 104 aa, synthetic construct
QIVLTQSPAIMSASPGEKVTMTCSASSGVNYMHWYQQKPGTSPKRWIYDTSKLASGVPARFSGSGSGTSYSLTISSMEAEDAATYYCHQRGSYTFGGGTKLEIK

SEQ ID NO: 26 - protein, 104 aa, synthetic construct
QIALTQEPAIMSASPGEKVTMTCSASSGVNYMHWYQQKPGTSPKRWIYDTSKLDSGVPARFSGSGSGTSYSLTISSMEAEDAATYYCHQRGSYTFGGGTKLEIK

SEQ ID NO: 27 - protein, 104 aa, synthetic construct
DIVLTQSPATLSASPGEKVTMTCSASSGVNYMHWYQQKPGTSPKRWIYDTSKLDSGVPARFSGSGSGTSYSLTISSMEAEDAATYYCHQRGSYTFGGGTKLEIK

SEQ ID NO: 28 - protein, 104 aa, synthetic construct
QIVLTQSPATLSASPGEKATMTCSASSGANYMHWYQQKPGTSPKRWIYDTSKLASGVPARFSGSGSGTSYSLTIESMEAEDAATYYCHQRGSYTFGGGTKLEIK

SEQ ID NO: 29 - protein, 104 aa, synthetic construct
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIK

SEQ ID NO: 30 - protein, 104 aa, synthetic construct
QIALTQEPAIMSASPGEKATMTCSASSGVNYMHWYQQKPGTSPKRWIYDTSKLDSGVPARFSGSGSGTSYSLTIESMEAEDAATYYCHQRGSYTFGGGTKLEIK

SEQ ID NO: 31 - protein, 104 aa, synthetic construct
QIVLTQSPATLSASPGEKATMTCSASSGANYMHWYQQKPGTSPKRWIYDTDKTASGVPARFSGSGSGTSYSLTIESMEAEDAATYYCHQRGSYTFGGGTKLEIK

SEQ ID NO: 32 - protein, 23 aa, synthetic construct
XIXLTQXPAXXSASPGEKXTMTC

SEQ ID NO: 33 - skipped

SEQ ID NO: 34 - protein, 120 aa, synthetic construct; identical to SEQ ID NO: 13

SEQ ID NO: 35 - protein, 104 aa, synthetic construct; identical to SEQ ID NO: 25

SEQ ID NO: 36 - protein, 9 aa, synthetic construct
VRLSCKTSG

SEQ ID NO: 37 - protein, 9 aa, synthetic construct
WVKQRPGQG

SEQ ID NO: 38 - protein, 9 aa, synthetic construct
YNQKFKGKA

SEQ ID NO: 39 - protein, 9 aa, synthetic construct
FKGKAKLTV

SEQ ID NO: 40 - protein, 9 aa, synthetic construct
YMEVSSLTS

SEQ ID NO: 41 - protein, 9 aa, synthetic construct
VYYCARGSN

SEQ ID NO: 42 - protein, 9 aa, synthetic construct
IVLTQSPAI

SEQ ID NO: 43 - protein, 9 aa, synthetic construct
VLTQSPAIM

SEQ ID NO: 44 - protein, 9 aa, synthetic construct
VTMTCSASS

SEQ ID NO: 45 - protein, 9 aa, synthetic construct
VNYMHWYQQ

SEQ ID NO: 46 - protein, 9 aa, synthetic construct
WIYDTSKLA

SEQ ID NO: 47 - protein, 9 aa, synthetic construct
IYDTSKLAS

SEQ ID NO: 48 - protein, 13 aa, synthetic construct
CGTAAGTGGATCC

SEQ ID NO: 49 - protein, 12 aa, synthetic construct
GCTAGCTCCAGC

SEQ ID NO: 50 - protein, 450 aa, synthetic construct
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 450 aa, synthetic construct; identical to SEQ ID NO: 50

SEQ ID NO: 52 - protein, 211 aa, synthetic construct
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 211 aa, synthetic construct; identical to SEQ ID NO: 52

SEQ ID NO: 54 - protein, 450 aa, synthetic construct; identical to SEQ ID NO: 50

SEQ ID NO: 55 - protein, 211 aa, synthetic construct; identical to SEQ ID NO: 52

(19) United States
(12) Patent Application Publication
(10) Pub. No.: US 2026/0250384 A1
(43) Pub. Date: Aug. 27, 2026

(54) METHODS OF TREATING PMN PATIENTS

(71) Applicant: Climb Bio Operating Inc., Wellesley, MA (US)

(72) Inventors: William Bonificio, Wellesley, MA (US); Stephen Thomas, Wellesley, MA (US)

(21) Appl. No.: 19/355,277

(22) Filed: Oct. 10, 2025

Related U.S. Application Data
(60) Provisional application No. 63/706,220, filed on Oct. 11, 2024.

(51) Int. Cl. C07K 16/28; A61K 39/00; A61P 25/02

(52) U.S. Cl. CPC C07K 16/2803; A61P 25/02; A61K 2039/505; A61K 2039/54; A61K 2039/545; C07K 2317/565

(57) ABSTRACT

The invention provides novel methods for treatment of patients with membranous nephropathy by administration of compositions comprising a therapeutic agent for the treatment of membranous nephropathy. According to the invention, the therapeutic agent for the treatment of membranous nephropathy may be an anti-CD19 antibody.

### I. FIELD OF THE INVENTION

The invention provides novel methods for treatment of patients with membranous nephropathy (MN) by administration of compositions comprising a therapeutic agent for the treatment of membranous nephropathy.

### II. SEQUENCE LISTING

The present application is being filed along with a Sequence Listing XML in electronic format. The Sequence Listing XML is provided as a file named CLIMB-005-01US-Seq-Listing.xml, created on Oct. 9, 2025, which is 55,750 bytes in size. The Sequence Listing XML file and the information in the electronic format of the Sequence Listing XML file is incorporated herein by reference in its entirety.

### III. BACKGROUND

Membranous nephropathy (MN) is an immune-mediated glomerular disease and is the leading cause of non-diabetic nephrotic syndrome (NS) in Caucasian adults. The most prominent clinical feature of MN is NS and its associated manifestations, including various degrees of edema, fatigue, hypoalbuminemia, and hyperlipidemia. Approximately 60% of patients with MN present with nephrotic-range proteinuria (≥3.5 g/day) and the remaining 40% have sub-nephrotic proteinuria, but two-thirds of these latter patients later become nephrotic usually within the first year.

### IV. SUMMARY OF THE INVENTION

The invention provides a method for treatment of patients with MN. In certain preferred embodiments, the invention provides methods for treatment of patients with primary membranous nephropathy (PMN). The method of the invention provides for administration of compositions comprising anti-CD19 antibodies for the treatment of MN and/or PMN. B-cell depletion therapy with the anti-CD20 mAb, rituximab, has shown clinical benefit in patients with MN by reducing proteinuria, presumably by limiting the maturation of B-cells into auto-reactive, antibody-secreting plasmablasts and plasma cells (PCs). However, it was beneficially recognized that CD19 is expressed at an earlier stage in B-cell development, appearing on early pro B-cells, and also persists throughout B-cell maturation on the surface of plasmablasts and some PCs. Accordingly, the methods of the invention provide the administration of anti-CD19 antibodies for elimination of CD19+ plasmablasts.

Thus, the methods of the invention are directed to a method for treatment of MN and/or PMN by the administration of a composition comprising a therapeutically effective amount of an anti-CD19 antibody. The impact of the administration of anti-CD19 antibody for treatment of MN and/or PMN was not previously explored. The methods of the invention beneficially provide that MN and/or PMN patients expressing anti-phospholipase A2 receptor (PLA2R) antibody have a higher response rate for the administration of anti-CD19 antibodies. This correlation of expression of anti-PLA2R antibody on the efficacy of a treatment with anti-CD19 antibodies was surprising and unexpected. Accordingly, in certain aspects, the methods of the invention provide a method for treatment of MN and/or PMN patients by administration of a composition comprising a therapeutically effective amount of an anti-CD19 antibody. In certain embodiments, the anti-CD19 antibody could be any anti-CD19 antibody that binds to the CD19 on the surface of B-cells.

In certain aspects, the invention provides a method of treating a subject having membranous nephropathy (MN), the method comprising: providing a composition comprising a therapeutically effective amount an anti-CD19 antibody to the subject having an anti-phospholipase A2 receptor (PLA2R) antibody, wherein the anti-CD19 antibody comprises: a heavy chain variable region at least 95% identical to SEQ ID NO: 17 and/or a light chain variable region at least 95% identical to SEQ ID NO: 29. In certain embodiments, the anti-CD19 antibody comprises a heavy chain variable region comprising SEQ ID NO: 17 and/or a light chain variable region comprising SEQ ID NO: 29. In certain embodiments, the anti-CD19 antibody comprises a heavy chain variable region comprising SEQ ID NO: 17 and a light chain variable region comprising SEQ ID NO: 29. In certain preferred embodiments, the anti-CD19 antibody is budoprutug.

In certain embodiments, the patient is suffering from primary membranous nephropathy (PMN).

In certain embodiments, the composition comprising the anti-CD19 antibody is administered parenterally. In certain preferred embodiments, the composition is administered via an intravenous (IV) infusion. In certain embodiments, the composition is administered subcutaneously.

In certain embodiments, the composition comprises about 100 mg budoprutug. In certain embodiments, the composition comprises about 200 mg budoprutug. In certain embodiments, the composition comprises about 600 mg budoprutug. In certain embodiments, the composition comprises about 1000 mg budoprutug.

In certain embodiments, the composition comprises 100 mg budoprutug. In certain embodiments, the composition comprises 200 mg budoprutug. In certain embodiments, the composition comprises 600 mg budoprutug. In certain embodiments, the composition comprises 1000 mg budoprutug.

In certain embodiments, the composition is administered once every two weeks. In certain embodiments, the composition is administered at days 1, 15, 169, and 183 after the start of the treatment. In certain embodiments, the composition is administered in treatment cycles, wherein each treatment cycle comprises administering the composition comprising budoprutug once every two weeks, with a 24-week interval between each treatment cycle. In certain embodiments, the composition is administered in treatment cycles, each treatment cycle comprising administration of the composition at days 1 and 15, and wherein there is a 24-week interval between each treatment cycle.

In certain embodiments, the administration of composition results in remission of proteinuria in the patients.

In certain aspects, the invention provides a method for treating a subject having membranous nephropathy (MN), wherein the method comprises testing the subject for the presence of anti-PLA2R antibody in serum. If the subject tests positive for anti-PLA2R antibody, administering a composition comprising a therapeutically effective amount of an anti-CD19 antibody. In certain embodiments, the patient is suffering from primary membranous nephropathy (PMN). In certain preferred embodiments, the anti-CD19 antibody is budoprutug. In certain embodiments, the composition comprising the anti-CD19 antibody is administered parenterally. In certain preferred embodiments, the composition is administered via an intravenous (IV) infusion. In certain embodiments, the composition comprises about 100 mg budoprutug. In certain embodiments, the composition comprises about 200 mg budoprutug. In certain embodiments, the composition is administered once every two weeks. In certain embodiments, the composition is administered at days 1, 15, 169, and 183 after the start of the treatment. In certain embodiments, the administration of composition results in remission of proteinuria in the patients.

In certain aspects, the invention method of determining whether a subject having membranous nephropathy (MN) will respond to treatment with an anti-CD19 antibody, the method comprising: conducting an assay on a sample from the subject that MN in order to determine the presence of anti-PLA2R antibody in the sample; providing results of the assay to the physician such that physician prescribes or provides the subject with a composition comprising a therapeutically effective amount of an anti-CD19 antibody. In certain embodiments, the anti-CD19 antibody comprises a heavy chain variable region at least 95% identical to SEQ ID NO: 17 and/or a light chain variable region at least 95% identical to SEQ ID NO: 29. In certain embodiments, the patient is suffering from primary membranous nephropathy (PMN). In certain preferred embodiments, the anti-CD19 antibody is budoprutug. In certain embodiments, the composition comprising the anti-CD19 antibody is administered parenterally. In certain preferred embodiments, the composition is administered via an intravenous (IV) infusion. In certain embodiments, the composition comprises about 100 mg budoprutug. In certain embodiments, the composition comprises about 200 mg budoprutug. In certain embodiments, the composition is administered once every two weeks. In certain embodiments, the composition is administered at days 1, 15, 169, and 183 after the start of the treatment. In certain embodiments, the administration of composition results in remission of proteinuria in the patients.

### V. BRIEF DESCRIPTION OF THE FIGURES

FIG. 1 provides B-cell count of a patient after administration of budoprutug.

FIG. 2 provides an overview of the participants in the trial.

FIG. 3 provides individual peripheral CD20+ B-cell count versus time following first budoprutug administration for participants with MN.

FIG. 4 provides individual proteinuria (UPCR) versus time following first budoprutug administration for participants with MN.

FIG. 5 provides Sensitivity Analysis: Individual Proteinuria (UPCR) Versus Time Following First Budoprutug Administration for Participants with MN.

FIG. 6 provides collated data for the CD20+ cell count, anti-PLA2R antibodies, and UPCR at various time points after the administration of budoprutug.

FIG. 7, FIG. 8, and FIG. 9 show the IgA, IgG, and IgM versus time following first budoprutug administration for participants with MN for the FAS, respectively.

FIG. 10 provides average anti-PLA2R antibodies over time after the administration of budoprutug.

FIG. 11 shows plasma budoprutug concentration versus time for the PK population.

FIG. 12 and FIG. 13 provide an overview of the clinical trials for Phase 1b clinical trials.

FIG. 14 and FIG. 15 provide an overview of the Phase 2 trial.

### VI. DETAILED DESCRIPTION

The invention provides a method for treatment of patients with MN. In certain preferred embodiments, the invention provides methods for treatment of patients with primary membranous nephropathy (PMN). The method of the invention provides for administration of compositions comprising anti-CD19 antibodies for the treatment of MN and/or PMN. B-cell depletion therapy with the anti-CD20 mAb, rituximab, has shown clinical benefit in patients with MN by reducing proteinuria, presumably by limiting the maturation of B-cells into auto-reactive, antibody-secreting plasmablasts and plasma cells (PCs). However, it was beneficially recognized that CD19 is expressed at an earlier stage in B-cell development, appearing on early pro B-cells, and also persists throughout B-cell maturation on the surface of plasmablasts and some PCs. Accordingly, the methods of the invention provide the administration of anti-CD19 antibodies for elimination of CD19+ plasmablasts.

### Anti-CD19 Antibodies of the Invention:

In certain aspects, the invention provides anti-CD19 antibodies which feature an amino acid sequence defining a modified immunoglobulin heavy chain framework region comprising amino acid residues 1-30 of SEQ ID NO:22, wherein one or more of the amino acid residues at positions X5, X12, X19, X20, X23, and X24 are as follows: X5 is Q or E, X12 is V or K, X19 is R or K, X20 is L or V, X23 is K, E or D, or X24 is T or A. In certain embodiments, at least one of the amino acid residues at positions X5, X12, X19, X20, X23, or X24 is not the same amino acid residue as the amino acid at the corresponding position in the unmodified immunoglobulin heavy chain framework region as set forth in amino acid residues 1-30 of SEQ ID NO:13. In one embodiment, X23 is E or D.

In certain embodiments, the anti-CD19 antibodies feature an amino acid sequence defining a modified immunoglobulin heavy chain framework region comprising amino acid residues 1-14 of SEQ ID NO:23, wherein one or more of the amino acid residues at positions X3, X5, X7, and X8, are as follows: X3 is K or R, X5 is R, T, or A, X7 is G, D, or E, or X8 is Q or K. According to this aspect of the invention, at least one of the amino acid residues at positions X3, X5, X7, or X8 is not the same as the amino acid at the corresponding position in the unmodified immunoglobulin heavy chain framework region as set forth in amino acid residues 36-49 of SEQ ID NO:13. In one embodiment, X7 is E or D.

In certain embodiments, the anti-CD19 antibodies feature an amino acid sequence defining a modified immunoglobulin heavy chain framework region comprising amino acid residues 1-39 of SEQ ID NO:24, wherein one or more of the amino acid residues at positions X6, X10, X26, X29, and X34 are as follows: X6 is K, D, or E, X10 is K, E, or D, X26 is S, D, or E, X29 is S or A, or X34 is V or T. According to this aspect of the invention, at least one of the amino acid residues at positions X6, X10, X26, X29, or X34 is not the same as the amino acid at the corresponding position in the unmodified immunoglobulin heavy chain framework region as set forth in amino acid residues 60-98 of SEQ ID NO: 13. In one embodiment, X10 is E or D.

In certain embodiments, the anti-CD19 antibodies feature an amino acid sequence defining a modified immunoglobulin light chain framework region comprising amino acid residues 1-23 of SEQ ID NO:32, wherein one or more of the amino acid residues at positions X1, X3, X7, X10, X11, and X19 are as follows: X1 is Q or D, X3 is V or A, X7 is S or E, X10 is I or T, X11 is M or L, or X19 is V or A. According to this aspect of the invention, at least one of the amino acid residues at positions X1, X3, X7, X10, X11, or X19 is not the same as the amino acid at the corresponding position in the unmodified immunoglobulin light chain framework region as set forth in amino acid residues 1-23 of SEQ ID NO:25. In one embodiment, X3 is A and X7 is E. In another embodiment, X1 is D, X10 is I, and X11 is L.

In certain embodiments, the invention features an amino acid sequence defining a modified immunoglobulin light chain complementarity determining region comprising amino acid residues 24-33 of SEQ ID NO:28.

In certain embodiments, the invention features an amino acid sequence defining a modified immunoglobulin light chain framework region comprising amino acid residues 56-87 of SEQ ID NO: 28.

According to another aspect, the invention features an antibody variable region comprising an amino acid sequence selected from the group consisting of SEQ ID NO: 14, SEQ ID NO: 15, SEQ ID NO: 16, SEQ ID NO: 17, SEQ ID NO: 20, SEQ ID NO: 21, SEQ ID NO: 26, SEQ ID NO: 27, SEQ ID NO: 28, SEQ ID NO: 29, SEQ ID NO: 30, or SEQ ID NO: 31, wherein the antibody variable region specifically binds to CD19.

In certain embodiments, the invention features a polypeptide at least 90% or at least 95% identical to a B4 antibody heavy chain variable region, the polypeptide comprising an amino acid substitution at one or more residues corresponding to Val12, Leu20, Lys23, Thr24, Lys38, Gly42, Gln43, Lys65, Lys69, Ser85, Ser88, or Val93. In one embodiment, the polypeptide comprises one or more of substitutions Gln5Glu, Val12Lys, Arg19Lys, Leu20Val, Lys23Glu, Lys23Asp, Thr24Ala, Lys38Arg, Arg40Thr, Gly42Asp, Gly42Glu, Gln43Lys, Lys65Asp, Lys65Glu, Lys69Glu, Lys69Asp, Ser85Asp, Ser85Glu, Ser88Ala, or Val93Thr.

According to another aspect, the invention features a polypeptide at least 90% or at least 95% identical to a B4 antibody light chain variable region, the polypeptide comprising an amino acid substitution at one or more residues corresponding to Val3, Ser7, Ile10, Met11, Val19, Val29, Ser51, Leu53, Ala54, or Ser75. In one embodiment, the polypeptide comprises one or more of substitutions Gln1Asp, Val3Ala, Ser7Glu, Ile10Thr, Met11Leu, Val19Ala, Val29Ala, Ser51Asp, Leu53Thr, Ala54Asp, or Ser75Glu.

In certain aspects, the invention provides a process for preparation of anti-CD19 antibody, wherein the anti-CD19 antibody comprises variable domain comprising a light chain variable region that is at least 90% identical to SEQ ID NO: 25 and has an amino acid substitution at one or more residues corresponding to Ile10, Met11, Val19, Ser51, and Leu53; and the process comprises production of the anti-CD19 antibody in Chinese Hamster Ovary (CHO) cells.

In certain aspects, the invention provides a process for preparation of an anti-CD19 antibody, wherein: the anti-CD19 antibody comprises SEQ ID NO: 13 with an amino acid substitution at one or more residues corresponding to Gln5, Arg19, Leu20, Arg40, Gln43, Lys65, Ser85, Ser88, and Val93, and SEQ ID NO: 25 with an amino acid substitution at one or more residues corresponding to Ile10, Met11, Val19, Ser51, and Leu53; and the process comprises production of the anti-CD19 antibody in Chinese Hamster Ovary (CHO) cells.

In certain embodiments, the anti-CD19 antibody variable domain comprises a heavy chain variable region of SEQ ID NO: 17 and a light chain variable region of SEQ ID NO: 29. In certain embodiments, the anti-CD19 antibody is provided in U.S. Pat. No. 8,691,952, which is incorporated by reference in its entirety. In certain embodiments, the anti-CD19 antibody variable domain comprises a heavy chain variable region of SEQ ID NO: 17 and a light chain variable region of SEQ ID NO: 29 are provided in U.S. Pat. No. 8,691,952.

In certain embodiments, the heavy chain variable region of the anti-CD19 antibody has, compared to SEQ ID NO:13, one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, the heavy chain variable region of the anti-CD19 antibody is SEQ ID NO: 13 comprising an amino acid substitution at one or more residues corresponding to Gln5, Arg19, Leu20, Arg40, Gln43, Lys65, Ser85, Ser88, and Val93.

In certain embodiments, the heavy chain variable region of the anti-CD19 antibody has one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, wherein the heavy chain variable region of the anti-CD19 antibody has one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, wherein the heavy chain variable region of the anti-CD19 antibody is the amino acid sequence of SEQ ID NO: 17.

In certain embodiments, the anti-CD19 antibody variable domain of the anti-CD19 antibody further comprises a light chain variable region that is at least 90% identical to SEQ ID NO: 25 and has an amino acid substitution at one or more residues corresponding to Ile10, Met11, Val19, Ser51, and Leu53.

In certain embodiments, wherein the light chain variable region the anti-CD19 antibody has one or more amino acid substitutions selected from the group consisting of Ile10Thr, Met11Leu, Val19Ala, Ser51Asp, and Leu53Thr.

In certain embodiments, the light chain variable region is at least 95% identical to SEQ ID NO: 25.

In certain embodiments, the light chain variable region has one or more amino acid substitutions selected from the group consisting of Ile10Thr, Met11Leu, Val19Ala, Ser51Asp, and Leu53Thr.

In certain embodiments, the light chain variable region is SEQ ID NO: 25 comprising an amino acid substitution at one or more residues corresponding to Ile10, Met11, Val19, Ser51, and Leu53.

In certain embodiments, the light chain variable region has one or more amino acid substitutions selected from the group consisting of Ile10Thr, Met11Leu, Val19Ala, Ser51Asp, and Leu53Thr.

In certain embodiments, the light chain variable region is the amino acid sequence of SEQ ID NO: 29.

In certain embodiments, the heavy chain variable region comprises one or more of substitutions Gln5Glu, Arg19Lys, Leu20Val, Arg40Thr, Gln43Lys, Lys65Asp, Ser85Asp, Ser88Ala, and Val93 Thr.

In certain embodiments, wherein the light chain variable region comprises one or more of substitutions Ile10Thr, Met11Leu, Val19Ala, Ser51Asp, and Leu53Thr.

In certain embodiments, the anti-CD19 antibody is VB119. In certain embodiments, VB119 is an anti-CD19, IgG1 monoclonal antibody. VB119 demonstrates antibody-dependent cell mediated toxicity (ADCC) with minimal to no complement dependent cytotoxicity (CDC).

In certain embodiments, the anti-CD19 antibody comprises one or more of the below listed subunits in the anti-CD19 antibody:

Subunit 1 (SEQ ID NO: 50):
QVQLEQPGAEVVKPGASVKVSCKTSGYTFTSNWMHWVKQTPGKGLEWIGEIDP
SDSYTNYNQKFDGKAKLTVDKSSSTAYMEVSDLTAEDSATYYCARGSNPYYYAMDYW
GQGTSVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Subunit 2 (SEQ ID NO: 51):
QVQLEQPGAEVVKPGASVKVSCKTSGYTFTSNWMHWVKQTPGKGLEWIGEIDP
SDSYTNYNQKFDGKAKLTVDKSSSTAYMEVSDLTAEDSATYYCARGSNPYYYAMDYW
GQGTSVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Subunit 3 (SEQ ID NO: 52):
QIVLTQSPATLSASPGEKATMTCSASSGVNYMHWYQQKPGTSPKRWIYDTDKTAS
GVPARFSGSGSGTSYSLTISSMEAEDAATYYCHQRGSYTFGGGTKLEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
Subunit 4 (SEQ ID NO: 53):
QIVLTQSPATLSASPGEKATMTCSASSGVNYMHWYQQKPGTSPKRWIYDTDKTAS
GVPARFSGSGSGTSYSLTISSMEAEDAATYYCHQRGSYTFGGGTKLEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC

In certain embodiments, the heavy chain sequence of the anti-CD19 antibody is provided below (SEQ ID NO: 54):

QVQLEQPGAEVVKPGASVKVSCKTSGYTFTSNWMHWVKQTPGKGLEWIGEIDPSDSYT
NYNQKFDGKAKLTVDKSSSTAYMEVSDLTAEDSATYYCARGSNPYYYAMDYWGQGTS
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK

In certain embodiments, the light chain sequence of the anti-CD19 antibody is provided below (SEQ ID NO: 55):

QIVLTQSPATLSASPGEKATMTCSASSGVNYMHWYQQKPGTSPKRWIYDTDKTASGVPA
RFSGSGSGTSYSLTISSMEAEDAATYYCHQRGSYTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC

In certain embodiments, the anti-CD19 antibody is budoprutug. In certain embodiments, budoprutug comprises a heavy chain and a light chain. In certain embodiments, budoprutug comprises heavy chain and light chain provided in SEQ ID NO: 54 and SEQ ID NO: 55 respectively.

In certain embodiments, the heavy chain of anti-CD19 antibody is at least 90% identical to SEQ ID NO: 54. In certain embodiments, the heavy chain of anti-CD19 antibody is at least 95% identical to SEQ ID NO: 54. In certain embodiments, the heavy chain of anti-CD19 antibody is at least 98% identical to SEQ ID NO: 54. In certain embodiments, the heavy chain of anti-CD19 antibody is identical to SEQ ID NO: 54.

In certain embodiments, the light chain of anti-CD19 antibody is at least 90% identical to SEQ ID NO: 55. In certain embodiments, the light chain of anti-CD19 antibody is at least 95% identical to SEQ ID NO: 55. In certain embodiments, the light chain of anti-CD19 antibody is at least 98% identical to SEQ ID NO: 55. In certain embodiments, the light chain of anti-CD19 antibody is identical to SEQ ID NO: 55.

### Fc Portion

In certain embodiments, the antibody variable domains of the antibodies prepared using the methods of the invention are optionally fused to an Fc portion. As used herein, the Fc portion encompasses domains derived from the heavy chain constant region of an immunoglobulin, preferably a human immunoglobulin, including a fragment, analog, variant, mutant or derivative of the constant region. The constant region of an immunoglobulin heavy chain is defined as a naturally-occurring or synthetically produced polypeptide homologous to at least a portion of the C-terminal region of the heavy chain, including the CH1, hinge, CH2, CH3, and, for some heavy chain classes, CH4 domains. The “hinge” region joins the CH1 domain to the CH2-CH3 region of an Fc portion. The constant region of the heavy chains of all mammalian immunoglobulins exhibit extensive amino acid sequence similarity.

In the present invention, the Fc portion typically includes at least a CH2 domain. For example, the Fc portion can include the entire immunoglobulin heavy chain constant region (CH1-hinge-CH2-CH3). Alternatively, the Fc portion can include all or a portion of the hinge region, the CH2 domain and the CH3 domain.

The constant region of an immunoglobulin is responsible for many important antibody effector functions, including those mediated by Fc receptor (FcR) binding and by complement binding. There are five major classes of the heavy chain constant region, classified as IgA, IgG, IgD, IgE, and IgM, each with characteristic effector functions designated by isotype.

IgG, for example, is separated into four γ isotypes: γ1, γ2, γ3, and γ4, also known as IgG1, IgG2, IgG3, and IgG4, respectively. IgG molecules can interact with multiple classes of cellular receptors including three classes of Fcγ receptors (FcγR) specific for the IgG class of antibody, namely FcγRI, FcγRII, and FcγRIII. The sequences important for the binding of IgG to the FcγR receptors have been reported to be in the CH2 and CH3 domains.

It is also often useful to alter the serum half-life of the antibody. The serum half-life of an antibody, as of an immunoglobulin fusion protein, is influenced by the ability of that antibody to bind to an Fc receptor (FcR) (Gillies et al., Cancer Research (1999) 59:2159-66). The CH2 and CH3 domains of IgG2 and IgG4 have undetectable or reduced binding affinity to Fc receptors compared to those of IgG1. Accordingly, the serum half-life of the featured antibody can be increased by using the CH2 and/or CH3 domain from IgG2 or IgG4 isotypes. Alternatively, the antibody can include a CH2 and/or CH3 domain from IgG1 or IgG3 with modification in one or more amino acids in these domains to reduce the binding affinity for Fc receptors (see, e.g., U.S. patent application Ser. No. 09/256,156, published as U.S. patent application publication 2003-0105294, which is incorporated by reference in its entirety).

In certain embodiments, an Fc portion fused to an antibody variable region of the invention can contain CH2 and/or CH3 domains and a hinge region that are derived from different antibody isotypes. For example, the Fc portion can contain CH2 and/or CH3 domains of IgG2 or IgG4 and a hinge region of IgG1. Assembly of such hybrid Fc portions has been described in U.S. patent application publication 2003-0044423, which is incorporated by reference in its entirety.

When fused to an antibody variable region of the invention, the Fc portion may contain one or more amino acid modifications that generally extend the serum half-life of an Fc fusion protein. Such amino acid modifications include mutations substantially decreasing or eliminating Fc receptor binding or complement fixing activity. For example, one type of such mutation removes the glycosylation site of the Fc portion of an immunoglobulin heavy chain. In IgG1, the glycosylation site is Asn297 (see, for example, U.S. patent application Ser. No. 10/310,719, published as U.S. patent application publication 2003-0166163).

### Membranous Nephropathy:

The majority of patients present nephrotic syndrome. The complications associated with nephrotic syndrome include edema, hyperlipidemia, thrombosis, and infections. Remission of nephrotic patients with MN increases the chances of survival. Kidney Int. 2004 September; 66 (3): 1199-205.

Rituximab, a monoclonal antibody against the cell surface antigen CD20 of B cells, was reported to safely reduce proteinuria and ameliorate nephrotic syndrome (NS) in eight patients with primary membranous nephropathy (MN) and severe NS unresponsive to prolonged angiotensin-converting-enzyme (ACE) inhibitor therapy. J Am Soc Nephrol. 2015 October; 26 (10): 2545-58. N Engl J Med. 2019 Jul. 4; 381 (1): 36-46.

In approximately 70% of adult patients with primary MN autoantibodies against an antigen normally expressed on the podocyte cell, the M-type phospholipase A2 receptor (PLA2R) circulates and binds to conformational epitopes present on PLA2R, producing in situ deposits characteristic of the disease. Circulating anti-PLA22R autoantibodies are almost exclusively specific to patients with primary MN offered clinicians and scientists a powerful tool to differentiate primary from secondary forms and monitor disease activity and response to therapy.

Combination therapies for MN have been previously explored. For example, rituximab and Cytoxan were utilized for treatment of MN with prednisone. Cortazar, Frank B. et al. KI Reports 2018; 3 (2): 94-402. Am J Kidney Dis. 2021 December; 78 (6): 793-803.

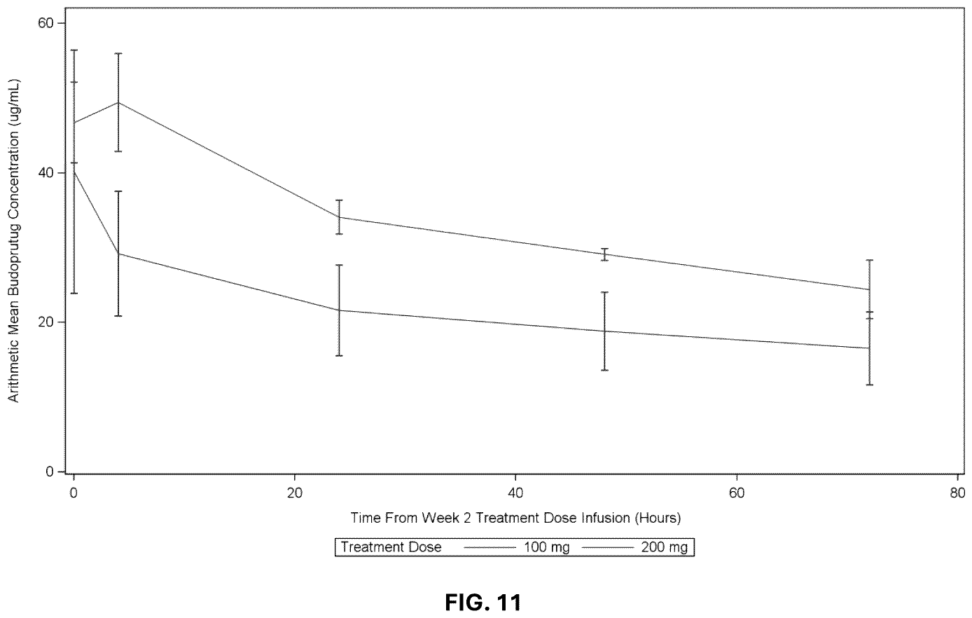

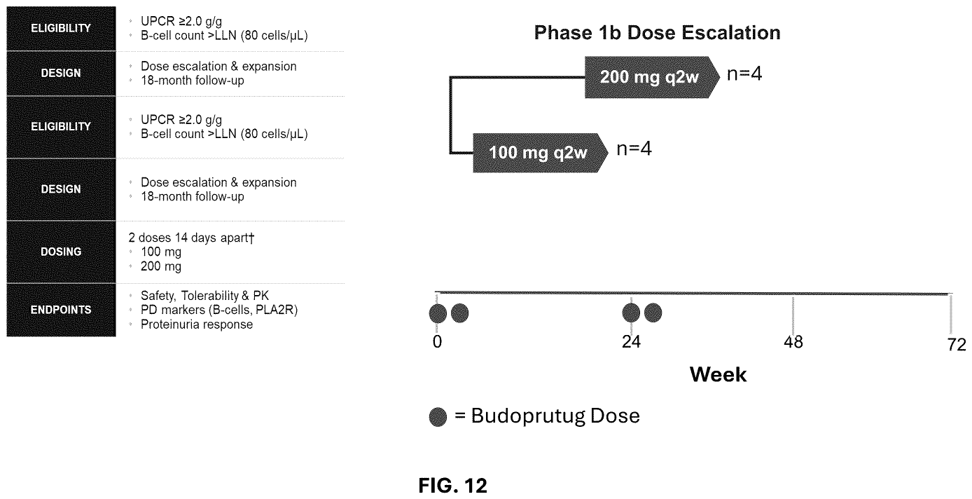

FIG. 12 and FIG. 13 provide an overview of the clinical trials for Phase 1b clinical trials.

### METHODS OF THE INVENTION

Thus, the methods of the invention are directed to a method for treatment of MN and/or PMN by the administration of a composition comprising a therapeutically effective amount of an anti-CD19 antibody. The impact of the administration of anti-CD19 antibody for treatment of MN and/or PMN was not previously explored. The methods of the invention beneficially provide that MN and/or PMN patients expressing anti-phospholipase A2 receptor (PLA2R) antibody have a higher response rate for the administration of anti-CD19 antibodies. This correlation of expression of anti-PLA2R antibody on the efficacy of a treatment with anti-CD19 antibodies was surprising and unexpected. Accordingly, in certain aspects, the methods of the invention provide a method for treatment of MN and/or PMN patients by administration of a composition comprising a therapeutically effective amount of an anti-CD19 antibody. In certain embodiments, the anti-CD19 antibody could be any anti-CD19 antibody that binds to the CD19 on the surface of B-cells.

In certain aspects, the invention provides a method of treating a subject having membranous nephropathy (MN), the method comprising: providing a composition comprising a therapeutically effective amount an anti-CD19 antibody to the subject having an anti-phospholipase A2 receptor (PLA2R) antibody, wherein the anti-CD19 antibody comprises: a heavy chain variable region at least 95% identical to SEQ ID NO: 17 and/or a light chain variable region at least 95% identical to SEQ ID NO: 29. In certain embodiments, the patient is suffering from primary membranous nephropathy (PMN). In certain preferred embodiments, the anti-CD19 antibody is budoprutug.

In certain embodiments, the composition comprising the anti-CD19 antibody is administered parenterally. In certain preferred embodiments, the composition is administered via an intravenous (IV) infusion. In certain embodiments, the composition is administered subcutaneously.

In certain embodiments, the composition comprises about 100 mg budoprutug. In certain embodiments, the composition comprises about 200 mg budoprutug. In certain embodiments, the composition comprises about 600 mg budoprutug. In certain embodiments, the composition comprises about 1000 mg budoprutug.

In certain embodiments, the composition comprises 100 mg budoprutug. In certain embodiments, the composition comprises 200 mg budoprutug. In certain embodiments, the composition comprises 600 mg budoprutug. In certain embodiments, the composition comprises 1000 mg budoprutug.

In certain embodiments, the composition is administered once every two weeks. In certain embodiments, the composition is administered at days 1, 15, 169, and 183 after the start of the treatment. In certain embodiments, the composition is administered in treatment cycles, wherein each treatment cycle comprises administering the composition comprising budoprutug once every two weeks, with a 24-week interval between each treatment cycle. In certain embodiments, the composition is administered in treatment cycles, each treatment cycle comprising administration of the composition at days 1 and 15, and wherein there is a 24-week interval between each treatment cycle.

In certain aspects, the invention provides a method for treating a subject having membranous nephropathy (MN), wherein the method comprises testing the subject for the presence of anti-PLA2R antibody in serum. If the subject tests positive for anti-PLA2R antibody, administering a composition comprising a therapeutically effective amount of an anti-CD19 antibody. In certain embodiments, the patient is suffering from primary membranous nephropathy (PMN). In certain preferred embodiments, the anti-CD19 antibody is budoprutug. In certain embodiments, the composition comprising the anti-CD19 antibody is administered parenterally. In certain preferred embodiments, the composition is administered via an intravenous (IV) infusion. In certain embodiments, the composition comprises about 100 mg budoprutug. In certain embodiments, the composition comprises about 200 mg budoprutug. In certain embodiments, the composition is administered once every two weeks. In certain embodiments, the composition is administered at days 1, 15, 169, and 183 after the start of the treatment. In certain embodiments, the administration of composition results in remission of proteinuria in the patients.

In certain aspects, the invention method of determining whether a subject having membranous nephropathy (MN) will respond to treatment with an anti-CD19 antibody, the method comprising: conducting an assay on a sample from the subject that MN in order to determine the presence of anti-PLA2R antibody in the sample; providing results of the assay to the physician such that physician prescribes or provides the subject with a composition comprising a therapeutically effective amount of an anti-CD19 antibody. In certain embodiments, the anti-CD19 antibody comprises a heavy chain variable region at least 95% identical to SEQ ID NO: 17 and/or a light chain variable region at least 95% identical to SEQ ID NO: 29. In certain embodiments, the patient is suffering from primary membranous nephropathy (PMN). In certain preferred embodiments, the anti-CD19 antibody is budoprutug. In certain embodiments, the composition comprising the anti-CD19 antibody is administered parenterally. In certain preferred embodiments, the composition is administered via an intravenous (IV) infusion. In certain embodiments, the composition comprises about 100 mg budoprutug. In certain embodiments, the composition comprises about 200 mg budoprutug. In certain embodiments, the composition is administered once every two weeks. In certain embodiments, the composition is administered at days 1, 15, 169, and 183 after the start of the treatment. In certain embodiments, the administration of composition results in remission of proteinuria in the patients.

### Budoprutug:

Budoprutug (VB119) is a humanized, low-fucosylated, anti-Cluster of Differentiation (CD) 19, immunoglobulin (Ig) G1, monoclonal antibody (mAb) with potent antibody-dependent cellular toxicity as determined by cell-based assays. The target antigen, CD19, is expressed on many Ig-producing B-cells, making it an attractive target for Ig-mediated diseases. Budoprutug has been shown to inhibit proliferation of a wide range of B-cell lines at lower effective concentrations of rituximab. Budoprutug was also found to deplete B-cells in severe combined immunodeficient (SCID) mice injected with human primary peripheral blood mononuclear cells (PBMCs). Unlike rituximab, budoprutug has been shown to have minimal complement-dependent cytotoxicity (CDC) in preclinical models. Complement-dependent cytotoxicity is a likely cause of the infusion-related reactions (IRRs) of antibody therapies.

In a Phase 1 clinical study in patients with B-cell malignancies, a total of 28 patients were treated with budoprutug doses ranging from 0.02 mg to 1000 mg weekly for up to 4 weeks. By Week 4, ≥70% B-cell depletion was observed in 25 out of 28 patients. Of the 20 patients with evaluable data who received at least 250 mg of budoprutug, 17 out of 20 (85%) patients experienced complete B-cell depletion, defined as >90% reduction in CD20+ B-cells compared to baseline during at least 1 timepoint during the study period, with some responses lasting beyond 6 months from the last dose.

No human anti-human antibodies (HAHA) were detected, and budoprutug was tolerated at all doses studied. The majority of adverse events (AEs; such as IRRs and Grade 3 or higher neutropenia) and the 3 study withdrawals all occurred in patients in the highest dose cohort (1000 mg). Importantly, because the 1000 mg cohort was expanded, two-thirds of the total study population was treated at this dose level, which provides additional safety experience at the highest dose, but also limits the ability to compare frequencies of uncommon AEs. Serum immunoglobulins (IgA, IgM, and IgG) were assessed at baseline, during the study, and at the End of Therapy and follow-up visits. Twenty-seven (96.4%) patients had a value that was outside the normal range, but none of these abnormalities were considered clinically significant.

Phase Ib Trial of Budoprutug in Adult Subjects with PMN:

Membranous nephropathy (MN) is an immune-mediated glomerular disease and is the leading cause of non-diabetic nephrotic syndrome (NS) in Caucasian adults. The most prominent clinical feature of MN is NS and its associated manifestations, including various degrees of edema, fatigue, hypoalbuminemia, and hyperlipidemia. Approximately 60% of patients with MN present with nephrotic-range proteinuria (≥3.5 g/day) and the remaining 40% have sub-nephrotic proteinuria, but two-thirds of these latter patients later become nephrotic usually within the first year.

The most consequential long-term manifestation of MN is progressive loss of renal function, which occurs in 60% of untreated patients with about 35% eventually developing end-stage renal disease (ESRD) within 10 years. The long-term outcomes correlate with the degree of proteinuria, and thus, proteinuria is an established surrogate marker for disease control and progression. Patients presenting with nephrotic-range proteinuria (i.e., ≥3.5 g/day) have worse outcomes than those presenting with sub-nephrotic proteinuria, but even those presenting with sub-nephrotic proteinuria remain at risk for the disease consequences over time.

Treatment for MN is aimed specifically at reducing proteinuria. More recently, a robust correlation of proteinuria and serum anti-phospholipase A2 receptor (PLA2R) antibody levels has been observed, with anti-PLA2R antibody levels being a leading indicator of proteinuria. Serum anti-PLA2R antibody levels are increasingly gaining acceptance as a predictive surrogate marker of disease and response to treatment. It is common for anti-PLA2R antibody levels to decrease weeks to months following the initiation of treatment, and for proteinuria to decrease in the weeks to months following anti-PLA2R antibody reduction.

### Rationale:

B-cell depletion therapy with the anti-CD20 mAb, rituximab, has shown clinical benefit in patients with MN by reducing proteinuria, presumably by limiting the maturation of B-cells into auto-reactive, antibody-secreting plasmablasts and plasma cells (PCs). CD19 is expressed at an earlier stage in B-cell development, appearing on early pro B-cells, and also persists throughout B-cell maturation on the surface of plasmablasts and some PCs. The clinical implications of this differential expression of CD19 are not yet fully understood. Preclinical data suggest that the elimination of CD 19+ plasmablasts and PCs could provide a greater clinical effect in B-cell-driven autoimmune diseases than targeting CD20+ B-cells.

Patients with autoimmune disease, including multiple sclerosis, have been shown to have greater numbers of CD19+ B-cells in serum when compared to healthy controls. Plasmablasts and PCs have been suggested to be a primary effector B-cell population involved in the secretion of immunoglobulins leading to autoantibody mediated inflammation, and CD19+ B-cell niches persist following treatment with rituximab. In MN specifically, circulating PCs are elevated in diseased patients compared to controls, and plasmablasts from patients have been shown to produce pathogenic anti-PLA2R autoantibodies. Therefore, there is a scientific rationale for depleting CD19+ B-cells in patients with autoimmune diseases, including glomerular diseases such as primary MN (pMN).

Though rituximab has provided encouraging clinical data in managing proteinuria in MN, non-responder rates remain high. Experts believe that the duration of time until patient relapse tends to shorten following subsequent rounds of rituximab therapy. Rituximab's ability to deplete B-cells is restricted to cells expressing CD20 antigen, which is typically absent on plasmablasts and PCs. Furthermore, rituximab remains an unapproved, off-label option for patients with MN, which often limits access and lacks clear prescribing guidelines. Thus, targeting CD19 with a cell-depleting antibody has the potential to provide safe and effective elimination of B-cells, including pathologically relevant plasmablasts and PCs.

### Risk Benefit:

Anti-CD19 mAb therapy is expected to deplete B-lymphocytes. Budoprutug has been shown to reduce B-cell populations in a Phase 1 clinical trial (CRUKD/12/003) in patients with advanced CD19-positive indolent B-cell malignancies. The duration of this intended drug effect is anticipated to be temporary, lasting weeks to months, and may increase the risk of serious and opportunistic infections. To reduce the infection risk, participants with acute infections were not enrolled, and all participants were pre-screened and excluded if they tested positive for certain opportunistic pathogens, such as tuberculosis and hepatitis. Participants reporting signs or symptoms of infection following administration of budoprutug therapy were promptly evaluated and treated appropriately.

Budoprutug may also induce neutropenia, which was observed in some patients with B-cell lymphomas and chronic lymphocytic leukemia after administration of the drug. Hematological oncology patients are more susceptible to neutropenia, and experience with the B-cell depleting agent rituximab shows the risk of neutropenia is greater in patients with hematological malignancies compared to autoimmune populations. Nevertheless, the protocol excluded participants with abnormal neutrophil counts at baseline and regularly monitored neutrophil levels.

The primary acute risk of budoprutug administration was the possibility of an IRR, including cytokine release syndrome (CRS), which can occur from the administration of any therapeutic antibody. This is a well-recognized aspect of clinical practice with chimeric, humanized, or fully human antibodies, regardless of their mode of action. There is no supportive data from other clinical trials to suggest that anti-CD19 antibodies are more prone to this effect than other types of antibodies; however, in order to minimize the risk of reactions, participants who had experienced prior IRRs to rituximab were excluded from the study. Participants who received budoprutug received premedication prior to each infusion. This premedication was in line with standard practice for administration of other mAbs, including the recently approved anti-CD19 antibody Uplizna™. Participants were carefully monitored for signs of CRS or IRRs during and after each infusion. If participants developed any signs, such as urticaria, wheezing, hypotension, tachycardia, or dyspnea, the infusion was stopped immediately, and additional treatment administered.

### Primary Objective

The primary objective of this study was to define the safety and tolerability of ascending doses of budoprutug in participants with PMN.

### Secondary Objectives:

The secondary objectives of this study included the following:
To investigate a range of dose levels for budoprutug in participants with MN;
To characterize the pharmacokinetic (PK) profile of budoprutug in participants with MN;
To evaluate the effects of budoprutug on B-cell depletion (pharmacodynamic [PD] response);
To evaluate the effects of budoprutug on serum anti-PLA2R antibodies (immunologic response);
To evaluate the effects of budoprutug on proteinuria (clinical response);
To evaluate the effects of budoprutug on albuminuria and albuminemia;
To evaluate the effects of budoprutug on NS;
To evaluate the effects of budoprutug on serum IgG, IgM, and IgA levels; and
To evaluate the development of anti-drug antibodies (ADAs) to budoprutug.

### Exploratory Objectives:

The exploratory objectives of this study included the following:
To evaluate participant reported outcomes/Quality of Life (QOL) measures, including edema; and
To evaluate the PK and PK/PD (dose relationship) profile overall and as they relate to proteinuria levels.

### Overall Study Design and Plan:

This study was a Phase 1b, open-label, sequential-cohort, dose escalation study to evaluate the safety, tolerability, PK, and PD of budoprutug in participants with PMN. Participants received 2 or 4 doses of budoprutug, 14 days apart (1 dose on Day 1 and 1 dose on Day 15 [protocol Versions 1.0-6.0] and 1 dose on Day 168 and 1 dose on Day 182 [protocol Versions 1.0-5.0]) at the specified dose level, administered via intravenous (IV) infusion.

For all participants enrolled, retrospective data was collected from medical records for up to 2 years prior to Screening to document the participant's pre-treatment disease history. These data included any available renal biopsy, estimated glomerular filtration rate (eGFR), anti-PLA2R antibody titers, proteinuria, and B-cell/lymphocyte levels, as well as MN treatments received. To minimize bias, these data was only collected following signing of informed consent and confirmation that the participant met all study eligibility criteria.

Participants were screened for study eligibility at Visit 1 (Visit 1a or Visit 1b, as applicable, for participants who required tapering of immunosuppressive medications, including calcineurin inhibitors [CNIs] and/or glucocorticoids). After Screening, participants were reassessed at Visit 2/Day 1 and all participants who met the eligibility criteria were enrolled into the study.

The study used a starting dose based on the results of the completed first-in-human Phase 1 study. Dose escalation based on safety and B-cell depletion was conducted using a flexible sample size (3 to 6 participants) for each cohort. The first dose cohort was 100 mg administered IV on Day 1 and on Day 15 for 2 doses (200 mg cumulative dose). The planned dose cohort that followed was 200 mg administered IV on Day 1 and on Day 15 for 2 doses (400 mg cumulative dose). The dose escalation cohort would have been enrolled sequentially. The overall goal was to demonstrate PD effects with 2 bi-weekly doses. Escalation in dose between the cohorts would not exceed more than double the previous cohort's cumulative dose. Following the first dosing cohort, the plan was flexible based on decisions of the Data Review Committee (DRC), focusing primarily on safety and PD effects of B-cell depletion. Following initiation of the first study cohort, the subsequent cohort would have been opened for enrolment following review of the data and determinations of the DRC.

The study collected data on safety and tolerability, PK, B-cell depletion, serum anti-PLA2R antibody levels, and proteinuria.

Four sites in the United States opened for this study.

### Study Duration

The total study duration for each individual participant was up to 88 weeks, including Screening (up to 18 weeks), Treatment Period (initially 2 bi-weekly and 2 possible bi-weekly maintenance doses starting at 6 months [Week 24]), and a Follow-up Visit. The total study duration following the first dose was up to 72 weeks.

### Data Review:

Each cohort in the dose escalation phase were followed for at least 5 weeks after dose initiation in at least 3 participants prior to escalation decisions. The following data was reviewed by the DRC to determine the addition of participants within a cohort and escalation to the next dose cohort:
Relevant information on participants' demographics, characteristics, medical history, physical examinations, and concomitant medications;
Available PD data (e.g., B-cell subsets [including CD20+ B-cell counts]);
List of all AEs, including severity, time of onset related to study drug administration, duration, relatedness, and causality, including serious adverse events (SAEs), adverse events of special interest (AESIs), and all potential dose-limiting toxicities (DLTs);
Any clinically significant, out of range safety clinical laboratory test results;
Electrocardiogram (ECG) and vital sign results, including clinically significant abnormal values;
Available PK data; and
Any available anti-PLA2R antibody and proteinuria data from the current cohort or prior cohort.

Within the planned dose range, a dose lower than the next planned dose may have been tested, depending on review of available safety, efficacy, tolerability, PK, and PD data. If the highest planned dose level was found to be safe and tolerable, also considering the PK and PD data, additional higher doses may have been added through a protocol amendment.

### Dose Escalation:

Participants with PMN were assigned to the open cohort, per the Table below. The dose escalation cohort was enrolled sequentially. Three participants were enrolled into the first cohort initially, with an option of enrolling up to 6 participants total in a cohort based on the DRC recommendation of additional participants for safety assessments or more complete assessment of B-cell depletion. As the participant follow-up data were generated over time, anti-PLA2R antibody levels and proteinuria were also factored into the evaluation of cohort size. During and following each cohort, the DRC reviewed the available safety, efficacy, tolerability, PK, and PD data to determine the addition of participants within a cohort and escalation from Cohort 1 to Cohort 2. Escalation from Cohort 1 to Cohort 2 only took place after DRC review. The next dosing cohort began at Visit 2/Day 1.

### Doses of Budoprutug

| Dose | Dose | |
|---|---|---|
| Level | (mg) | Frequency (Cumulative Dose) |
| Level 1 | 100 | Every 14 days for 2 doses (200 mg cumulative dose) |
| Level 2 | 200 | Every 14 days for 2 doses (400 mg cumulative dose) |

For safety, if none of the initial 3 participants experienced a DLT during the initial treatment period, then enrolment into the next dose cohort could begin. If 1 of the first 3 participants experienced a DLT, then dosing was stopped. If study enrolment was stopped due to a DLT, the DRC reviewed all available safety data to determine if the study could resume at the current dose, resume at a lower dose, proceed to a study amendment to address safety concerns, or terminate. For PD effects, if the data from 3 participants did not demonstrate consistent effects, then up to 3 more participants could be enrolled at that dose level to better understand the PD effects prior to escalation to the next higher dose cohort. The PD response target was >90% reduction in peripheral CD20+ B-cell counts and/or B-cell counts≤25 cells/μL, and optimally this response occurred within 2 weeks of dose initiation.

Participants who withdrew from the study for reasons other than a DLT prior to completing the 1-month follow-up after the second bi-weekly dose could be replaced to ensure an adequate safety and PD assessment for each cohort. Replacement participants were enrolled to the same dose cohort as the participant being replaced.

### Definition of Dose-Limiting Toxicities:

Dose-limiting toxicities due to a study drug were generally defined as a toxicity that prevents further administration of the study drug at that dose level. Based on preclinical and human-dosing in B-cell malignancies with budoprutug, fatigue, nausea, vomiting, neutropenia, and IRRs were the most common AEs. The reported DLTs in that experience included IRR and hypoxemia.

Study recruitment would stopped and a DRC meeting would occur if any participant experienced any of the following toxicities which were considered possibly, probably, or definitely related to study drug:
1. Any SAE;
2. Severely decreased neutrophil count (i.e., absolute neutrophil count<500/mm);
3. Moderately decreased neutrophil count of prolonged duration (i.e., absolute neutrophil count 500 to 1000/mm³ for ≥7 days);
4. Severe infection, defined as requiring IV antibiotics, antifungal, and/or antiviral treatment and/or hospital admission for >24 hours and/or is considered life-threatening;
5. Serum IgG levels<250 mg/dL for ≥4 months;
6. IRR of any severity that requires stopping of study drug infusion or requires treatment with additional medications or interventions to address the signs and symptoms;
7. Hypoxia (hypoxemia, hypoxaemia, hypoxic) with or without IRR;
8. Nausea of duration≥7 days;
9. Vomiting of duration≥3 days;
10. Fatigue of duration≥7 days; or

Any AE or toxicity, at the discretion of the Investigator, that is thought to warrant withholding the study drug.

Study recruitment would be stopped and a DRC meeting would occur if any participant experienced any of the following events:
1. Acute kidney injury (AKI) stage 1 or higher, defined by the Kidney Disease Improving Global Outcomes (KDIGO) 2012 Clinical Practice Guideline, and does not demonstrate immediate reversibility as suggested by a serum creatinine level that does not improve below the level of the AKI definition or worsens with repeat confirmation of the serum creatinine within 7 days upon notification;
2. Thromboembolic events (e.g., deep-venous thrombosis, renal vein thrombosis, pulmonary embolism);
3. Worsening of proteinuria by >50% from baseline; or
4. Worsening of measures of the NS which, in the opinion of the Investigator, may preclude ongoing participation. For example, new onset anasarca or severe hypoalbuminemia.

If study enrolment was stopped due to DLTs or disease-specific AEs/conditions as noted above, the DRC reviewed all available safety data to determine if the study could resume at the current dose, resume at a lower dose, proceed to a study amendment to address safety concerns, or terminate. Note that any amendments to address stopping rules was considered substantial amendments of the protocol. If the study was stopped after reaching a stopping rule, health authorities were to be notified, as required, and a substantial amendment was to be filed to request study restart.

Throughout the study, participants were seen at protocol-defined timepoints to assess safety, B-cell depletion, serum anti-PLA2R antibodies, proteinuria, albuminemia, and eGFR. This enabled detection of change from baseline, the time to effect, and duration of effect. In addition, the patterns of each parameter were evaluated; it was anticipated that B-cell depletion would be followed by anti-PLA2R antibody reduction, and then proteinuria reduction. Proteinuria was measured as urine protein-to-creatinine ratio (UPCR) from either a 24 hour collection or spot urine collections. Other measures of proteinuria were also measured, including urinary albumin excretion and total daily protein excretion.

All participants had a follow-up End of Study (EOS) Visit at either Week 72 or when the study was terminated. This duration of follow up allowed the collection of additional safety data and data on the persistence of B-cell depletion, anti-PLA2R antibody reduction, proteinuria reduction, and overall MN disease activity over time.

### Inclusion Criteria:

Participants who met all the following criteria were eligible to participate in the study:
1. Was ≥18 years of age at the time of informed consent;
2. Had a kidney biopsy-proven diagnosis of pMN within the past 10 years;
   Note: It was preferable that participants enrolled have kidney biopsy tissue samples that were positive for anti-PLA2R antibody staining. Participants with kidney biopsy-proven diagnosis of pMN>10 years and ≤20 years that met all other eligibility criteria could be enrolled after discussion with the Medical Monitor.
3. Had a documented laboratory history of nephrotic range proteinuria (defined as either ≥3.5 g total protein per 24-hour urine collection or >3.5 g/g UPCR by spot collection) AND had proteinuria with a UPCR≥2.0 g/g based on 2 consecutive spot urine (first morning void) sample collections obtained within 14 days of each other during the Screening Period. Both samples must have qualified;
4. Was on maximum allowable or maximum tolerated anti-proteinuric therapy with a renin angiotensin system inhibitor (RASi; i.e., angiotensin converting enzyme inhibitor or angiotensin II receptor blocker) whose dose must have been stable for ≥6 months prior to Screening (as well as anticipated to be stable during the study). A stable dose of RASi was defined as doses that were within 50% of the planned dose for ≥6 months prior to Screening. Participants with short-term interruptions of RASi therapy (e.g., for volume-depleting illness or medication refills) and those who were intolerant to or contraindicated for RASi therapy may have been enrolled upon discussion with the Medical Monitor;
5. Had systolic blood pressure (BP)<160 mmHg and diastolic BP<100 mmHg after 5 minutes of rest at Screening;
6. Was considered by the Investigator to be an appropriate candidate for immunosuppressive therapy according to international guidelines (KDIGO 2012/2020);
7. Was willing and able to provide written informed consent prior to Screening;
8. Female participants of non-childbearing potential must have been either surgically sterile (hysterectomy, bilateral tubal ligation, salpingectomy, and/or bilateral oophorectomy at least 26 weeks before the Screening Visit) or postmenopausal, defined as spontaneous amenorrhea for at least 2 years, with follicle-stimulating hormone in the postmenopausal range at Screening, based on the central laboratory's ranges;
9. Female participants of childbearing potential (i.e., ovulating, premenopausal, or not surgically sterile) and all male participants were to use a medically accepted, highly effective contraceptive regimen during their participation in the study and for 125 days after the last administration of study drug. Highly effective contraceptive methods were defined as those with 99% or greater efficacy;
   a. Acceptable methods of contraception for male participants enrolled in the study included the following:
      i. Condoms with spermicide; or
      ii. Surgical sterilization of participant at least 26 weeks before Screening (vasectomy);
   b. Acceptable methods of contraception for female participants of childbearing potential enrolled in the study included the following:
      i. Intrauterine device for at least 12 weeks before Screening;
      ii. Hormonal contraception (oral, implant, injection, ring, or patch) for at least 12 weeks before Screening. Hormonal contraceptives were only acceptable if the contraceptive prevented ovulation; or
      iii. True abstinence;
         True abstinence: When this was in line with the preferred and usual lifestyle of the participant. Periodic abstinence (such as calendar, ovulation, symptothermal, post-ovulation methods) and withdrawal were not acceptable methods of contraception.
10. Male participants agreed to abstain from sperm donation through 125 days after administration of the last dose of study drug; and
   Had recovered from a previous SARS-COV-2 infection or received a SARS-COV-2 vaccination at least 2 weeks prior to Screening.

### Exclusion Criteria:

Participants who met any of the following criteria were excluded from participating in the study:
1. Had received treatment with rituximab or other B-cell depleting agents within 6 months prior to Screening;
2. Had a CD19+ B-cell count<80 cells/μL at Screening or <40 cells/μL if B-cell depleting treatment was received 6 months to 2 years prior to Screening;
3. Had an eGFR<45 mL/min/1.73 m2 at Screening utilizing the Chronic Kidney Disease Epidemiology Collaboration formula confirmed by the central laboratory;
4. Had a known AKI event in the last 3 months. If a participant's recent decline in eGFR was thought to be attributable to MN, they could have been enrolled upon discussion with the Medical Monitor;
5. Had achieved a >50% reduction in proteinuria over the previous 6 months;
6. Had an absolute neutrophil count<1.5×109/L;
7. Had a white blood cell count<3.0×109/L;
8. Had an aspartate aminotransferase (AST) and/or alanine aminotransferase (ALT)>2× upper limit of normal (ULN);
   Note: Participants with AST and/or ALT>2×ULN at Screening could have repeat laboratory samples drawn approximately 1 week later. Participants were considered eligible after repeat laboratory samples if repeat value was ≤ULN and the participant had no known liver disease.
9. Had a total bilirubin>1.5×ULN, unless due to Gilbert's syndrome;
   Note: Participants with total bilirubin>1.5×ULN at Screening could have repeat laboratory samples drawn approximately 1 week later. Participants were considered eligible after repeat laboratory samples if repeat value was ≤ULN and the participant had no known liver disease.
10. Had a history of serious or significant infusion reaction associated with rituximab or other B-cell depleting therapy;
11. Had secondary causes of MN (e.g., malignancy, hepatitis B or C, human immunodeficiency virus [HIV], systemic lupus erythematosus [SLE], or other autoimmune diseases [e.g., thyroiditis, sarcoidosis], or drug-induced);
12. Had a diagnosis or history of SLE (including non-renal disease);
13. Had type 1 or type 2 diabetes mellitus;
14. Had concomitant or overlapping glomerulopathies on renal biopsy or known by the participant's clinical history (e.g., diabetic nephropathy, IgA nephropathy, or primary or genetic variant focal segmental glomerulosclerosis [FSGS]). Those with secondary or adaptive FSGS could be enrolled upon discussion with the Medical Monitor;
15. Had an acute, chronic, or latent infection, including tuberculosis, hepatitis, HIV, or chronic urinary tract infections;
16. Had a live vaccine within 28 days prior to the first scheduled day of budoprutug administration or planned to obtain a live vaccine during the study;
17. Had used glucocorticoids or mycophenolate mofetil within 14 days prior to Screening;
   Note: The Screening Period could be extended to allow for the tapering of glucocorticoids or mycophenolate mofetil over approximately 90 days, provided that the participant was off the medication for at least 14 days prior to the first dose of budoprutug.
18. Had used alkylating agents (e.g., cyclophosphamide) within 2 months prior to Screening;
19. Had used azathioprine within 14 days prior to Screening;
   Note: The Screening Period could be extended to allow for the tapering of azathioprine over approximately 90 days, provided that the participant was off the medication for at least 14 days prior to the first dose of budoprutug.
20. Had used or was using CNIs at the time of enrolment:
   a. Participants that had used CNIs, including cyclosporine and tacrolimus, within 14 days of the first dose of budoprutug;
   b. The Screening Period could be extended to allow for the tapering of CNIs over approximately 90 days, provided that the participant was off the medication for at least 14 days prior to the first dose of budoprutug.
21. Had undergone stem cell, bone marrow, or solid organ transplant;
22. Had a history of malignancy within the past 5 years, or was likely to require chemotherapeutic or surgical treatment during the study, with the exception of successfully treated non-melanoma skin cancer, cervical carcinoma in situ, or ductal carcinoma in situ;
23. Was pregnant or lactating female;
24. Had received any investigational drug within 30 days prior to the first scheduled day of budoprutug administration, or at least 5 times the elimination half-life of the investigational drug, whichever was longer;
25. Had a history of drug or alcohol abuse within 5 years prior to Screening;
26. Had a planned surgery during the 6 weeks following the first scheduled day of budoprutug administration;
27. Had a known sensitivity to any components (i.e., sodium citrate, citric acid, or L-arginine) in the budoprutug formulation; or
28. Had any other clinically significant disease, condition, or medical history that, in the opinion of the Investigator, would interfere with participant safety, study evaluations, and/or study procedures.

### Removal Criteria:

Participation of a participant in this clinical study could be discontinued for any of the following reasons:
The participant withdrew consent or requested discontinuation from the study for any reason;
Occurrence of any medical condition or circumstance that exposed the participant to substantial risk and/or did not allow the participant to adhere to the requirements of the protocol;
Any SAE, DLT, clinically significant AE, severe laboratory abnormality, intercurrent illness, or other medical condition which indicated to the Investigator that continued participation was not in the best interest of the participant;
Pregnancy;
The participant failed to comply with protocol requirements or study-related procedures; or
Termination of the study by the Sponsor or the regulatory authority.

If a participant withdrew prematurely from the study due to the above criteria or any other reason, study staff should make every effort to complete the full panel of assessments scheduled for the Early Termination (ET) Visit. The reason for participant withdrawal was documented in the electronic case report form (eCRF).

In the case of participants lost to follow-up, attempts to contact the participant were made and documented in the participant's medical records.

Participants who withdrew from the study for reasons other than a DLT prior to completing the 1-month follow-up after the second bi-weekly dose could be replaced to ensure an adequate safety and PD assessment for each cohort. Replacement participants were enrolled to the same dose cohort as the participant being replaced.

### Treatment:

Participants with pMN were enrolled to the open cohort as described previously (100 mg or 200 mg). The dose escalation cohort was enrolled sequentially.

Three participants were enrolled into the first cohort initially, with an option of enrolling up to 6 participants total in a cohort based on the DRC recommendation of additional participants for safety assessments or more to complete assessment of B-cell depletion. As participant follow-up data was generated over time, anti-PLA2R antibody levels and proteinuria could also be factored into the evaluation of cohort size.

For safety, if none of the initial 3 participants experienced a DLT during the initial treatment period, then enrolment into the next dose cohort could begin. If 1 of the first 3 participants experienced a DLT, then dosing would stop. If study enrolment was stopped due to a DLT, the DRC reviewed all available safety data to determine if the study could resume at the current dose, resume at a lower dose, proceed to a study amendment to address safety concerns, or terminate. For PD effects, if the data from 3 participants did not demonstrate consistent effects, then up to 3 more participants could be enrolled at that dose level to better understand the PD effects prior to escalation to the next higher dose cohort. The PD response target was >90% reduction in peripheral CD20+ B-cell counts and/or B-cell counts≤25 cells/μL, and optimally this response would occur within 2 weeks of dose initiation.

### Rationale for Dosing:

The proposed starting dose of budoprutug was 100 mg administered IV on Day 1 and on Day 15 for 2 doses (200 mg cumulative dose, Cohort 1, Table below). This dose level was informed by safety and PD data from a previous Phase 1 clinical study in patients with B-cell malignancies. The proposed starting dose was anticipated to be well tolerated and result in partial, transient B-cell depletion. Cohort 2 received 200 mg administered IV on Day 1 and on Day 15 for 2 doses (400 mg cumulative dose).

### Cohort Dosing

| | | Dose | Total No. | Cumulative |
|---|---|---|---|---|
| Cohort | Dose Level | Frequency | of Doses | Dose |
| 1 (starting | 100 mg | Every 14 days | 2 | 200 mg |
| dose) | | | | |
| 2 | 200 mg | Every 14 days | 2 | 400 mg |

A Phase 1 clinical study in participants with advanced B-cell malignancies provided first-in-human safety and tolerability data for budoprutug. In this study, participants were administered cumulative doses of budoprutug ranging from 0.87 mg to 4000 mg, and single doses ranging from 0.02 mg to 1000 mg administered once weekly for 4 weeks; the first 3 participants underwent intra-participant dose escalation. Twenty-seven participants in the safety population (n=28) experienced at least 1 treatment-emergent adverse event (TEAE) during the study and 26 participants had a TEAE that was considered related (possibly, probably, or highly probably) to budoprutug. The majority of TEAEs reported were classified as Common Terminology Criteria for Adverse Events (CTCAE) Grade 1 or Grade 2. A total of 11 participants reported 29 TEAEs that were Grade≥3, the majority of which were events of myelosuppression (e.g., low lymphocytes, platelets, neutrophils, and white blood cells) and occurred in participants who were in the 1000 mg dose once weekly cohort. Detailed safety data are available in Section 7.3.1.1 of the Investigator's Brochure.

Six of the 28 participants (21%) had treatment-emergent SAEs reported. Four participants experienced 5 SAEs that were considered related to budoprutug (neutropenic sepsis, two events of IRR [1 of which was classified as a DLT], and 2 events of urinary tract infection in 1 participant, who withdrew from the study). Three of these participants were in 1000 mg once weekly cohorts and 1 participant (neutropenic sepsis) was in the 250 mg once weekly cohort.

Infusion-related reactions were an event of interest during the study, and participants were carefully monitored for signs of CRS or acute infusion reaction during and following each infusion. Pretreatment in most participants consisted of administration of paracetamol, chlorphenamine, and hydrocortisone prior to administration of budoprutug. Five TEAEs of IRR were reported by 4 participants, all in 1000 mg once weekly dose cohorts. All of the IRRs were deemed probably or highly probably related to budoprutug.

Sixteen participants in the study did not experience any treatment-emergent SAEs or Grade≥3 AEs, including all 3 participants in the first 3 dose cohorts. One of these participants (Participant 36/003 of the third dose cohort) received a cumulative dose (225 mg) most similar to the newly proposed starting dose in the MN study. Following single doses of 15, 30, 60, and 120 mg budoprutug, the cumulative area under the concentration-time curve (AUC; 253 μg/mL× day) in this participant was within 10% of the predicted cumulative AUC (277 μg/mL× day) of the proposed starting dose of 100 mg administered IV every 14 days for 2 doses (200 mg cumulative dose) over the 4-week period.

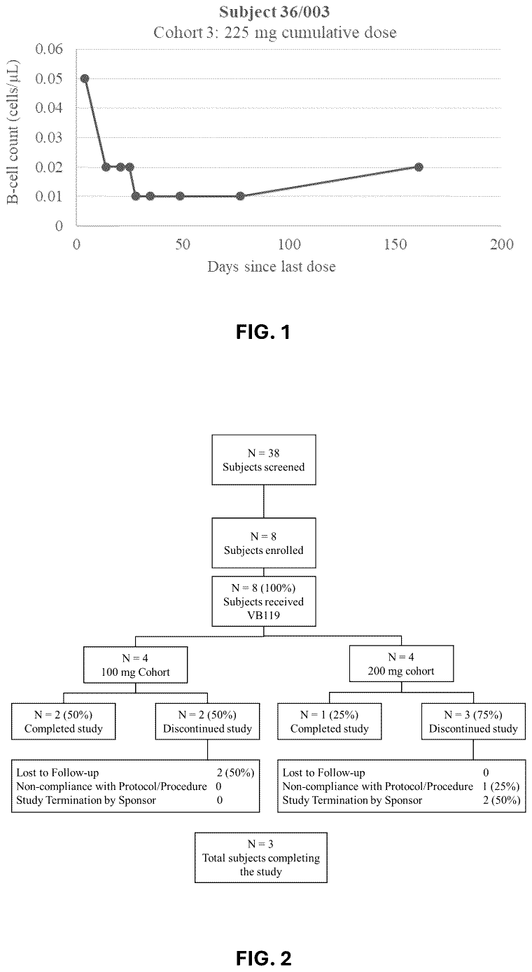

Based on PD data from the Phase 1 study in oncology patients, B-cell depletion occurred following 2 of 4 weekly doses in most participants. Participant 46/002 of the second cohort received a cumulative dose of 15 mg and transient reductions in B-cells were noted; peripheral B-cell counts initially dropped below 10 cells/μL but were above the lower limit of normal (>80 cells/μL) within 4 weeks following the last dose. Participant 36/003 of the third cohort received 4 escalating doses of budoprutug at 15, 30, 60, and 120 mg (225 mg cumulative dose). Prior to Week 4 dosing, and after a cumulative dose of 105 mg administered, B-cell counts were reduced ~60% from the earliest available timepoint (FIG. 1). The last dose of 120 mg resulted in ~80% depletion from the earliest available timepoint. B-cell reduction in this participant persisted through Week 12, although B-cells increased above Week 12 levels by the 6-month follow-up. Based on these data the proposed starting dose of 100 mg administered IV on Day 1 and on Day 15 for 2 doses (200 mg cumulative dose) is anticipated to lead to partial and transient reductions in B-cell counts.

The proposed cumulative dose range of 200 mg to 400 mg in the MN study was below the 1000 mg cumulative dose of the first multi-participant (n=3) cohort (250 mg once weekly) in the previous Phase 1 study in cancer patients. Although the 1000 mg cumulative dose was considered to be tolerated in the oncology study, lower doses could be adequate to achieve clinically meaningful B-cell depletion in participants eligible for the proposed MN study. The treatment goal in participants with autoimmune diseases, including MN, when given other B-cell depleting antibodies (i.e., rituximab, inebilizumab) was typically >90% B-cell depletion from baseline for ≥6 months.

Pharmacokinetic modelling indicates that plasma concentrations of budoprutug would continue to increase during the 2 bi-weekly doses as the drug remains within its distribution phase. Following the final dose of the initial treatment period, budoprutug plasma concentrations were expected to remain detectable for weeks, but fall more rapidly in participants with proteinuria, based on budoprutug PK modelling and data from other antibodies that have been tested in participants with proteinuria, including rituximab, adalimumab, eculizumab, and belimumab. The effect of proteinuria on the PK and PD of budoprutug will be explored in the current study.

Of note, the Phase 1 oncology study evaluated 4 weekly doses. While 4 weekly doses may be necessary for complete and durable B-cell depletion, participant-level data show that, in the majority of participants, B-cell depletion occurs after the first 1 or 2 doses. The drug half-life (approximately 25 days) supports less frequent dosing, and other B-cell depleting therapies (i.e., rituximab and inebilizumab) are administered once every 2 weeks for a total of 2 doses. Accordingly, this study design for budoprutug in MN participants commenced with a low-dose cohort at 2 bi-weekly doses.

In summary, based on available data from the oncology study, 100 mg administered IV on Day 1 and on Day 15 for 2 doses (200 mg cumulative dose) was anticipated to be a well-tolerated starting dose in moderate-to-high risk MN patients and could induce transient B-cell reductions for several weeks. Higher and potentially more frequent doses could be required to induce greater, sustained B-cell depletion and, in turn, disease control (improvement in proteinuria and NS) in moderate-to-high risk patients with MN.

### Formulation:

Budoprutug drug product was provided as a colourless to slightly opalescent liquid containing 10.0 mg/mL budoprutug antibody formulated in 10 mM citrate, and 30 g/L L-arginine-HCl. It had a pH of 6.0 and was presented in a 5 mL glass vial with a 5 mL fill.

### Administration of the Drug:

Study drug was administered by site staff during site visits.

Budoprutug was administered IV on Day 1 and on Day 15 for 2 doses at the 100 mg dose level (200 mg cumulative dose).

Dosing occurred no less than 14 days and no more than 16 days from the previous dose at Visit 4.

Budoprutug was diluted in 0.9% saline at a concentration of no more than 1.7 mg/mL for administration as an IV infusion. For standardization in this study, budoprutug was diluted in 250 mL prefilled saline bags prior to administration. During the first dosing cohort (100 mg bi-weekly) the infusion administered to each participant was initiated at a rate of 40 mg/hour (100 mL/hour). In the absence of infusion toxicity, the infusion rate could be increased by 20 mg/hour (50 mL/hour) increments every 30 minutes. The infusion rate could be the same or higher for the other dosing cohort, based upon DRC review of available safety data. Participants were observed for at least 1 hour following each infusion to monitor for potential infusion reactions. A Study Pharmacy Manual provided more specific information on budoprutug preparation and administration.

Participants were required to receive premedication prior to each infusion of budoprutug to reduce the frequency and severity of infusion reactions. Acceptable types of premedication are shown in the Table below.

Participants with minor symptoms that settled quickly after treatment interruption could restart budoprutug administration with a reduction in the infusion rate. Participants who experienced severe, life-threatening reactions to budoprutug did not receive any further infusions of budoprutug and were withdrawn from study treatment but were followed for required safety follow-up.

TABLE
Premedication Required Prior to Each Budoprutug Infusion

| | | | Administration |
|---|---|---|---|
| | Route of | | Time prior to |
| Type of | Adminis- | Examples (or | Budoprutug |
| Medication | tration | Equivalent) | Infusion |
| Corticosteroid | Intravenous | Methylpednisolone 80 | 30 minutes |
| | | mg | |
| Antihistamine | Oral | Diphenhydramine 50 mg, | 30 to 60 |
| | | Loratadine 10 mg, or | minutes |
| | | Cetirizine 10 mg | |
| Antipyretic | Oral | Acetaminophen/ | 30 to 60 |
| | | paracetamol 1000 mg | minutes |

Study drug was administered only under the supervision of the Investigator by delegated site staff. The Investigator or other appropriate site staff were responsible for maintaining specific participant records of study drug dispensed and administered, including dosage and date and time of study drug administration, in the participant's source documents and the eCRF.

### Storage:

All vials were stored upright and refrigerated (between 2° C. to 8° C. [36° F. to 46° F.]) at the study site. All study drugs were stored in a secure, temperature-controlled refrigerator, with access limited to site staff involved in the study.

### Prior and Concomitant Medications:

All participants who did not require tapering of medications could proceed to enrolment following confirmation of all inclusion and exclusion criteria. Participants received the first dose of budoprutug within 14 days of completion of the second spot urine sample after eligibility was confirmed.

### Excluded Medications:

Participants who had received treatment with the following within the specified timeframe were excluded from the study:
Rituximab or other B-cell depleting agents within 6 months prior to Screening;
Glucocorticoids or mycophenolate mofetil within 14 days prior to Screening; Note: The Screening Period could be extended to allow for the tapering of glucocorticoids or mycophenolate mofetil over approximately 90 days, provided that the participant was off the medication for at least 14 days prior to the first dose of budoprutug.
Alkylating agents within 2 months prior to Screening;
Azathioprine within 14 days prior to Screening;
Note: The Screening Period could be extended to allow for the tapering of azathioprine over approximately 90 days, provided that the participant was off the medication for at least 14 days prior to the first dose of budoprutug.
CNIs, including cyclosporine and tacrolimus, within 14 days of the first dose of budoprutug; or
Note: The Screening Period could be extended to allow for the tapering of CNIs over approximately 90 days, provided that the participant was off the medication for at least 14 days prior to the first dose of budoprutug. For all participants who underwent tapering of their immunosuppressive medication(s), an additional Qualifying Visit occurred once the taper had been completed and within 7 days of completion of the taper.
Live vaccines within 28 days prior to the first dose of budoprutug.

### Rescue Therapy:

During the study, the Investigator could administer or prescribe rescue therapy (e.g., glucocorticoids with additional concomitant immunosuppressive agents) for participants with recurring or persistent nephrotic-range proteinuria, worsening symptomatology, and/or declining kidney function. The Medical Monitor was consulted for advice prior to initiation.

When rescue therapy was required, the Investigator tapered the rescue medications as soon as clinically appropriate to avoid risk of over-immunosuppression and to better assess the effects of budoprutug. Participants who received rescue medication were evaluated for continuation in the study by the Investigator, who could consult with the Medical Monitor and the Sponsor. Participants who discontinued complete all ET procedures as indicated in the schedule of study procedures (refer to the Table below).

TABLE
Schedule of Screening Procedures:

| | Screening Period | | | | | | | | |
|---|---|---|---|---|---|---|---|---|---|
| | | Qualifying | | | | | Follow-Up | EOT | ET |
| | Screening | Visit^{c} | Treatment Period^{a} | | | | Visit | visit | Visit |
| | Visit | | | | | | | | |
| | 1a | 1b | 2^{d} | 3^{d} | 4^{d} | 5^{d,n} | 6 | 7 | |
| | Week | | | | | | | | |
| | −18 to −1 | −5 to −1 | 1 | 2 | 3 | 4 | 7 | 11 | |
| | Day (Visit Window) | | | | | | | | |
| | | | | 8 | 15 | 22 | 43 | 71 | |
| | | | | (+2 | (+2 | (+2 | (+2 | (+7 | |
| | −126 to −1 | −35 to −1 | 1 | days) | days) | days) | days) | days) | UNS |
| Informed | X | | | | | | | | |
| consent | | | | | | | | | |
| Demographics | X | | | | | | | | |
| I/E criteria^{e} | X | X | X | | | | | | |
| Medical/surgical | X | X | X | | | | | | |
| history^{f} | | | | | | | | | |
| Prior and | X | | | | | | | | |
| concomitant | | | | | | | | | |
| medications | | | | | | | | | |
| ADA evaluation | X | X | X | | X | | X | X | X |
| HBcAb, | X | | | | | | | | |
| HBsAb, | | | | | | | | | |
| HBsAg, HCV, | | | | | | | | | |
| HIV, TB testing | | | | | | | | | |
| Height, weight, | X | X | X | X | X | X | X | X | X |
| BMI^{g} | | | | | | | | | |
| Vital signs^{h} | X | X | X | X | X | X | X | X | X |
| Complete | X | | | | | | | X | X |
| physical | | | | | | | | | |
| examination | | | | | | | | | |
| Symptom-based | | X | X | X | X | X | X | | |
| physical | | | | | | | | | |
| examination | | | | | | | | | |
| Clinical | X | X | X | X | X | X | X | X | X |
| chemistry tests | | | | | | | | | |
| Clinical | X | X | X | X | X | X | X | X | X |
| hematology | | | | | | | | | |
| tests | | | | | | | | | |
| Urinalysis | X | X | X | X | X | X | | X | X |
| FSHⁱ | X | | | | | | | | X |
| Pregnancy | X | X | X | X | X | X | X | X | X |
| testing^{j} | | | | | | | | | |
| Study drug | | | X | | X | | | | |
| administration^{k} | | | | | | | | | |
| PK evaluation | | | X | X | X | X | X | X | X |
| Urine testing^{m} | X^{n,o} | X | | | | | X | X | X |
| Anti-PLA2R | X | X | X | | | | X | X | X |
| antibody sample | | | | | | | | | |
| B-cell subset | | | | | | | | X | X |
| evaluation | | | | | | | | | |
| IgA, IgG, and | | | | | | | | X | X |
| IgM | | | | | | | | | |
| assessments | | | | | | | | | |
| 12-lead ECG | | | | | | | | X | X |
| QOL | | | | | | | | X | X |
| assessment | | | | | | | | | |
| AE collection | X | | | | | | | | |
| and reporting | | | | | | | | | |

^{a}During and following each cohort, the DRC reviewed the available safety, efficacy, tolerability, PK, and PD data to determine the addition of participants within a cohort and escalation to the next dose cohort. Escalation to the next higher dose only took place after DRC review. The next dosing cohort began at Visit 2/Day 1.
^{b}All participants who did not require tapering of CNIs and/or glucocorticoids could proceed to enrollment following confirmation of all I/E criteria. For participants who did not require tapering of their current medications, two spot urine (first morning void) collections were completed within 14 days of each other during the Screening Period, butwas initiated within 1 week of the Screening Visit, to confirm eligibility. Participants received the first dose of budoprutug within 14 days of completion of the second spot urine sample after eligibility was confirmed.
^{c}For participants who used restricted medications as outlined in the I/E criteria (i.e., glucocorticoids, mycophenolate, azathioprine, CNIs [Phase 1b]) within 14 days prior to Screening, the Screening Period were extended to allow for their tapering over approximately 90 days, provided that the participant was off the medication(s) for at least 14 days prior to the first dose of budoprutug. Participants in Phase 2a also could taper CNIs during the study (e.g., if complete remission of proteinuria is achieved), though it was not required. For participants required to taper their current medications, a Qualifying Visit occurred within 7 days of completion of the taper, and the first of two spot urine (first morning void) collections occurred within 7 days of completion of the taper. The two spot urine sample collections were completed within 14 days of each other. Participants received the first dose of budoprutug within 14 days of completion of the second spot urine sample after eligibility was confirmed.
^{d}All assessments were completed pre-infusion on days where study drug was administered, with the exception of applicable PK samples.
^{e}Eligibility criteria were reviewed at Screening and the Qualifying Visit (as applicable) and Visit 2/Day 1. Participants were required to meet all criteria prior to the first dose of budoprutug.
^{f}For all participants enrolled, retrospective data were collected from medical records for up to 2 years prior to Screening to document the participants' pre-treatment disease history. These data included any available renal biopsy, eGFR, anti-PLA2R antibody titers, proteinuria, and B-cell/lymphocyte levels, as well as MN treatments received.
^{g}Height was measured at Screening only. Weight was collected at each specified visit. BMI was calculated at Screening.
^{h}Vital signs measured included heart rate, temperature, systolic and diastolic BP, and respirations.
ⁱFSH was only performed in postmenopausal females, defined as spontaneous amenorrhea for at least 2 years, who were not surgically sterile.
^{j}A serum hCG was drawn at Screening. A urine hCG test was performed during the Treatment Period (Visits 2 through 5), Follow-up Visit 6, and at ET.
^{k}Budoprutug was administered IV on Day 1 and on Day 15 for 2 doses at the 100 mg dose level (200 mg cumulative dose) and subsequent bi-weekly dosing schedules at Visit 2 and Visit 4. Dosing occurred no less than 14 days and no more than 16 days from the previous dose at Visit 4. Participants were required to receive premedication prior to each infusion of budoprutug to reduce the frequency and severity of infusion reactions. Participants were observed for at least 1 hour following each infusion to monitor for potential infusion reactions.
^{l}PK samples collected before the infusion were collected within 5 minutes of the start of the infusion. Samples collected at the end of infusion had a collection window of ±3 minutes. Samples collected 4 hours post the end of the infusion had a window of ±30 minutes. Samples collected 24 hours post the end of the infusion had a window of ±1 hour. Samples collected >24 hours post the end of the infusion had a window of ±2 hours. Urine PK samples were collected at the Week 7 visit from the spot urine collection.
^{m}Urine testing included albumin, UPCR, UACR, total creatinine, and urine total protein, based on a spot urine (first morning void) sample collection. Supplies for at-home spot urine sample collection were provided to all participants at the visit prior to when the next collection was due. A spot urine collection was repeated if the Investigator, Medical Monitor, or Sponsor suspects that the sampling was insufficient or inadequate. If a repeat sample was required, the timeline for returning the sample could be extended by 1 week (7 days). If a repeat sample was required during the Screening Period, the sample collection occurred no more than 14 days prior to the first dose of budoprutug.
ⁿSupplies for spot urine sample collection were provided to participants at this visit. Participants were instructed to begin collecting spot urine samples within 5 days of the next scheduled visit and to return all samples to the study site no more than 4 days prior to the next scheduled visit or at the next scheduled visit.
^{o}In order to ensure collections were returned in a timely manner during the Screening and Qualifying Visit, participants were to return the spot urine collections within 5 days of each completion. For participants who did not require tapering of their current medications, two spot urine (first morning void) collections were completed within 14 days of each other during the Screening Period, but initiated within 1 week of the Screening Visit, to confirm eligibility. For participants who required to taper their current medications, the first of two spot urine (first morning void) collections occurred within 7 days after completion of the taper. The two spot urine sample collections was completed within 14 days of each other. Participants received the first dose of budoprutug within 14 days of completion of the second spot urine sample after eligibility was confirmed.
ADA = anti-drug antibody; AE = adverse event; anti-PLA2R = anti-phospholipase A2 receptor; BMI = body mass index; BP = blood pressure; CNI = calcineurin inhibitor; DRC = Data Review Committee; ECG = electrocardiogram; eGFR = estimated glomerular filtration rate; EOS = End of Study; ET = Early Termination; FSH = follicle-simulating hormone; HBcAb = hepatitis B core antibody; HBsAb = hepatitis B surface antibody; HBsAg = hepatitis B surface antigen; hCG = human chorionic gonadotropin; HCV = hepatitis C virus; HIV = human immunodeficiency virus; I/E = inclusion and exclusion; IgA = immunoglobulin A; IgG = immunoglobulin G; IgM = immunoglobulin M; IV = intravenous(ly); MN = membranous nephropathy; PD = pharmacodynamic(s); PK = pharmacokinetic(s); QOL = Quality of Life; TB = tuberculosis; UACR = urine albumin-to-creatinine ratio; UNS = unscheduled; UPCR = urine protein-to-creatinine ratio.

### Documentation of Prior and Concomitant Medication Use:

All prior and concomitant medications were documented in the source documents and applicable eCRFs. Concomitant medications were assessed and recorded at every study visit from the time of informed consent until study participation was complete.

For all participants enrolled, retrospective data was collected from medical records for up to 2 years prior to Screening, to document the participant's pre-treatment disease history. These data included any available renal biopsy, eGFR, anti-PLA2R antibody titers, proteinuria, and B-cell/lymphocyte levels, as well as MN treatments received. To minimize bias, these data were only collected following signing of informed consent and confirmation that the participant met all study eligibility criteria.

### Study Procedures:

All assessments were to be completed pre-infusion on days where study drug was administered, with the exception of applicable PK samples.

### Efficacy Measurements:

There were no primary efficacy endpoints.
The secondary efficacy endpoints included the following:
Budoprutug PK parameters (including area under the maximum observed concentration, time to maximum observed concentration, terminal half-life, apparent clearance, and volume of distribution);
The change from baseline in absolute peripheral CD20+ B-cell count;
The proportion of participants achieving >90% B-cell depletion and/or B-cell count≤25 cells/μL;
The change from baseline in proteinuria (via UPCR) by spot urine collection measurements;
The proportion of participants achieving complete remission of proteinuria (i.e., UPCR≤0.3 g/g);
The proportion of participants achieving a reduction from their baseline proteinuria (i.e., UPCR>0.3 g/g but <3.5 g/g and ≥50% reduction of proteinuria from baseline);
The proportion of participants achieving ≥50% reduction of proteinuria from baseline;
The change from baseline in albuminuria by spot urine collection measurements or albuminemia;
The proportion of participants achieving ≥50% reduction of albuminuria or ≥25% increase in albuminemia from baseline;
The proportion of participants who develop NS on-study and/or proportion of participants whose NS resolves on-study (NS is defined as urinary protein excretion ≥3.5 g/g, hypoalbuminemia, and edema);
The change in serum IgA, IgG, and IgM from baseline over time; and
The incidence of participants who develop ADAs at any time after study drug administration.
   For the subset of participants who are anti-PLA2R antibody positive at baseline, additional secondary efficacy endpoints include the following:
The change from baseline in anti-PLA2R antibody levels;
The proportion of anti-PLA2R antibody-negative participants (defined as <2 RU/mL) over time;
The proportion of participants achieving ≥50% reduction of serum anti-PLA2R antibody levels from baseline over time; and
The proportion of participants achieving >90% reduction of serum anti-PLA2R antibody levels from baseline over time.
   The exploratory endpoints included the following:
The change in participant reported outcomes/QOL measures, including edema (Patient-Reported Outcomes Measurement Information System [PROMIS]);
PK and its relationship to B-cell depletion and clinical response (e.g., anti-PLA2R antibody levels and proteinuria); and
Dose and its relationship to B-cell depletion and clinical response.

### B-Cell Subset Evaluation:

Samples were collected for B-cell subset evaluation at visits specified in the Table above. For B-cell evaluations during Month 1 (Visits 2 through 4), samples were collected weekly. B-cell subset evaluations included CD19+ and CD20+ B-cell counts. All B-cell subset evaluation samples were collected pre-infusion at visits where participants received study drug.

### Immunoglobulin Assessment:

Samples for Ig assessment (including IgA, IgG, and IgM) were collected at visits specified in the Table above. All samples for Ig assessment were collected pre-infusion at visits where participants received study drug.

### Anti-Drug Antibody Evaluation:

Samples for ADA evaluation were collected at visits specified in the Table above. At visits where study drug was administered, samples for ADA evaluation were collected pre-infusion.

### Pharmacokinetic Assessment:

Serum PK samples were collected per the Table below. Samples collected before the infusion were collected within 5 minutes of the start of the infusion. Samples collected at the end of infusion had a collection window of ±3 minutes. Samples collected 4 hours post the end of the infusion had a window of ±30 minutes. Samples collected 24 hours post the end of the infusion had a window of ±1 hour. Samples collected >24 hours post the end of the infusion had a window of ±2 hours.

TABLE
Serum PK Sampling Time Points

| Initial | |
|---|---|
| Treatment | |
| Period | Serum PK Sampling Times in Relation to Infusion |
| Week 1 | Before infusion, EOI, and 4, 24, 48, and 72 hours post-EOI |
| Week 2 | 1 PK sample |
| Week 3 | Before infusion, EOI, and 4, 24, 48, and 72 hours post-EOI |
| Week 4 | 1 PK sample |
| | Day 29 (1 sample) |
| | Day 43 (1 sample |
| | Day 71 (1 sample) |

EOI = end of infusion;
PK = pharmacokinetic(s)

### Urine-Testing:

Urine testing was performed at visits specified in the Table for Scheduling procedures above. Urine testing included albumin, UPCR, urine albumin-to-creatinine ratio (UACR), total creatinine, and urine total protein, based on a spot urine (first morning void) sample collection at each specified measurement timepoint. For participants who did not require tapering of their current medications, two spot urine (first morning void) collections was completed within 14 days of each other during the Screening Period, but must have been initiated within 1 week of the Screening Visit, to confirm eligibility. Participants received the first dose of budoprutug within 14 days of completion of the second spot urine sample after eligibility was confirmed. Baseline was defined as the average of the Screening or Qualifying Visit (if applicable) UPCR values.

For participants who required to taper their current medications, a Qualifying Visit occurred within 7 days of completion of the taper, and the first of two spot urine (first morning void) collections occurred within 7 days of completion of the taper. The two spot urine sample collections was completed within 14 days of each other. Participants received the first dose of budoprutug within 14 days of completion of the second spot urine sample after eligibility was confirmed.

Supplies for at-home spot urine sample collection was provided to all participants at the visit prior to when the next collection was due. A spot urine collection could be repeated if the Investigator, Medical Monitor, or Sponsor suspects that the sampling was insufficient or inadequate. If a repeat sample was required, the timeline for returning the sample could be extended by 1 week (7 days). If a repeat sample was required during the Screening Period, the sample collection occurred no more than 14 days prior to the first dose of budoprutug.

Supplies for spot urine sample collection was provided to participants at visits specified in the Table for Scheduling procedures above. All participants received instructions in order to increase compliance and accurate data collection for the spot urine sample collections. Participants were instructed to begin collecting spot urine samples within 5 days of the next scheduled visit and to return all samples to the study site no more than 4 days prior to the next scheduled visit or at the next scheduled visit. In order to ensure collections were returned in a timely manner during the Screening and Qualifying Visit, participants were to return the spot urine collections within 5 days of each completion.

### Anti-PLA2R Antibody-Testing:

Anti-PLA2R antibody testing was performed at visits specified in the Table for Scheduling procedures above. All samples for anti-PLA2R antibody testing were collected pre-infusion at visits where participants received the study drug.

The EUROIMMUN anti-PLA2R (IgG) test kit was fully CE-marked for this purpose and met the requirements of relevant European product directives.

### Quality of Life Assessment:

Quality of life was assessed at visits specified in the Table for Scheduling procedures above. The PROMIS was used to assess participant QOL. Edema was also measured as part of the QOL assessment using an edema self-assessment tool. All QOL assessments was collected pre-infusion at visits where participants were to receive the study drug.

### Safety Assessments:

The primary safety endpoint was the incidence of TEAEs. A TEAE was defined as any AE with an onset at any time after the first study drug administration.
The secondary safety endpoints included the following:
DLTs; and
The change from baseline in safety assessments (including clinical laboratory assessments, ECGs, vital signs, and physical examinations).

### Adverse Events:

An AE was defined as any untoward medical occurrence in a clinical investigation participant administered a pharmaceutical product, which did not necessarily have a causal relationship with this treatment. An AE could therefore be any unfavourable and/or unintended sign (including an abnormal laboratory finding), symptom, or disease temporally associated with the use of an investigational medicinal product, whether or not related to the investigational medicinal product. All AEs, including observed or volunteered problems, complaints, or symptoms, were recorded on the appropriate eCRF.

AEs, which included clinical laboratory test variables, were monitored and documented from the time of informed consent until study participation was complete. Participants were instructed to report any AE that they experienced to the Investigator, whether or not they thought the event was due to study drug. Following informed consent, Investigators made an assessment for AEs at each visit and recorded the event on the appropriate AE eCRF.

Wherever possible, a specific disease or syndrome rather than individual associated signs and symptoms were identified by the Investigator and recorded on the eCRF. However, if an observed or reported sign or symptom was not considered a component of a specific disease or syndrome by the Investigator, it was recorded as a separate AE on the appropriate eCRF. Additionally, the condition that led to a medical or surgical procedure (e.g., surgery, endoscopy, tooth extraction, or transfusion) was recorded as an AE, not the procedure itself.

Any medical condition already present prior to informed consent was recorded as medical history and not reported as an AE unless the medical condition or signs or symptoms present at baseline worsened in severity, frequency, or seriousness at any time during the study. In this case, it was reported as an AE.

Clinically significant abnormal laboratory or other examination (e.g., ECG, vital sign, and physical examination) findings that were detected during the study or are present after informed consent was obtained and significantly worsen during the study were reported as AEs, as described below. The Investigator exercised his or her medical and scientific judgment in deciding whether an abnormal laboratory finding or other abnormal assessment was clinically significant. Clinically significant abnormal laboratory values occurring during the clinical study were followed until repeat tests returned to normal, stabilize, or were no longer clinically significant.

Abnormal test results that were determined to be an error were not reported as an AE. Laboratory abnormalities or other abnormal clinical findings (e.g., ECG, vital sign, or physical examination abnormalities) were reported as an AE if any of the following were applicable:
If an intervention was required as a result of the abnormality;
If action taken with the study drug was required as a result of the abnormality; or
Based on the clinical judgment of the Investigator.

### Adverse Drug Reaction

All noxious and unintended responses to a medicinal product related to any dose were considered an adverse drug reaction (ADR). “Responses” to a medicinal product meant that a causal relationship between a medicinal product and an AE was at least a reasonable possibility, i.e., the relationship could not be ruled out.

### Unexpected Adverse Drug Reaction:

An Unexpected ADR was defined as an adverse reaction, the nature or severity of which was not consistent with the applicable product information. For budoprutug, the reference safety information was included in the Investigator's Brochure in force at the time of the study. The reference safety information was reviewed annually and the periodicity of the review was harmonized with the reporting period of the Development Safety Update Report.

### Assessment of Adverse Events by the Investigator:

AEs, including those in the context of DLTs, were graded per National Cancer Institute (NCI) CTCAE version 5.0. For those AE terms not listed in the NCI CTCAE, the following grading system was used:
CTCAE Grade 1: Mild; asymptomatic or mild symptoms; clinical or diagnostic observations only; intervention not indicated;
CTCAE Grade 2: Moderate; minimal local or noninvasive intervention indicated; limiting age-appropriate instrumental activities of daily living;
CTCAE Grade 3: Severe or medically significant but not immediately life-threatening; hospitalization or prolongation of hospitalization indicated; disabling; limiting self-care activities of daily living;
CTCAE Grade 4: Life-threatening consequences; urgent intervention indicated; and
CTCAE Grade 5: Death related to the AE.

### Causality Assessment

The relationship of an AE to the administration of the study drug was assessed according to the following definitions:
No (not related, unlikely to be related)—The time course between the administration of study drug and the occurrence or worsening of the AE ruled out a causal relationship and another cause (concomitant drugs, therapies, complications, etc.) was suspected.
Yes (possibly, probably, or definitely related)—The time course between the administration of study drug and the occurrence or worsening of the AE was consistent with a causal relationship and no other cause (concomitant drugs, therapies, complications, etc.) could be identified.

The definition implied a reasonable possibility of a causal relationship between the event and the study drug. This meant that there were facts (evidence) or arguments to suggest a causal relationship.

The following factors were also considered:
The temporal sequence from study drug administration—
The event occurred after the study drug was given. The length of time from study drug exposure to event was evaluated in the clinical context of the event.
Underlying, concomitant, intercurrent diseases—
Each report was evaluated in the context of the natural history and course of the disease being treated and any other disease the participant may have had.
Concomitant drug—other drugs the participant was taking or the treatment the participant received were examined to determine whether any of them might have been recognized to cause the event in question.
Known response pattern for this class of study drug—
Clinical and/or preclinical data indicated whether a particular response was likely to be a class effect.
Exposure to physical and/or mental stresses—
The exposure to stress induced adverse changes in the recipient and provided a logical and better explanation for the event.
The pharmacology and PK of the study drug—
The known pharmacologic properties (absorption, distribution, metabolism, and excretion) of the study drug were considered.

### Adverse Events of Special Interest

The Investigator monitored each participant for clinical and laboratory evidence for pre-defined AESIs throughout the participant's participation in this study. AESIs were chosen on the basis of drug-specific factors as well as disease-specific factors.

The Investigator assessed and recorded any additional information on the AESI in detail on an AE form which was submitted within 24 hours of awareness of the event.

For this study, AESIs included the following:
IRR (any grade);
Severely decreased neutrophil count (i.e., absolute neutrophil count<500/mm³);
Serious infection (i.e., CTCAE Grade 3 or higher);
AKI; and
Thromboembolic events.
   During the course of the study, additional AESIs could have been identified by the Sponsor. AESIs were recorded in the eCRF.

### Serious Adverse Events

An AE or adverse reaction was considered serious if, in the view of either the Investigator or Sponsor, it resulted in any of the following outcomes:
Death;
A life-threatening AE;
Note: An AE or adverse reaction was considered “life-threatening” if, in view of either the Investigator or Sponsor, its occurrence places the participant at immediate risk of death. It did not include an event that, had it occurred in a more severe form, might have caused death.
Required hospitalization or prolongation of existing hospitalizations;
Note: Any hospital admission with at least 1 overnight stay was considered an inpatient hospitalization. An emergency room or urgent care visit without hospital admission was not recorded as an SAE under this criterion, nor was hospitalization for a procedure scheduled or planned before signing of informed consent, or elective treatment of a pre-existing condition that did not worsen from baseline. However, unexpected complications and/or prolongation of hospitalization that occurred during elective surgery were recorded as AEs and assessed for seriousness. Admission to the hospital for social or situational reasons (i.e., no place to stay, live too far away to come for hospital visits, respite care) were not considered inpatient hospitalizations.
A persistent or significant disability/incapacity or substantial disruption of the ability to conduct normal life functions;
A congenital anomaly/birth defect; or
An important medical event.
Note: Important medical events that did not meet any of the above criteria were considered an SAE when, based upon appropriate medical judgment, they may have jeopardize the participant and may have required medical or surgical intervention to prevent one of the outcomes listed above. Examples of such medical events included allergic bronchospasm requiring intensive treatment in an emergency room or at home, blood dyscrasias or convulsions that did not result in inpatient hospitalizations, or the development of drug dependency.

### Analysis Populations:

All treated population (ATP) was defined as the set of participants who received at least 1 dose of study drug.

The ATP was the primary analysis population for summaries of participant characteristics and safety analyses.

### Full-Analysis Set:

The full analysis set (FAS) contained all participants in the ATP, excluding participants who met any of the following conditions:
Participant did not provide informed consent.
Participant did not receive all eligible doses of budoprutug:
   If the participant was recruited under Protocol Versions 1.0 to 5.0. which assigned 4 doses of budoprutug, and the participant did not receive all 4 doses.
   If the participant was recruited under Protocol Version 6.0 (dated 23 Jun. 2023), which assigned 2 doses of budoprutug, and the participant did not receive both doses.

The FAS was the primary analysis population for the analysis of B-cell depletion and other PD endpoints.

### Pharmacokinetic Analysis Set:

The PK population consisted of all participants in the ATP who had sufficient blood samples taken for at least 1 of the PK variables to be calculated.

The PK analysis set was the primary population for all PK analyses.

### Analysis of Subgroups:

Multiple secondary efficacy measures were planned to include participants who were anti-PLA2R antibody positive at baseline only. The following analyses were performed involving this subgroup only:
The change from baseline in anti-PLA2R antibody levels.
The proportion of anti-PLA2R antibody-negative participants (defined as <2 RU/mL) over time.
The proportion of participants achieving ≥50% reduction of serum anti-PLA2R antibody levels from baseline over time.
The proportion of participants achieving >90% reduction of serum anti-PLA2R antibody levels from baseline over time.

### Determination of Sample Size

The sample size was determined empirically to meet the objectives of the study while recognizing the low prevalence of MN that precludes a larger study. Each cohort size was designed to be informative and flexible enough to add participants to a dose cohort if determined necessary by the DRC. Following participants longitudinally for safety, persistence of B-cell depletion, anti-PLA2R antibody reduction, and proteinuria reduction further determined the clinical profile of budoprutug and optimize the learnings from the selected sample size.

The total number of participants depended upon the number of participants accrued to each dose level and the number of dose levels evaluated. It was anticipated that 2 dose cohorts would be evaluated (3 to 6 participants per cohort).

### Participant Disposition

Participant Disposition was summarised using a table and listing. Percentages were calculated using the ATP.

Analysis populations was also summarised using a table and a listing. Percentages were calculated using the ATP.

### Protocol Violations

Protocol Violations were summarised using a table and listing. Percentages were calculated using the number of participants screened.

### Demographics

Demographics were be summarised using a table and listing. Percentages were calculated using the ATP.

### Baseline Disease Characteristics

Baseline disease characteristics were be summarised using a table and listing. Percentages were calculated using the ATP.

### Medical History

Medical history was summarised using a table and listing. Percentages were calculated using the ATP.

### Prior and Concomitant Medications

Prior and concomitant medications were summarised using a table and listing. Percentages were calculated using the ATP.

### Efficacy Measures

The efficacy endpoints included collected and derived continuous and categorical variables.

### Primary Efficacy Analysis

There were no primary efficacy analyses planned for this study.

### Secondary Efficacy Analysis:

### Budoprutug PK Parameters

All bioanalytical and PK data generated were summarised by analyte and treatment using a table and listing. Percentages calculated using the PK population.

Arithmetic mean plasma concentration (±standard deviation [SD]) was presented over time on linear/linear and log 10/linear scales using figures. Spaghetti plots were used to display individual plasma concentrations over time for individual treatment groups using linear/linear scales. Plots were created using the PK population.

### Absolute Peripheral CD20+ B-Cell Count (Cells/μL)

Absolute peripheral CD20+ B-cell count was summarised by visit using a listing. Percentages were calculated using the FAS.

Changes in CD20+ B-cell count from baseline and over time (by visit) were summarised using a table and a patient profiles plot. For both the table and the figure, data were grouped by treatment dose and contained participants in the FAS only.

The proportion of participants achieving a PD response, with >90% B-cell depletion and/or B-cell count<25 cells/μL were summarised using a table. In addition, the proportion of participants with B-cell count<5.02 cells/μL were also summarised on the same table. Proportions were calculated and displayed for all post-baseline visits. Percentages were calculated using the FAS.

Proteinuria (UCPR in g/g)

Proteinuria via UCPR were summarised using a listing. Percentages were calculated using the FAS.

Changes in proteinuria from baseline and over time (by visit) were summarised using a table and a patient profiles plot. For both the table and the figure, data were grouped by treatment dose and contained participants in the FAS only.

The proportion of participants achieving complete remission, partial remission, and a ≥50% reduction in proteinuria when compared to baseline were summarised using a table. Percentages were calculated using the FAS.

Albuminuria (UACR in g/g) and Albuminemia (g/dL)

Albuminuria (via UACR) and albuminemia (via blood albumin) were summarised using a listing. Percentages were calculated using the FAS.

Changes in albuminuria and albuminemia from baseline and over time (by visit) were summarised using a table. Data were grouped by treatment dose and contained participants in the FAS only.

The proportion of participants achieving a ≥50% reduction in albuminuria or ≥25% increase in albuminemia when compared to baseline were summarised using a table. Percentages were calculated using the FAS.

### Nephrotic Syndrome (NS)

Both NS (without edema) and NS (with edema) developing and/or being resolved on-study were summarised using a listing. Percentages were calculated using the FAS.

The proportion of participants developing NS (without edema and with edema) on-study and/or the proportion whose NS resolves on-study were summarised using a table. Percentages were calculated using the FAS.

Serum IgA, IgG, and IgM (Mg/dL or g/L)

Samples for immunoglobulin assessment (Serum IgA, IgG and IgM) were summarised using a listing. Percentages were calculated using the FAS.

The change in serum IgA, IgG and IgM from baseline and over time were summarised using a table and a patient profiles plot. For both the table and the figure, data were grouped by treatment dose and contained participants in the FAS only.

ADA Development any Time after Study Drug Administration

ADA development was summarised using a listing. Percentages was calculated using the FAS.

The incidence of participants who developed ADAs following drug administration were summarised using a table. Percentages were calculated using the FAS.

### Anti-PLA2R Antibody Levels (RU/mL)

Anti-PLA2R antibody levels were summarised using a listing. Percentages were calculated using the FAS.

The anti-PLA2R change from baseline and over time were summarised using a table and a patient profiles plot. For both the table and the figure, data were grouped by treatment dose and will contain participants in the FAS who were anti-PLA2R positive at baseline only.

The proportion of anti-PLA2R antibody negative participants over time (by visit), as well as the proportion of participants achieving a ≥50% and/or ≥90% reduction in anti-PLA2R antibodies were summarised using tables. Proportions were presented for each of the visits.

Percentages were calculated using participants in FAS who were anti-PLA2R positive at baseline.

### Exploratory Efficacy Analyses:

### Change in Participant Reported QOL and Edema Outcomes

Self-reported outcomes relating to QOL and edema were summarised using a listing. Percentages were calculated using participants in the FAS.

Changes in self-reported outcomes over time were summarised using a table. Percentages were calculated using participants in the FAS.

### The Relationship Between PK, B-Cell Depletion and Clinical Response

The relationship between PK vs B-cell depletion and clinical response were presented using a time series plot (by visit). The figure were created using the FAS.

### The Relationship Between Treatment Dose, B-Cell Depletion and Clinical Response

The relationship between PK vs B-cell depletion and clinical response were presented using a time series plot (by visit). The figure was created using the FAS.

### Adverse Events:

Adverse Events were summarised using tables and listings. Percentages were calculated using the ATP.

The following types of AEs were summarised:
Overview of all AEs.
Treatment-Emergent Adverse Events.
Adverse Events Related to Study Treatment.
Serious Adverse Events.
Adverse Events by CTCAE Grading.
Adverse Events Leading to Dose Interruption/Reduction.
Adverse Events Leading to Treatment Discontinuation.
Adverse Events of Special Interest.

### Changes to the Planned Analysis:

The most recent study protocol available at the time of writing the Statistical Analysis Plan (SAP) was Protocol Version 6.0.0 (dated 23 Jun. 2023). However, this protocol amendment was made late into the lifespan of the study, by which point the majority of the data had already been collected. Differences between Protocol were mostly visit-related and treatment dose-related. Following discussion with the sponsor, it was decided to write the SAP based on Protocol Version 6.0, but following the visit schedule and treatment doses described in the previous Protocol, which allowed for analysis of all data collected during the study.

The following changes to planned analysis were made:
For Visit 3 and Visit 5 no actual sample collection times were available and therefore a nominal time of 168 h was assumed and used in the PK analysis
Exposure accumulation ratios (maximum observed plasma drug concentration [Cₘₐₓ] and AUCₗₐₛₜ) were also calculated where possible
Participants were dosed either a cumulative 200 mg or 400 mg IV dose of budoprutug.

### Study Participants:

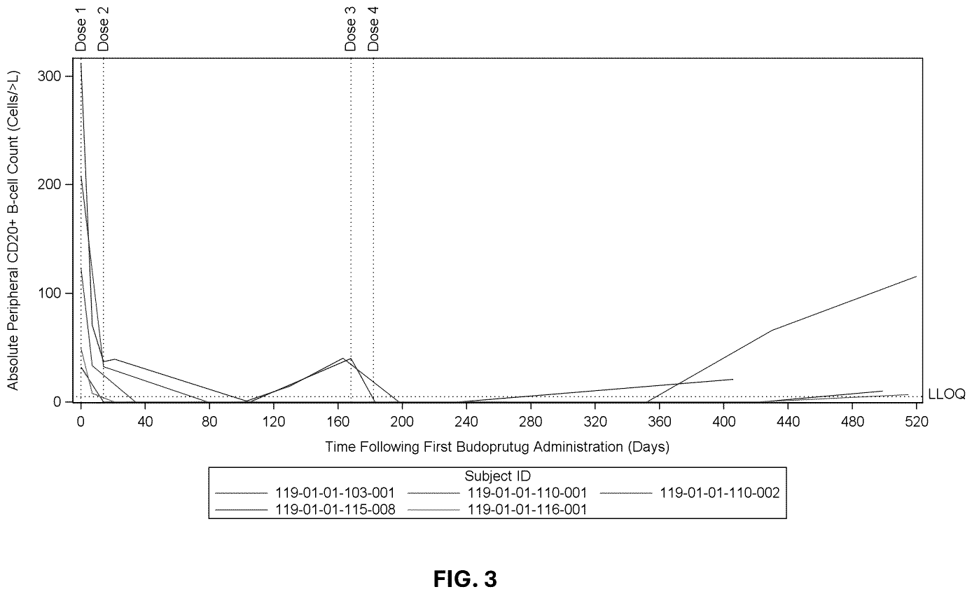

Of the 38 participants who were screened, 8 participants were enrolled in the study. All enrolled participants received budoprutug (4 in the budoprutug 100 mg Cohort and 4 in the budoprutug 200 mg Cohort). Overall, 5 (62.5%) participants received at least 4 doses of budoprutug and had at least 48 weeks of data. Three participants completed the study (2 [50%] in the budoprutug 100 mg Cohort and 1 [25%] in the budoprutug 200 mg Cohort). FIG. 2 provides a summary of the participants in the study.

### Protocol Deviations:

Summary of protocol violations for screened participants are provided in the table below.

| | Total |
|---|---|
| | (N = 38)^{a} |
| Number of Participants with at least one Protocol | 12 (31.6%) |
| Violation | |
| Violation Category | |
| Exclusion Criteria | 5 (13.2%) |
| Inclusion Criteria | 1 (2.6%) |
| Informed Consent | 1 (2.6%) |
| Investigational Product | 1 (2.6%) |
| Investigational Product (other than incorrect dose or | 1 (2.6%) |
| wrong treatment) | |
| Study Procedures - CSR Non-Reportable | 8 (21.1%) |
| Study Procedures Impacting Primary or Secondary | 15 (39.5%) |
| Endpoint | |
| Participant not consented under most recent version of | 1 (2.6%) |
| the ICF (specify version) | |
| Visit Window | 13 (34.2%) |
| Visit Window - CSR Non-Reportable | 8 (21.1%) |

### Data Sets Analyzed

All 8 treated participants were included in the ATP and PK populations. The FAS included 5 (62.5%). The Table below summarizes analysis sets by treatment group.

| | 100 mg | 200 mg | Total |
|---|---|---|---|
| | (N = 4) | (N = 4) | (N = 8) |
| Number of Participants in the ATP | 4 (100%) | 4 (100%) | 8 (100%) |
| Number of Participants in the FAS | 3 (75.0%) | 2 (50.0%) | 5 (62.5%) |
| Number of Participants in the PK | 4 (100%) | 4 (100%) | 8 (100%) |
| Population | | | |
| Number of Participants Excluded | | | |
| from the FAS | | | |
| Participant did not receive all | 1 (25.0%) | 2 (50.0%) | 3 (37.5%) |
| eligible doses of Budoprutug | | | |

### Demographics:

The mean (SD) age was 49.8 years old (14.68) with a mean (SD) body mass index of 30.6 kg/m² (10.2). The majority of participants were male and White. The demographic data were similar across treatment groups. The Table below summarizes demography and baseline characteristics by treatment group for the ATP.

TABLE
Summary of Demography and Baseline Characteristics:
All Treated Population

| | 100 mg | 200 mg | Total |
|---|---|---|---|
| | (N = 4) | (N = 4) | (N = 8) |
| Age (years) | | | |
| n | 4 | 4 | 8 |
| Mean | 53.3 | 46.3 | 49.8 |
| SD | 12.71 | 17.56 | 14.68 |
| Median | 57.0 | 49.5 | 52.5 |
| Minimum | 35 | 22 | 22 |
| Maximum | 64 | 64 | 64 |
| Gender | | | |
| Male | 3 (75.0%) | 4 (100.0%) | 7 (87.5%) |
| Female | 1 (25.0%) | 0 | 1 (12.5%) |
| Race | | | |
| White | 4 (100.0%) | 3 (75.0%) | 7 (87.5%) |
| Black or African American | 0 | 1 (25.0%) | 1 (12.5%) |
| Asian | 0 | 0 | 0 |
| American Indian or | 0 | 0 | 0 |
| Alaskan Native | | | |
| Native Hawaiian or other | 0 | 0 | 0 |
| Pacific Islander | | | |
| Ethnicity | | | |
| Hispanic or Latino | 2 (50.0%) | 1 (25.0%) | 3 (37.5%) |
| Not Hispanic or Latino | 2 (50.0%) | 3 (75.0%) | 5 (62.5%) |

The Table below provides the baseline disease characteristics:

### Summary of Prior Disease History: All Treated Population

| | 100 mg | 200 mg | Total |
|---|---|---|---|
| | (N = 4) | (N = 4) | (N = 8) |
| Participants with Disease History | | | |
| Type | | | |
| Renal Biopsy | 3 (75.0%) | 4 (100%) | 7 (87.5%) |
| eGFR | 0 | 0 | 0 |
| Anti-PLA2R Antibody Titers | 0 | 1 (25.0%) | 1 (12.5%) |
| Proteinuria | 0 | 1 (25.0%) | 1 (12.5%) |
| B-Cell/Lymphocyte Levels | 0 | 0 | 0 |
| MN Treatment Received | 1 (25.0%) | 2 (50.0%) | 3 (37.5%) |
| Renal Biopsy Sample Result for | | | |
| PLA2R | | | |
| Positive | 1 (25.0%) | 3 (75.0%) | 4 (50.0%) |
| Negative | 1 (25.0%) | 1 (25.0%) | 2 (25.0%) |
| Missing | 2 (50.0%) | 0 | 2 (25.0%) |
| Renal biopsy sample used for | | | |
| diagnosis of membranous | | | |
| nephropathy | | | |
| No | 0 | 0 | 0 |
| Yes | 2 (50.0%) | 4 (100%) | 6 (75.0%) |
| Missing | 2 (50.0%) | 0 | 2 (25.0%) |
| Evidence of FSGS | | | |
| Yes | 0 | 1 (25.0%) | 1 (12.5%) |
| Primary | 0 | 0 | 0 |
| Secondary | 0 | 1 (25.0%) | 1 (12.5%) |
| Other | 0 | 0 | 0 |
| No | 3 (75.0%) | 3 (75.0%) | 6 (75.0%) |
| Missing | 1 (25.0%) | 0 | 1 (12.5%) |
| Any Other Historical Diagnoses | | | |
| No | 2 (50.0%) | 3 (75.0%) | 5 (62.5%) |
| Yes | 1 (25.0%) | 1 (25.0%) | 2 (25.0%) |
| Missing | 1 (25.0%) | 0 | 1 (12.5%) |
| Presence of Tubulointerstitial or | | | |
| Interstitial Fibrosis | | | |
| Yes | 0 | 1 (25.0%) | 1 (12.5%) |
| Mild (0-25%) | 0 | 1 (25.0%) | 1 (12.5%) |
| Moderate (>25%-50%) | 0 | 0 | 0 |
| Severe (>50%) | 0 | 0 | 0 |
| No | 1 (25.0%) | 0 | 1 (12.5%) |
| Missing | 3 (75.0%) | 3 (75.0%) | 6 (75.0%) |
| Anti-PLA2R antibody titers | | | |
| Positive | 0 | 1 (25.0%) | 1 (12.5%) |
| Negative | 0 | 0 | 0 |
| Missing | 4 (100%) | 3 (75.0%) | 7 (87.5%) |
| Anti-PLA2R antibody titre value | | | |
| (RU/mL) | | | |
| n | | 1 | 1 |
| Mean | | 4.0 | 4.0 |
| Median | | 4.0 | 4.0 |
| SD | | | |
| Minimum | | 4 | 4 |
| Maximum | | 4 | 4 |
| Proteinuria | | | |
| No | 0 | 0 | 0 |
| Yes | 0 | 1 (25.0%) | 1 (12.5%) |
| Missing | 4 (100%) | 3 (75.0%) | 7 (87.5%) |
| Proteinuria (UPCR in g/g) | | | |
| n | | 1 | 1 |
| Mean | | 6.20 | 6.20 |
| Median | | 6.20 | 6.20 |
| SD | | | |
| Minimum | | 6.2 | 6.2 |
| Maximum | | 6.2 | 6.2 |
| 24-Hour Urine Collection (mg/24 | | | |
| hours) | | | |
| n | | | |
| Missing | | 1 | 1 |
| Mean | | | |
| Median | | | |
| SD | | | |
| Minimum | | | |
| Maximum | | | |
| MN treatment received | | | |
| B-Cell Depleting Therapy | 1 (25.0%) | 0 | 1 (12.5%) |
| Glucocorticoids | 0 | 1 (25.0%) | 1 (12.5%) |
| Alkylating Agent (e.g., | 0 | 0 | 0 |
| Cyclophosphamide) | | | |
| Calcineurin Inhibitor (e.g., | 0 | 1 (25.0%) | 1 (12.5%) |
| Cyclosporin or Tacrolimus) | | | |
| Mycophenolate Mofetil/Sodium | 0 | 0 | 0 |
| Azathioprine | 0 | 0 | 0 |
| Other | 0 | 0 | 0 |
| Missing | 3 (75.0%) | 2 (50.0%) | 5 (62.5%) |

Abbreviation: eGFR = estimated glomerular filtration rate; MN = membranous nephropathy; N/n = number of participants; SD = standard deviation; PLA2R = phospholipase A2 receptor; UPCR = urine protein-to-creatinine ratio.

### Previous and Concomitant Medication:

All participants were at least on 1 prior and/or concomitant medication. The most commonly reported prior medications were lisinopril reported by 5 (63.0%) participants and atorvastatin reported by 4 (50.0%) participants. The most commonly reported concomitant medications were paracetamol reported by 8 (100%) participants and diphenhydramine, cetirizine, and methylprednisolone reported by 4 (50.0%) participants each.

### Extent of Exposure:

The entire dose was administered in 8 (100%), 7 (87.5%), 5 (62.5%), and 5 (62.5%) participants at Week 1, Week 3, Week 24, and Week 26 visits, respectively. Overall, 5 (62.5%) participants received at least 4 doses of budoprutug and had at least 48 weeks of data.

### Efficacy, Pharmacokinetic, Pharmacodynamic, and Other Evaluations:

Mean (SD) percentage change from baseline at the EOS was −78.4 (22.3; −74.6 [26.3] for participants in budoprutug 100 mg group and −84.2 [22.3] for participants in budoprutug 200 mg group). The proportion (95% confidence interval [CI]) of participants achieving ≥90% B-cell depletion was 0.4 (0.118, 0.769). The proportion (95% CI) of participants achieving B-cell count of <5.02 cells/μL was 0.2 (0.036, 0.624).

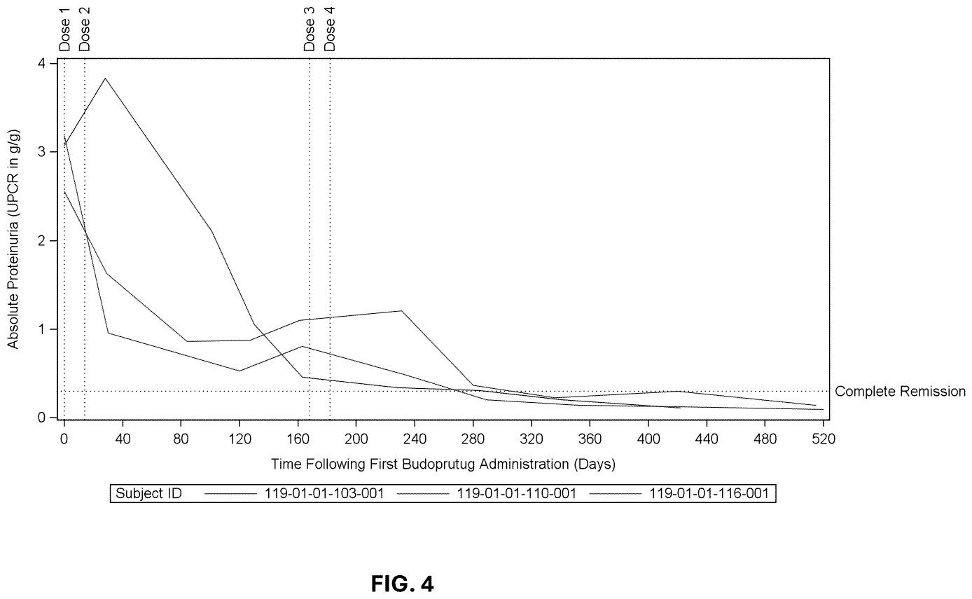

FIG. 3 shows the average peripheral CD20+ B-cell count over time following first budoprutug administration for participants with MN for the FAS. Average peripheral CD20+ B-cell count overtime following first budoprutug administration for participants with MN for the FAS are also measured.

Proteinuria (UPCR), Albuminuria (UACR), and Albuminemia (g/dL)

### Proteinuria (UPCR Assessments)

Mean (SD) change from baseline in UPCR at the end of the study was −2.8 (0.48) g/g. All participants achieved complete remission. Thus the proportion (95% CI) of participants achieving ≥50% reduction of proteinuria from baseline was also 1 (0.342, 1).

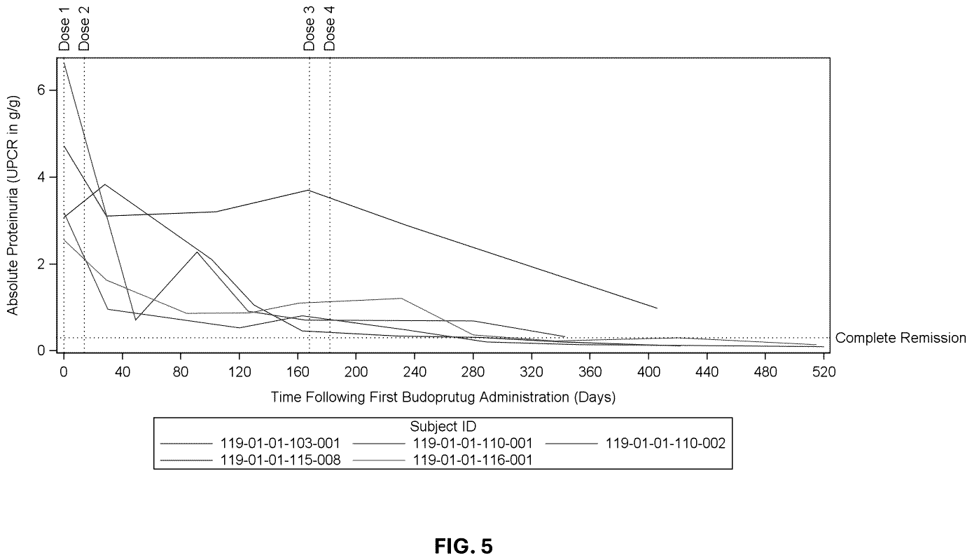

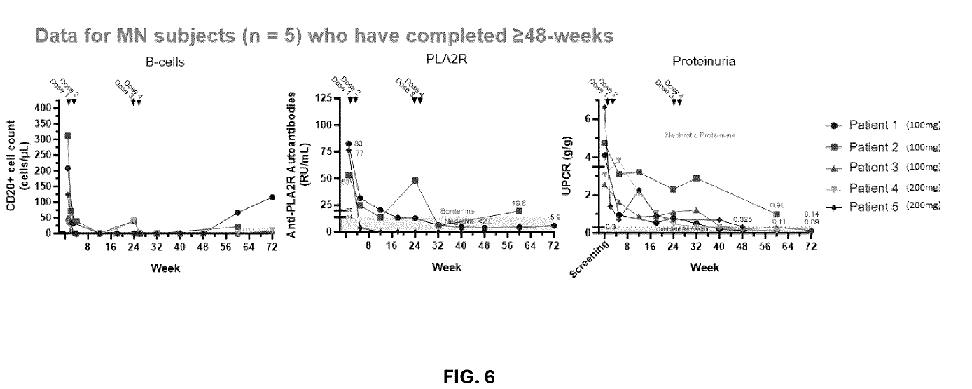

FIG. 4 shows the average proteinuria (UPCR) over time following first budoprutug administration for participants with MN for the FAS and FIG. 5 shows the sensitivity analysis. The baseline value at Day 0 is defined as the mean of Screening Visit and Qualifying Visit. Complete Remission was defined as UPCR≤0.3 g/g. Additionally including participants who obtained 2 consecutive UPCR measurements ≥2.0 within 21 days during the Screening Period.

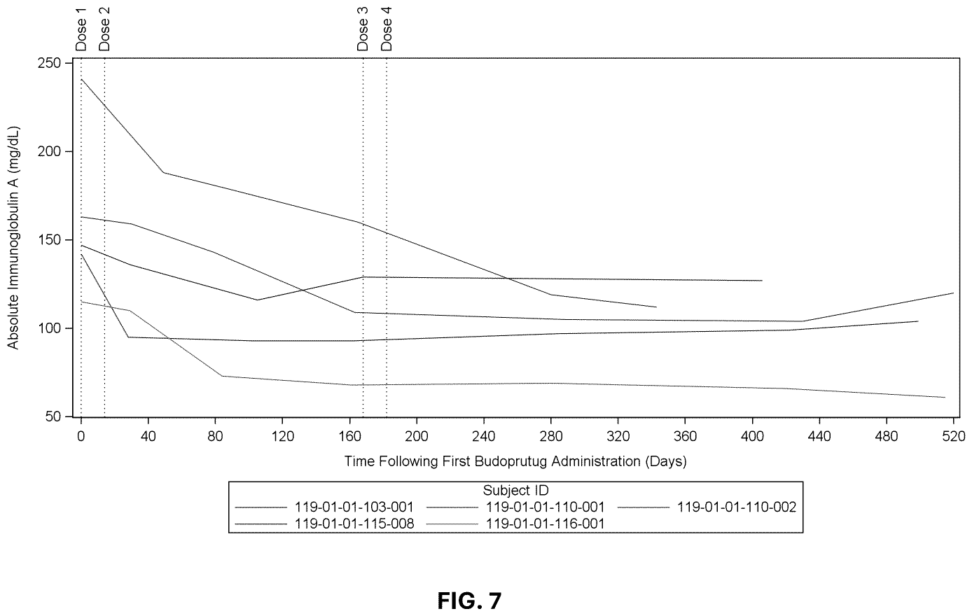

FIG. 6 provides collated data for the CD20+ cell count, anti-PLA2R antibodies, and UPCR at various time points after the administration of budoprutug. The data provided in FIG. 6 demonstrates that complete remission was achieved in 60% (3/5) of patients at Week 48.
Partial remission (>50% reduction in UPCR+UPCR<3.5 g/g) achieved in all (5/5) subjects.
Complete B-cell depletion (CD20⁺ count<5 cells/μL) achieved in all (5/5) subjects
Anti-PLA2R Ab negativity (<14 RU/mL) achieved in all (3/3) evaluable subjects
2 subjects on study that have not entered complete remission have achieved PLA2R negativity (serological remission)

In effect, budoprutug in MN was well-tolerated and demonstrated clinical and immunological remission rates superior to rituximab based on historical data.

### Immunoglobulin Assessments:

Mean (SD) change in serum IgA, IgG, and IgM from baseline over time were-56.8 mg/dL (42.19), 154.2 mg/dL (114.84), and −13.6 mg/dL (5.98), respectively.

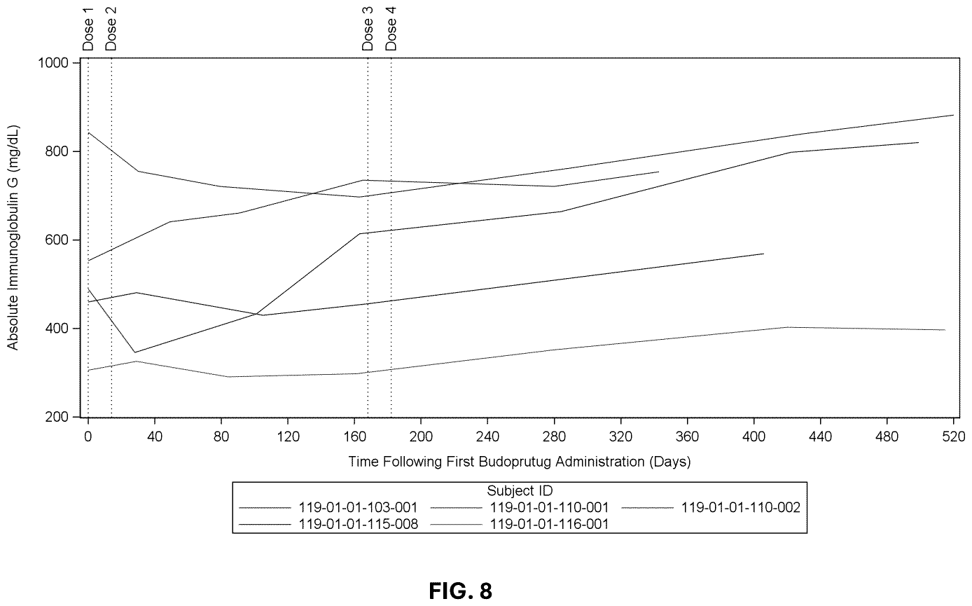

FIG. 7, FIG. 8, and FIG. 9 show the IgA, IgG, and IgM versus time following first budoprutug administration for participants with MN for the FAS, respectively.

### Anti-PLA2R Antibodies:

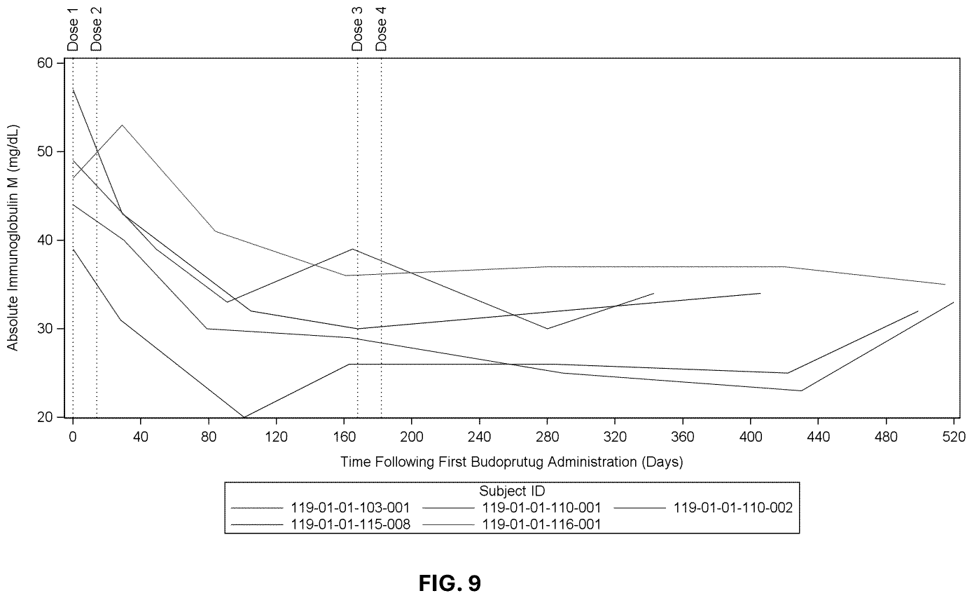

Participant anti-PLA2R antibody levels following budoprutug administration. Mean (SD) change from baseline in anti-PLA2R antibody levels at EOS was-62.1 RU/mL (24.9). FIG. 10 shows average anti-PLA2R antibodies over time. The proportion (95% CI) of anti-PLA2R negative participants was 0.7 (0.2, 0.9) at the end of the study. The proportion (95% CI) of participants achieving a ≥50% and a ≥90% reduction in anti-PLA2R antibodies were 1 (0.4, 1) and 0.7 (0.2, 0.9), respectively.

### Examination of Subgroups:

Multiple secondary efficacy measures included participants who were anti-PLA2R antibody positive at baseline only. The following analyses were performed involving this subgroup only:
The change from baseline in anti-PLA2R antibody levels.
The proportion of anti-PLA2R antibody-negative participants (defined as <2 RU/mL) over time.
The proportion of participants achieving ≥50% reduction of serum anti-PLA2R antibody levels from baseline over time.
The proportion of participants achieving ≥90% reduction of serum anti-PLA2R antibody levels from baseline over time.

### Plasma Concentration Over Time:

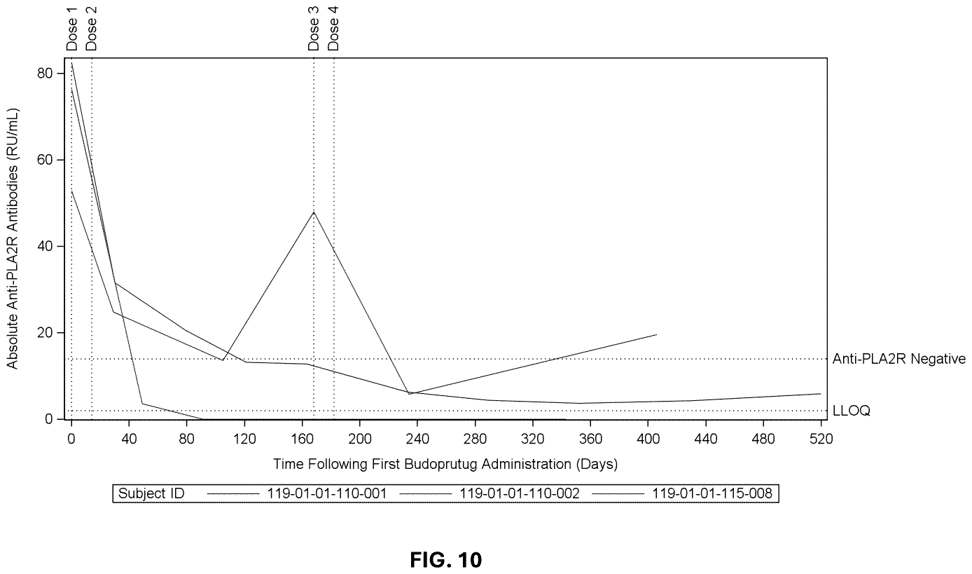

FIG. 11 shows plasma budoprutug concentration versus time for the PK population.

### Efficacy:

A decrease in absolute peripheral CD20+ B-Cell was observed from baseline for both treatment groups. The decrease was greater for the budoprutug 200 mg group.
There was a sustained decrease in proteinuria as measured by UPCR throughout the treatment period with all participants achieving complete remission (proteinuria≤0.3 g/g).
There was a reduction in serum IgA and IgM from baseline. The reduction was greater with budoprutug 200 mg.

### Pharmacokinetics:

Following IV infusion of 100 or 200 mg (cumulative 200 mg or 400 mg) of budoprutug to adult participants with primary membranous nephropathy the exposure appeared less than proportional upon doubling the dose.
The serum half-life was considered long at 405.80 h and 219.97 h following dosing of 100 mg and 200 mg budoprutug, respectively after the Day 15 dose.
The serum clearance was considered low at <0.99 L/day.
No considerable accumulation (as determined by Cₘₐₓ) was observed following dosing at 100 mg and 200 mg budoprutug (0.90 and 1.10, respectively).
Overall, there was a considerable amount of variability observed in the determined PK parameters across 2 doses and dosing periods.

### Safety:

Summary of AEs Following budoprutug administration for participants with MN for ATP is presented in the Table below.

Overall, 7 (87.5%) participants reported at least one TEAE. Of these, 4 (50.0%) participants had at least 1 treatment-related TEAE. There were 3 (37.5%) participants with at least one SAE, 1 (12.5%) participant with an ADR, and 3 (37.5%) participants with an AESI. There were no study discontinuations due to an AE. No DLTs were reported during the conduct of this study.

TABLE
Overview of AEs Following Budoprutug Administration for Participants with MN:
All Treated Population

| | 100 mg | | 200 mg | | Total | |
|---|---|---|---|---|---|---|
| | (N = 4) | | (N = 4) | | (N = 8) | |
| | n (%) | Event | n (%) | Event | n (%) | Event |
| Number of Participants/Events with at | 3 (75.0%) | 15 | 4 (100%) | 14 | 7 (87.5%) | 29 |
| Least One AE | | | | | | |
| Number of Participants/Events with at | 3 (75.0%) | 15 | 4 (100%) | 14 | 7 (87.5%) | 29 |
| Least One TEAE | | | | | | |
| Number of Participants/Events with at | 2 (50.0%) | 4 | 2 (50.0%) | 3 | 4 (50.0%) | 7 |
| Least One Treatment-Related TEAE | | | | | | |
| Number of Participants/Events with at | 1 (25.0%) | 1 | 2 (50.0%) | 2 | 3 (37.5%) | 3 |
| Least One Serious TEAE | | | | | | |
| Number of Participants/Events with at | 1 (25.0%) | 1 | 2 (50.0%) | 3 | 3 (37.5%) | 4 |
| Least One TEAE with NCI-CTCAE | | | | | | |
| Grade 3 or Higher | | | | | | |
| Number of Participants/Events with at | 0 | 0 | 1 (25.0%) | 2 | 1 (12.5%) | 2 |
| Least One ADR | | | | | | |
| Number of Participants/Events with at | 0 | 0 | 0 | 0 | 0 | 0 |
| Least One Unexpected Adverse Drug | | | | | | |
| Reaction | | | | | | |
| Number of Participants/Events with at | 2 (50.0%) | 2 | 1 (25.0%) | 2 | 3 (37.5%) | 4 |
| Least One AESI | | | | | | |
| Number of Participants/Events with at | 0 | 0 | 0 | 0 | 0 | 0 |
| Least One DLT | | | | | | |
| Number of Participants with at Least | | | | | | |
| One AEs Leading To: | | | | | | |
| Dose Not Changed | 3 (75.0%) | 13 | 4 (100%) | 11 | 7 (87.5%) | 24 |
| Dose Interruption | 0 | 0 | 1 (25.0%) | 1 | 1 (12.5%) | 1 |
| Not Applicable | 1 (25.0%) | 2 | 2 (50.0%) | 2 | 3 (37.5%) | 4 |
| Number of Participants with an AE | 0 | 0 | 0 | 0 | 0 | 0 |
| Leading to Study Discontinuation | | | | | | |
| Number of Participants with an AE | 0 | 0 | 0 | 0 | 0 | 0 |
| Leading to Death | | | | | | |

Abbreviation: AE = adverse event; N/n = number of participants; AESI = adverse event of specia linterest; DLT = dose-limiting toxicity; NCI-CTCAE = National Cancer Institute Common Terminology Criteria for Adverse Events; TEAE = treatment-emergent adverse event.

### Display of Adverse Events:

The most commonly reported SOC was general disorders and administration site conditions reported by 4 (50.0%) participants followed by infections and infestations reported by 4 (50.0%) participants. The most commonly reported TEAEs by PT were COVID-19 reported by 3 (37.5%) participants and chills reported by 2 (25.0%) participants. The pattern and incidence of TEAEs (by SOC and PT) were similar between treatment groups.

Summary of TEAEs following budoprutug administration for participants with MN by SOC and PT for the ATP is presented in the Table below.

TABLE
Summary of TEAEs Following Budoprutug
Administration for Participants with
MN by SOC and PT: All Treated Population

| SOC | 100 mg | 200 mg | Total |
|---|---|---|---|
| PT | (N = 4) | (N = 4) | (N = 8) |
| Number of Participants With at Least | 3 (75.0%) | 4 (100%) | 7 (87.5%) |
| One TEAE | | | |
| General disorders and administration | 3 (75.0%) | 1 (25.0%) | 4 (50.0%) |
| site conditions | | | |
| Chills | 1 (25.0%) | 1 (25.0%) | 2 (25.0%) |
| Oedema peripheral | 1 (25.0%) | 0 | 1 (12.5%) |
| Peripheral swelling | 1 (25.0%) | 0 | 1 (12.5%) |
| Infections and infestations | 2 (50.0%) | 2 (50.0%) | 4 (50.0%) |
| COVID-19 | 1 (25.0%) | 2 (50.0%) | 3 (37.5%) |
| Conjunctivitis | 0 | 1 (25.0%) | 1 (12.5%) |
| Pneumonia bacterial | 1 (25.0%) | 0 | 1 (12.5%) |
| Upper respiratory tract infection | 0 | 1 (25.0%) | 1 (12.5%) |
| Musculoskeletal and connective | 2 (50.0%) | 1 (25.0%) | 3 (37.5%) |
| tissue disorders | | | |
| Myalgia | 1 (25.0%) | 0 | 1 (12.5%) |
| Pain in extremity | 1 (25.0%) | 0 | 1 (12.5%) |
| Rhabdomyolysis | 0 | 1 (25.0%) | 1 (12.5%) |
| Investigations | 1 (25.0%) | 1 (25.0%) | 2 (25.0%) |
| Blood creatine phosphokinase | 0 | 1 (25.0%) | 1 (12.5%) |
| increased | | | |
| Blood pressure decreased | 1 (25.0%) | 0 | 1 (12.5%) |
| Nervous system disorders | 2 (50.0%) | 0 | 2 (25.0%) |
| Headache | 1 (25.0%) | 0 | 1 (12.5%) |
| Somnolence | 1 (25.0%) | 0 | 1 (12.5%) |
| Renal and urinary disorders | 1 (25.0%) | 1 (25.0%) | 2 (25.0%) |
| Acute kidney injury | 1 (25.0%) | 0 | 1 (12.5%) |
| Haematuria | 0 | 1 (25.0%) | 1 (12.5%) |
| Haemorrhage urinary tract | 0 | 1 (25.0%) | 1 (12.5%) |
| Respiratory, thoracic and | 0 | 2 (50.0%) | 2 (25.0%) |
| mediastinal disorders | | | |
| Chronic obstructive pulmonary | 0 | 1 (25.0%) | 1 (12.5%) |
| disease | | | |
| Cough | 0 | 1 (25.0%) | 1 (12.5%) |
| Cardiac disorders | 1 (25.0%) | 0 | 1 (12.5%) |
| Sinus tachycardia | 1 (25.0%) | 0 | 1 (12.5%) |
| Gastrointestinal disorders | 0 | 1 (25.0%) | 1 (12.5%) |
| Nausea | 0 | 1 (25.0%) | 1 (12.5%) |
| Vomiting | 0 | 1 (25.0%) | 1 (12.5%) |
| Injury, poisoning and procedural | 1 (25.0%) | 0 | 1 (12.5%) |
| complications | | | |
| Lip injury | 1 (25.0%) | 0 | 1 (12.5%) |
| Psychiatric disorders | 0 | 1 (25.0%) | 1 (12.5%) |
| Depression | 0 | 1 (25.0%) | 1 (12.5%) |
| Skin and subcutaneous tissue | 1 (25.0%) | 0 | 1 (12.5%) |
| disorders | | | |
| Angioedema | 1 (25.0%) | 0 | 1 (12.5%) |
| Vascular disorders | 1 (25.0%) | 0 | 1 (12.5%) |
| Hot flush | 1 (25.0%) | 0 | 1 (12.5%) |

All TEAEs are coded using MedDRA Version 24.0.
Each participant is counted only once within each SOC and PT.
Percentages are calculated using the number of participants in the corresponding treatment group as the denominator.
Abbreviations: MedDRA = Medical Dictionary for Regulatory Activities; N = number of participants; MN = membranous neuropathy; N = number of participants; PT = Preferred Term; SOC = System Organ Class; TEAE = treatment-emergent adverse event.

### Analysis of Adverse Events:

### Treatment-Related Treatment-Emergent Adverse Events

There were 4 (50.0%) participants with at least one treatment-related TEAE. The most commonly reported treatment-related TEAE was COVID-19 reported by 2 (25.0%) participants. Summary of TEAEs following budoprutug administration for participants with MN by relationship to study drug for the ATP is presented in the Table below.

Summary of TEAEs Following Budoprutug Administration for Participants with MN by Relationship to Study Drug: All Treated Population

| | 100 mg | | 200 mg | | Total | |
|---|---|---|---|---|---|---|
| | (N = 4) | | (N = 4) | | (N = 8) | |
| System Organ Class | | Not- | | Not- | | Not- |
| Preferred Term | Related | Related | Related | Related | Related | Related |
| Number of Participants With at | 2 (50.0%) | 1 (25.0%) | 2 (50.0%) | 2 (50.0%) | 4 (50.0%) | 3 (37.5%) |
| Least One TEAE | | | | | | |
| General disorders and | 1 (25.0%) | 2 (50.0%) | 0 | 1 (25.0%) | 1 (12.5%) | 3 (37.5%) |
| administration site conditions | | | | | | |
| Chills | 1 (25.0%) | 0 | 0 | 1 (25.0%) | 1 (12.5%) | 1 (12.5%) |
| Oedema peripheral | 0 | 1 (25.0%) | 0 | 0 | 0 | 1 (12.5%) |
| Peripheral swelling | 0 | 1 (25.0%) | 0 | 0 | 0 | 1 (12.5%) |
| Infections and infestations | 1 (25.0%) | 1 (25.0%) | 1 (25.0%) | 1 (25.0%) | 2 (25.0%) | 2 (25.0%) |
| COVID-19 | 1 (25.0%) | 0 | 1 (25.0%) | 1 (25.0%) | 2 (25.0%) | 1 (12.5%) |
| Conjunctivitis | 0 | 0 | 0 | 1 (25.0%) | 0 | 1 (12.5%) |
| Pneumonia bacterial | 0 | 1 (25.0%) | 0 | 0 | 0 | 1 (12.5%) |
| Upper respiratory tract | 0 | 0 | 0 | 1 (25.0%) | 0 | 1 (12.5%) |
| infection | | | | | | |
| Musculoskeletal and connectivel | (25.0%) | 1 (25.0%) | 0 | 1 (25.0%) | 1 (12.5%) | 2 (25.0%) |
| tissue disorders | | | | | | |
| Myalgia | 1 (25.0%) | 0 | 0 | 0 | 1 (12.5%) | 0 |
| Pain in extremity | 0 | 1 (25.0%) | 0 | 0 | 0 | 1 (12.5%) |
| Rhabdomyolysis | 0 | 0 | 0 | 1 (25.0%) | 0 | 1 (12.5%) |
| Investigations | 0 | 1 (25.0%) | 0 | 1 (25.0%) | 0 | 2 (25.0%) |
| Blood creatine phosphokinase | 0 | 0 | 0 | 1 (25.0%) | 0 | 1 (12.5%) |
| increased | | | | | | |
| Blood pressure decreased | 0 | 1 (25.0%) | 0 | 0 | 0 | 1 (12.5%) |
| Nervous system disorders | 0 | 2 (50.0%) | 0 | 0 | 0 | 2 (25.0%) |
| Headache | 0 | 1 (25.0%) | 0 | 0 | 0 | 1 (12.5%) |
| Somnolence | 0 | 1 (25.0%) | 0 | 0 | 0 | 1 (12.5%) |
| Renal and urinary disorders | 0 | 1 (25.0%) | 0 | 1 (25.0%) | 0 | 2 (25.0%) |
| Acute kidney injury | 0 | 1 (25.0%) | 0 | 0 | 0 | 1 (12.5%) |
| Haematuria | 0 | 0 | 0 | 1 (25.0%) | 0 | 1 (12.5%) |
| Haemorrhage urinary tract | 0 | 0 | 0 | 1 (25.0%) | 0 | 1 (12.5%) |
| Respiratory, thoracic and | 0 | 0 | 0 | 2 (50.0%) | 0 | 2 (25.0%) |
| mediastinal disorders | | | | | | |
| Chronic obstructive | 0 | 0 | 0 | 1 (25.0%) | 0 | 1 (12.5%) |
| pulmonary disease | | | | | | |
| Cough | 0 | 0 | 0 | 1 (25.0%) | 0 | 1 (12.5%) |
| Cardiac disorders | 0 | 1 (25.0%) | 0 | 0 | 0 | 1 (12.5%) |
| Sinus tachycardia | 0 | 1 (25.0%) | 0 | 0 | 0 | 1 (12.5%) |
| Gastrointestinal disorders | 0 | 0 | 1 (25.0%) | 0 | 1 (12.5%) | 0 |
| Nausea | 0 | 0 | 1 (25.0%) | 10 | 1 (12.5%) | 0 |
| Vomiting | 0 | 0 | 1 (25.0%) | 0 | 1 (12.5%) | 0 |
| Injury, poisoning and | 0 | 1 (25.0%) | 0 | 0 | 0 | 1 (12.5%) |
| procedural complications | | | | | | |
| Lip injury | 0 | 1 (25.0%) | 0 | 0 | 0 | 1 (12.5%) |
| Psychiatric disorders | 0 | 0 | 0 | 1 (25.0%) | 0 | 1 (12.5%) |
| Depression | 0 | 0 | 0 | 1 (25.0%) | 0 | 1 (12.5%) |
| Skin and subcutaneous tissue | 0 | 1 (25.0%) | 0 | 0 | 0 | 1 (12.5%) |
| disorders | | | | | | |
| Angioedema | 0 | 1 (25.0%) | 0 | 0 | 0 | 1 (12.5%) |
| Vascular disorders | 1 (25.0%) | 0 | 0 | 0 | 1 (12.5%) | 0 |
| Hot flush | 1 (25.0%) | 0 | 0 | 0 | 1 (12.5%) | 0 |

All TEAEs are coded using MedDRA Version 24.0.
Each participant is counted only once within each System Organ Class and Preferred Term using the event that falls into the most severe ‘Related’ category.
An AE is defined as ‘Related’, if it is recorded as ‘Possibly’, ‘Probably’ or ‘Definitely’ related to the study treatment.
Percentages are calculated using the number of participants in the corresponding treatment group as the denominator.
MN: Membranous Neuropathy;
AE: Abbreviations: MedDRA = Medical Dictionary for Regulatory Activities; N = number of participants; MN = membranous neuropathy; N = number of participants; TEAE = treatment-emergent adverse event.

### Serious Adverse Events:

There were 3 (37.5%) participants with treatment-emergent SAEs (pneumonia bacterial, rhabdomyolysis, and chronic obstructive pulmonary disease [COPD]). Summary of serious TEAEs following budoprutug administration for participants with MN by SOC and PT for the ATP is presented in the Table below.

TABLE
Summary of Serious TEAEs Following Budoprutug Administration for
Participants with MN by SOC and PT: All Treated Population

| System Organ Class | 100 mg | 200 mg | Total |
|---|---|---|---|
| Preferred Term | (N = 4) | (N = 4) | (N = 8) |
| Number of Participants With at | 1 (25.0%) | 2 (50.0%) | 3 (37.5%) |
| Least One TEAE | | | |
| Infections and infestations | 1 (25.0%) | 0 | 1 (12.5%) |
| Pneumonia bacterial | 1 (25.0%) | 0 | 1 (12.5%) |
| Musculoskeletal and connective | 0 | 1 (25.0%) | 1 (12.5%) |
| tissue disorders | | | |
| Rhabdomyolysis | 0 | 1 (25.0%) | 1 (12.5%) |
| Respiratory, thoracic and mediastinal | 0 | 1 (25.0%) | 1 (12.5%) |
| disorders | | | |
| Chronic obstructive pulmonary | 0 | 1 (25.0%) | 1 (12.5%) |
| disease | | | |

Source: Table 14.3.1.5
All TEAEs are coded using MedDRA Version 24.0.
Each participant is counted only once within each system organ class and preferred term.
Percentages are calculated using the number of participants in the corresponding treatment group as the denominator.
Abbreviations: MedDRA = Medical Dictionary for Regulatory Activities; MN = membranous neuropathy; N = number of participants; PT = Preferred Term; SOC = System Organ Class; TEAE = treatment-emergent adverse event.

### Other Adverse Events of Special Interest:

Overall, 7 (87.5%) participants reported an AESI. The most commonly reported AESIs by PT were COVID-19 reported by 3 (37.5%) participants and chills reported by 2 (25.0%) participants. Summary of AESI following budoprutug administration for participants with MN by SOC and PT for the ATP is presented in the Table below.

TABLE
Summary of AESI Following Budoprutug Administration for Participants
with MN by SOC and PT: All Treated Population

| System Organ Class | 100 mg | 200 mg | Total |
|---|---|---|---|
| Preferred Term | (N = 4) | (N = 4) | (N = 8) |
| Number of Participants with an AESI | 3 (75.0%) | 4 (100%) | 7 (87.5%) |
| General disorders and administration | 3 (75.0%) | 1 (25.0%) | 4 (50.0%) |
| site conditions | | | |
| Chills | 1 (25.0%) | 1 (25.0%) | 2 (25.0%) |
| Oedema peripheral | 1 (25.0%) | 0 | 1 (12.5%) |
| Peripheral swelling | 1 (25.0%) | 0 | 1 (12.5%) |
| Infections and infestations | 2 (50.0%) | 2 (50.0%) | 4 (50.0%) |
| COVID-19 | 1 (25.0%) | 2 (50.0%) | 3 (37.5%) |
| Conjunctivitis | 0 | 1 (25.0%) | 1 (12.5%) |
| Pneumonia bacterial | 1 (25.0%) | 0 | 1 (12.5%) |
| Upper respiratory tract infection | 0 | 1 (25.0%) | 1 (12.5%) |
| Musculoskeletal and connective | 2 (50.0%) | 1 (25.0%) | 3 (37.5%) |
| tissue disorders | | | |
| Myalgia | 1 (25.0%) | 0 | 1 (12.5%) |
| Pain in extremity | 1 (25.0%) | 0 | 1 (12.5%) |
| Rhabdomyolysis | 0 | 1 (25.0%) | 1 (12.5%) |
| Investigations | 1 (25.0%) | 1 (25.0%) | 2 (25.0%) |
| Blood creatine phosphokinase | 0 | 1 (25.0%) | 1 (12.5%) |
| increased | | | |
| Blood pressure decreased | 1 (25.0%) | 0 | 1 (12.5%) |
| Nervous system disorders | 2 (50.0%) | 0 | 2 (25.0%) |
| Headache | 1 (25.0%) | 0 | 1 (12.5%) |
| Somnolence | 1 (25.0%) | 0 | 1 (12.5%) |
| Renal and urinary disorders | 1 (25.0%) | 1 (25.0%) | 2 (25.0%) |
| Acute kidney injury | 1 (25.0%) | 0 | 1 (12.5%) |
| Haematuria | 0 | 1 (25.0%) | 1 (12.5%) |
| Haemorrhage urinary tract | 0 | 1 (25.0%) | 1 (12.5%) |
| Respiratory, thoracic and | 0 | 2 (50.0%) | 2 (25.0%) |
| mediastinal disorders | | | |
| Chronic obstructive pulmonary | 0 | 1 (25.0%) | 1 (12.5%) |
| disease | | | |
| Cough | 0 | 1 (25.0%) | 1 (12.5%) |
| Cardiac disorders | 1 (25.0%) | 0 | 1 (12.5%) |
| Sinus tachycardia | 1 (25.0%) | 0 | 1 (12.5%) |
| Gastrointestinal disorders | 0 | 1 (25.0%) | 1 (12.5%) |
| Nausea | 0 | 1 (25.0%) | 1 (12.5%) |
| Vomiting | 0 | 1 (25.0%) | 1 (12.5%) |
| Injury, poisoning and procedural | 1 (25.0%) | 0 | 1 (12.5%) |
| complications | | | |
| Lip injury | 1 (25.0%) | 0 | 1 (12.5%) |
| Psychiatric disorders | 0 | 1 (25.0%) | 1 (12.5%) |
| Depression | 0 | 1 (25.0%) | 1 (12.5%) |
| Skin and subcutaneous tissue | 1 (25.0%) | 0 | (12.5%) |
| disorders | | | |
| Angioedema | 1 (25.0%) | 0 | 1 (12.5%) |
| Vascular disorders | 1 (25.0%) | 0 | 1 (12.5%) |
| Hot flush | 1 (25.0%) | 0 | 1 (12.5%) |

All TEAEs are coded using MedDRA Version 24.0.
Each participant is counted only once within each system organ class and preferred term.
Percentages are calculated using the number of participants in the corresponding treatment group as the denominator.
Abbreviations:
AESI = adverse event of special interest;
MedDRA = Medical Dictionary for Regulatory Activities;
MN = membranous neuropathy;
N = number of participants;
PT = Preferred Term;
SOC = System Organ Class

### Safety Conclusions:

The pattern and incidence of TEAEs were similar between treatment groups.
Overall, 7 (87.5%) participants reported at least one TEAE. Of these, 4 (50.0%) participants had at least 1 treatment-related TEAE.
There were 3 (37.5%) participants with at least one SAE, 1 (12.5%) participant with an ADR, and 3 (37.5%) participants with an AESI. There were no study discontinuations due to an AE.
The most commonly reported SOC was general disorders and administration site conditions reported followed by infections and infestations. The most commonly reported TEAEs by PT were COVID-19 and chills.
No DLTs or deaths were reported during the conduct of this study.
Budoprutug was generally well tolerated and no major safety concerns were observed.

### Discussion and Overall Conclusion:

Overall, 7 (87.5%) participants reported at least one TEAE with 4 (50.0%) participants experiencing at least 1 treatment-related TEAE. Three (3 [37.5%]) participants reported SAEs, 1 (12.5%) participant reported an ADR, and 3 (37.5%) participants reported an AESI during the conduct of this study. Neither of the SAEs were deemed related to the study drug.

The most commonly reported SOC was general disorders and administration site conditions reported followed by infections and infestations. The most commonly reported TEAEs by PT were COVID-19 and chills.

No DLTs or deaths were reported during the conduct of this study.

Following IV infusion of 100 or 200 mg (cumulative 200 mg or 400 mg) of budoprutug to adult participants with primary membranous nephropathy the exposure appeared less than proportional upon doubling the dose. The serum half-life was considered long at 405.80 h and 219.97 h following dosing of 100 mg and 200 mg budoprutug, respectively after the Day 15 dose. The serum clearance was considered low at <0.99 L/day. No considerable accumulation (as determined by Cₘₐₓ) was observed following dosing at 100 mg and 200 mg budoprutug (0.90 and 1.10 respectively).

Mean (SD) percentage change from baseline at the EOS was-78.4 (22.3; −74.6 [26.3] for participants in budoprutug 100 mg group and −84.2 [22.3] for participants in budoprutug 200 mg group). The proportion (95% CI) of participants achieving ≥90% B-cell depletion demonstrated a sharp decrease towards the middle of the study (proportion of 1 [0.566, 1] during Visits 8, 11, 12; 1 [0.510, 1] during visit 13; 1 [0.439, 1] during visit 14), and was then demonstrated to increase again, with a final proportion of 0.4 (0.118, 0.769) at EOS/ET.

The proportion (95% CI) of participants achieving complete remission of proteinuria (UPCR≤0.3 g/g) was 1 (0.342, 1). The proportion (95% CI) of participants achieving ≥50% reduction of proteinuria from baseline was 1 (0.342, 1).

The proportion (95% CI; sensitivity analysis) of participants with NS (with and without edema) resolving on-study was 1 (0.207, 1) in the budoprutug 200 mg group.

Mean (SD) change in serum IgA, IgG, and IgM from baseline over time were-56.8 mg/dL (42.19), 154.2 mg/dL (114.84), and −13.6 mg/dL (5.98), respectively.

Mean (SD) change from baseline in anti-PLA2R antibody levels at EOS was −62.1 RU/mL (24.9). The proportion (95% CI) of anti-PLA2R negative participants was 0.7 (0.2, 0.9) at the end of the study. The proportion (95% CI) of participants achieving a ≥50% and a ≥90% reduction in anti-PLA2R antibodies were 1 (0.4, 1) and 0.7 (0.2, 0.9), respectively.

Budoprutug was generally well tolerated and no major safety concerns were observed. The pattern and incidence of TEAEs were similar between treatment groups.

Overall, there was a considerable amount of variability observed in the determined PK parameters across 2 doses and dosing periods.

A sustained decrease in absolute peripheral CD20+ B-Cell, proteinuria as measured by UPCR, serum IgA and IgM, and anti-PLA2R antibodies was observed from baseline for both treatment groups. All participants achieved complete remission (proteinuria≤0.3 g/g).

### Summary of Phase Ib Trial:

Introduction: Membranous nephropathy (MN) is characterized by a histologic pattern of injury caused by autoantibodies directed against podocyte antigens. Targeting B lymphocytes with anti-CD20 monoclonal antibodies has become a preferred therapy for primary MN, but this strategy fails to induce complete remission (CR) in most patients. Budoprutug is an anti-CD19 monoclonal antibody with a broader spectrum of B-cell depletion, including activity against CD19-positive plasmablasts. Here, we present the results of a Phase 1b, open label, dose escalation study of budoprutug in patients with primary MN.

Methods: Eligible patients had primary MN with a history of nephrotic syndrome, received maximally tolerated therapy with a renin-angiotensin system inhibitor for 6 months, and had a urine protein to creatinine ratio (UPCR) of >2 g/g on two measurements during screening. Budoprutug was administered as 2 bi-weekly doses of 100 or 200 mg six months apart on Days 1,15,169, and 183. The primary objective was safety. Secondary objectives included B-cell levels, anti-phospholipase A2 receptor (anti-PLA2R) antibody (Ab) levels, and changes in proteinuria. The analysis was restricted to patients with at least 48 weeks of follow-up.

Results: Five patients met inclusion criteria, 3 of which were PLA2R Ab positive. All patients achieved complete B cell depletion, with 2 patients reconstituting B-cells before Week 24. Serologic remission was achieved in all anti-PLA2R Ab positive patients over a range of 5 to 18 weeks. Median baseline proteinuria was 4.2 g/g. Three patients achieved complete remission (UPCR≤0.3 g/g, range, 40 to 48 weeks), with a fourth patient achieving a UPCR of 0.33 at week 48. All patients achieved a UPCR<1 g/g and partial remission. Two patients with 72 week follow up achieved durable CRs~18 months from initial dosing. Budoprutug was well tolerated with no serious adverse events or Grade≥3 drug-related AEs.

Conclusions: Treatment with budoprutug resulted in high rates of serologic and clinical remission, suggesting that CD19-targeted B-cell depletion may be a promising approach to the treatment of primary MN. These findings warrant larger studies to confirm the efficacy of budoprutug in MN.

Phase 2 Study: a Phase 2. Open-Label Study to Evaluate the Safety and Efficacy of Budoprutug in Subjects with Primary Membranous Nephropathy

Number of subjects planned: Up to 45 subjects

| Objectives | Endpoints |
|---|---|
| Primary | Primary |
| Evaluate the safety and | Incidence of treatment-emergent adverse |
| tolerability of 3 dose | events (TEAEs) from Baseline through |
| regimens of budoprutug in | Week 48 |
| subjects with PMN | |
| Secondary | Secondary |
| Evaluate the | Absolute and percentage change from |
| pharmacodynamics (PD) of | Baseline in the total number of peripheral |
| 3 dose regimens of | B cells over time |
| budoprutug, assessed as | Change from Baseline in anti-PLA2R |
| decline in and recovery | antibody titers over time |
| of circulating B | |
| cells and anti-PLA2R | |
| autoantibodies | |
| Evaluate the preliminary | Proportion of subjects in each dose cohort |
| clinical efficacy of 3 dose | who achieve complete remission at Week 48 |
| regimens of budoprutug | Proportion of subjects in each dose cohort |
| | who achieve complete or partial remission at |
| | Week 48 |
| | Change from Baseline in proteinuria |
| | (measured via urine protein-creatinine ratio, |
| | UPCR) over time |
| | Change from Baseline in urine albumin-to- |
| | creatinine ratio (UACR) over time |
| | Change from Baseline in eGFR over time |
| | (using the 2021 Chronic Kidney Disease |
| | Epidemiology Collaboration [CKD-EPI] |
| | Creatinine-Cystatin C equation |
| Characterize the | PK parameters, including maximum |
| pharmacokinetics (PK) and | observed concentration (Cmax), area under |
| PK/PD (dose relationship) | the concentration-time curve (AUC), time to |
| of budoprutug in subjects | maximum observed concentration (Tmax), |
| with PMN | terminal half-life (t½), apparent clearance |
| | (CL), and volume of distribution (Vd) |

### Methodology:

This Phase 2, open-label study evaluates the safety and efficacy of 3 dose regimens of budoprutug, as outlined below:

| Dose (mg) | Frequency |
|---|---|
| 200 mg | Cycle 1: Baseline/Day 1 and Day 15 |
| | Cycle 2: Days 169 and 183 |
| 600 mg | Cycle 1: Baseline/Day 1 and Day 15 |
| | Cycle 2: Days 169 and 183 |
| 1000 mg | Cycle 1: Baseline/Day 1 and Day 15 |
| | Cycle 2: Days 169 and 183 |

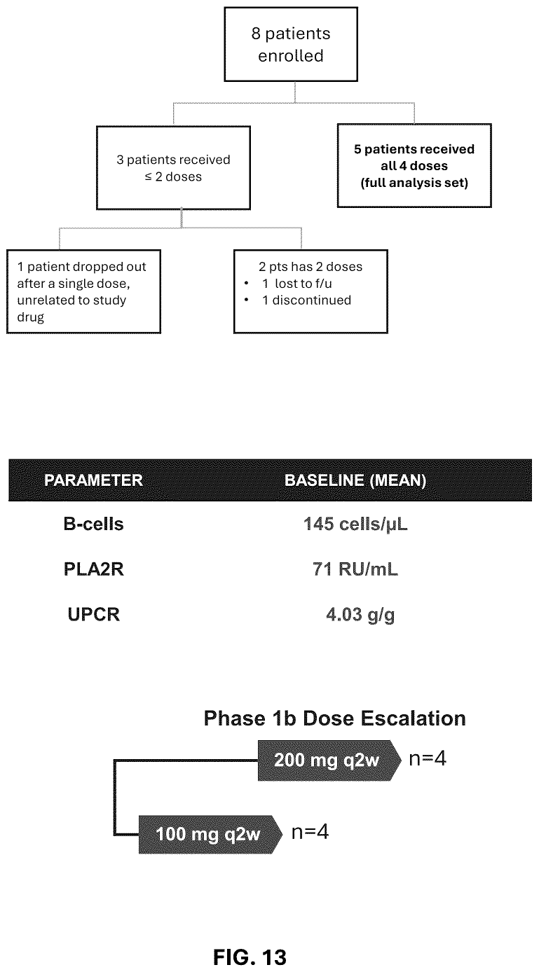

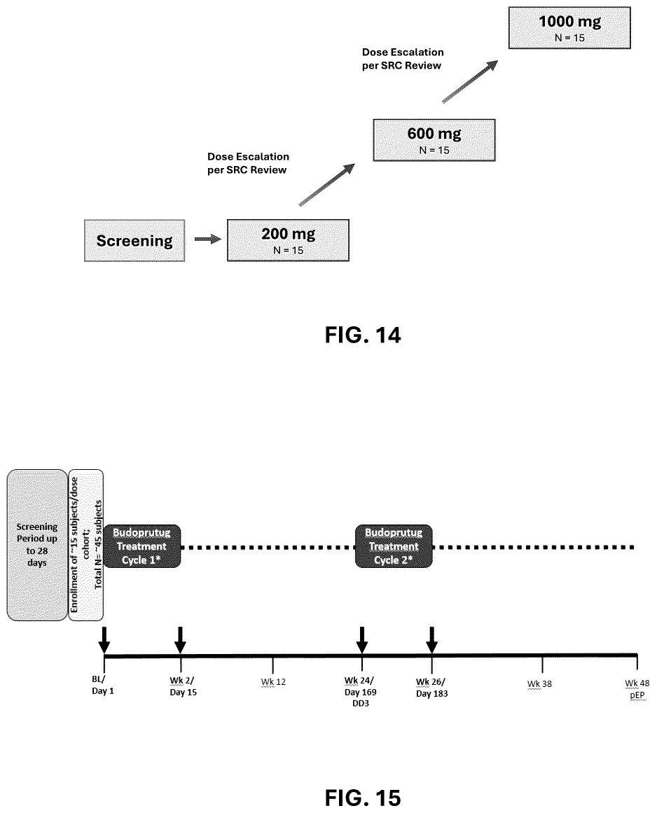

All subjects will be administered 2 cycles of treatment with budoprutug IV infusions. In Cycle 1, the infusions will occur at Baseline (Day 1) and at Week 2 (Day 15). In Cycle 2, the infusions will be administered at Week 24 (Day 169) and Week 26 (Day 183). At each dose-administration visit, subjects will remain at the study site for at least 4 hours after the infusion is completed to monitor for AEs and for blood sample collections for PK and laboratory assessments. Subjects will return at prespecified intervals for assessment of safety, PK and PD. All subjects will be followed through Week 48. Subjects who have not repopulated B cells by Week 48 will return to the clinic for safety and PD assessments until B cell levels return to the subject's Baseline value or are within normal limits. FIG. 14 and FIG. 15 provide an overview of the Phase 2 trial.

The subjects are enrolled sequentially into one of the 3 dose cohorts (budoprutug 200 mg, 600 mg, and 1000 mg). After at least 6 subjects in a cohort continue for at least 8 weeks of treatment and assessments in this study, the Safety Review Committee will meet and review all available data to decide on dose escalation. In FIG. 15, the down arrow indicates the budoprutug dose administration days. The primary endpoint may be incidence of treatment-emergent adverse events (TEAEs).

Study Duration: The study duration for each individual subject in this Phase 2 study will be up to 52 weeks, including up to 4 weeks for screening and a 48-week open-label treatment period.

Study Population: This study will enroll adult subjects who meet all the inclusion criteria and none of the exclusion criteria. Subjects will be eligible for enrollment in this study regardless of race/ethnicity.

Inclusion Criteria: Subjects who meet all the following criteria will be eligible for enrollment.
1. Willing and able to provide written informed consent
2. Aged ≥18 years to ≤70 years old at the time of informed consent
3. Subject has CD19+B cell count≥40 cells/μL at Screening and Baseline
4. Confirmed diagnosis of PMN (e.g., based on a kidney biopsy result, a positive serum anti-PLA2R antibody titer).
5. Subjects must have positive anti-PLA2R antibody titer levels at Screening
6. Subjects with persistent evidence of disease activity despite an adequate trial of ACEi/ARB treatment at maximum tolerated doses, with a stable dose for at least 4 weeks prior to Screening, and no planned or anticipated dose adjustment during the study
7. UPCR≥2.0 g/g during 2 consecutive urine sample collections obtained within 14 days during the Screening period
8. Subjects must have an estimated glomerular filtration rate (eGFR)≥40 mL/min/1.73 m² (by 2021 CKD-EPI creatinine equation)
9. Blood pressure below 160/100 mmHg at Screening and below 150/90 mmHg at Baseline
10. Adequate hematologic, hepatic, and renal function within the Screening period and prior to the first dose of study drug.
11. Willingness to use effective contraception.
   Exclusion Criteria: Subjects who meet any of the following criteria will be excluded from participation in the study:
1. Rapidly progressive glomerulonephritis, or other concomitant or overlapping glomerulopathies based on previous renal biopsy, or glomerulopathies known by the subject's medical history (e.g., diabetic nephropathy, IgA nephropathy, or primary or genetic variant of focal segmental glomerulosclerosis [FSGS])
2. Secondary causes of MN (e.g., malignancy, hepatitis B or C, human immunodeficiency virus [HIV], systemic lupus erythematosus [SLE], or other autoimmune diseases [e.g., thyroiditis, sarcoidosis], or drug-induced). (Note: Potential secondary causes of MN must be ruled out by the investigator, following local guidelines and standard of care.)
3. Prior treatment with any other B cell depleting agent (e.g., rituximab, ocrelizumab, obinutuzumab, ofatumumab, inebilizumab) within 24 weeks prior to the first dose of study drug
4. Prior treatment with corticosteroids or mycophenolate mofetil within 30 days prior to Screening; permitted exceptions include topical corticosteroids, inhaled corticosteroids, or low dose oral corticosteroids (10 mg or less prednisone or equivalent) for non-renal conditions.
5. Treatment with any investigational drug within 30 days or at least 5 times the elimination half-life of the drug (whichever is longer) prior to the first dose of study drug, or subject is currently enrolled in another clinical study or plans to use an investigational device or drug during the
6. Pregnant or lactating females or females who are considering becoming pregnant during the study and for 6 months after the last administration of study drug.
7. History of malignancy (active or suspected) within the past 5 years, except for non-melanoma skin cancer or cervical carcinoma in-situ.
8. Acute, chronic, or latent infection, including SARS-COV-2, hepatitis B virus (HBV), hepatitis C virus (HCV), HIV, or splenectomy that predisposes subject to infections.
9. Subjects with active tuberculosis (TB) or considered at high risk for active TB.
10. Active systemic infection (except common cold) within 4 weeks prior to the Screening visit that requires treatment with anti-infectives, hospitalization, or represents additional risk to the subject as assessed by the investigator.
11. Recent hospitalization for any reason within 14 days prior to Screening, unless approved by the Medical Monitor.
12. Major surgery (e.g., hip replacement, aneurysm removal, stomach ligation) performed within 12 weeks prior to the Screening visit or planned during the conduct of the study.
13. Any other clinically significant disease, condition, or medical history that, in the Investigator's opinion, would compromise safety, interfere with study evaluations or procedures, or make subject unsuitable candidates for the study drug. This includes individuals who are permanently wheelchair-bound, bedridden, or have a very poor functional status that prevents self-care.

Dosage Form and route of administration: Budoprutug drug product will be provided as a colorless to slightly opalescent for administration as an IV infusion.

The table below provides the sequences for nucleic acids, proteins, and peptides discussed herein.

| SEQ ID | |
|---|---|
| NO: | Sequence |
| 1 | CAGGTGCAACTGCAGCAGCCTGGGGCTGAAGTGGTGAAGCCTGGGGCTT |
| | CAGTGAGACTGTCCTGCAAGACTTCTGGCTACACCTTCACCAGCAACTG |
| | GATGCACTGGGTGAAGCAGAGGCCTGGACAAGGCCTTGAGTGGATCGG |
| | AGAGATTGATCCTTCTGATAGTTATACTAACTACAATCAAAAGTTCAAG |
| | GGCAAGGCCAAGTTGACTGTAGACAAATCCTCCAGCACAGCCTACATGG |
| | AAGTCAGCAGCCTGACATCTGAGGACTCTGCGGTCTATTACTGTGCAAG |
| | AGGTAGCAACCCTTACTACTATGCTATGGACTACTGGGGTCAAGGAACC |
| | TCAGTCACCGTCTCCTCA |
| 2 | CAGGTGCAACTGCAGCAGCCTGGGGCTGAAGTGGTGAAGCCTGGGGCTT |
| | CAGTGAGACTGTCCTGCGAGACTTCTGGCTACACCTTCACCAGCAACTG |
| | GATGCACTGGGTGAAGCAGAGGCCTGACCAAGGACTTGAGTGGATCGG |
| | AGAGATTGATCCTTCTGATAGTTATACTAACTACAATCAAAAGTTCAAG |
| | GGCAAGGCCGAATTGACTGTAGACAAATCCTCCAGCACAGCCTACATGG |
| | AAGTCAGCGACCTGACATCTGAGGACTCTGCGGTCTATTACTGTGCAAG |
| | AGGTAGCAACCCTTACTACTATGCTATGGACTACTGGGGTCAAGGAACC |
| | TCAGTCACCGTCTCCTCA |
| 3 | CAGGTGCAACTGCAGCAGCCTGGGGCTGAAGTGGTGAAGCCTGGGGCTT |
| | CAGTGAGACTGTCCTGCAAGACTTCTGGCTACACCTTCACCAGCAACTG |
| | GATGCACTGGGTGAAGCAGAGACCTGGACAAGGACTTGAGTGGATCGG |
| | AGAGATTGATCCTTCTGATAGTTATACTAACTACAATCAAAAGTTCAAG |
| | GGCAAGGCCGAATTGACTGTAGACAAATCCTCCAGCACAGCCTACATGG |
| | AAGTCAGCGACCTGACATCTGAGGACTCTGCGGTCTATTACTGTGCAAG |
| | AGGTAGCAACCCTTACTACTATGCTATGGACTACTGGGGTCAAGGAACC |
| | TCAGTCACCGTCTCCTCA |
| 4 | CAGGTGCAACTGGAGCAGCCTGGGGCTGAAGTGAAGAAGCCTGGGGCT |
| | TCAGTGAAGGTGTCCTGCAAGGCTTCTGGCTACACCTTCACCAGCAACT |
| | GGATGCACTGGGTGAAGCAGAGGCCTGGACAAGGACTTGAGTGGATCG |
| | GAGAGATTGATCCTTCTGATAGTTATACTAACTACAATCAAAAGTTCAA |
| | GGGCAAGGCCAAGTTGACTGTAGACAAATCCTCCAGCACAGCCTACATG |
| | GAAGTCAGCGACCTGACAGCTGAGGACTCTGCGGTCTATTACTGTGCAA |
| | GAGGTAGCAACCCTTACTACTATGCTATGGACTACTGGGGTCAAGGAAC |
| | CTCAGTCACCGTCTCCTCA |
| 5 | CAGGTGCAACTGGAGCAGCCTGGGGCTGAAGTGGTGAAGCCTGGGGCTT |
| | CAGTGAAGGTGTCCTGCAAGACTTCTGGCTACACCTTCACCAGCAACTG |
| | GATGCACTGGGTGAAGCAGACGCCTGGAAAAGGACTTGAGTGGATCGG |
| | AGAGATTGATCCTTCTGATAGTTATACTAACTACAATCAAAAGTTCGAT |
| | GGCAAGGCCAAGTTGACTGTAGACAAATCCTCCAGCACAGCCTACATGG |
| | AAGTCAGCGACCTGACAGCTGAGGACTCTGCGACCTATTACTGTGCAAG |
| | AGGTAGCAACCCTTACTACTATGCTATGGACTACTGGGGTCAAGGAACC |
| | TCAGTCACCGTCTCCTCA |
| 6 | CAGGTGCAACTGGAGCAGCCTGGGGCTGAAGTGAAGAAGCCTGGGGCT |
| | TCAGTGAAGGTGTCCTGCAAGGCTTCTGGCTACACCTTCACCAGCAACT |
| | GGATGCACTGGGTGAGACAGGCACCTGGAAAAGGACTTGAGTGGATCG |
| | GAGAGATTGATCCTTCTGATAGTTATACTAACTACAATCAAAAGTTCGA |
| | TGGCAAGGCCAAGTTGACTGTAGACAAATCCTCCAGCACAGCCTACATG |
| | GAAGTCAGCGACCTGACATCTGAGGACTCTGCGACCTATTACTGTGCAA |
| | GAGGTAGCAACCCTTACTACTATGCTATGGACTACTGGGGTCAAGGAAC |
| | CTCAGTCACCGTCTCCTCA |
| 7 | CAGGTGCAACTGGAGCAGCCTGGGGCTGAAGTGGTGAAGCCTGGGGCTT |
| | CAGTGAAGGTGTCCTGCAAGACTTCTGGCTACACCTTCACCAGCAACTG |
| | GATGCACTGGGTGAAGCAGAGGCCTGGACAAGGACTTGAGTGGATCGG |
| | AGAGATTGATCCTTCTGATAGTTATACTAACTACAATCAAAAGTTCGAT |
| | GGCAAGGCCAAGTTGACTGTAGACAAATCCTCCAGCACAGCCTACATGG |
| | AAGTCAGCGACCTGACATCTGAGGACTCTGCGACCTATTACTGTGCAAG |
| | AGGTAGCAACCCTTACTACTATGCTATGGACTACTGGGGTCAAGGAACC |
| | TCAGTCACCGTCTCCTCA |
| 8 | CAAATTGTTCTCACCCAGTCTCCAGCAATCATGTCTGCATCTCCAGGGGA |
| | GAAGGTCACCATGACCTGCAGTGCCAGCTCAGGTGTCAACTACATGCAC |
| | TGGTACCAGCAGAAGCCAGGCACCTCCCCCAAAAGATGGATTTATGACA |
| | CATCCAAACTGGCTTCTGGAGTCCCTGCTCGCTTCAGTGGCAGTGGGTCT |
| | GGGACCTCTTATTCTCTCACAATCAGCAGCATGGAGGCTGAAGATGCTG |
| | CCACTTATTACTGCCATCAGCGAGGTAGTTACACGTTCGGAGGGGGGAC |
| | CAAGCTGGAAATAAA |
| 9 | CAAATTGCTCTCACCCAGGAGCCAGCAATCATGTCTGCATCTCCAGGGG |
| | AGAAGGTCACCATGACCTGCAGTGCCAGCTCAGGTGTCAACTACATGCA |
| | CTGGTATCAGCAGAAGCCAGGCACCTCCCCCAAAAGATGGATTTATGAC |
| | ACATCCAAACTGGATTCTGGAGTCCCTGCTCGCTTCAGTGGCAGTGGGT |
| | CTGGGACCTCTTATTCTCTCACAATCAGCAGCATGGAGGCTGAAGATGC |
| | TGCCACTTATTACTGCCATCAGCGAGGTAGTTACACGTTCGGAGGGGGG |
| | ACCAAGCTGGAAATAAA |
| 10 | GACATTGTTCTCACCCAGTCTCCAGCAACTTTGTCTGCATCTCCAGGGGA |
| | GAAGGTCACCATGACCTGTAGTGCCAGCTCAGGTGTCAACTACATGCAC |
| | TGGTATCAGCAGAAGCCAGGCACCTCCCCCAAAAGATGGATTTATGACA |
| | CATCCAAACTGGATTCTGGAGTCCCTGCTCGCTTCAGTGGCAGTGGGTCT |
| | GGGACCTCTTATTCTCTCACAATCAGCAGCATGGAGGCTGAAGATGCTG |
| | CCACTTATTACTGCCATCAGCGAGGTAGTTACACGTTCGGAGGGGGGAC |
| | CAAGCTGGAAATAAA |
| 11 | CAAATTGTTCTCACCCAGTCTCCAGCAACTTTGTCTGCATCTCCAGGGGA |
| | GAAGGCTACCATGACCTGCAGTGCCAGCTCAGGTGCTAACTACATGCAC |
| | TGGTACCAGCAGAAGCCAGGCACCTCCCCCAAAAGATGGATTTATGACA |
| | CATCCAAACTGGCTTCTGGAGTCCCTGCTCGCTTCAGTGGCAGTGGGTCT |
| | GGGACCTCTTATTCTCTCACAATCGAGAGCATGGAGGCTGAAGATGCTG |
| | CCACTTATTACTGCCATCAGCGAGGTAGTTACACGTTCGGAGGGGGGAC |
| | CAAGCTGGAAATAAA |
| 12 | CAAATTGTTCTCACCCAGTCTCCAGCAACTTTGTCTGCATCTCCAGGGGA |
| | GAAGGCTACATGACCTGTAGTGCCAGCTCAGGTGTCAACTACATGCACT |
| | GGTACCAGCAGAAGCCAGGCACCTCCCCCAAAAGATGGATTTATGACAC |
| | AGACAAAACGGCTTCTGGAGTCCCTGCTCGCTTCAGTGGCAGTGGGTCT |
| | GGGACCTCTTATTCTCTCACAATCAGCAGCATGGAGGCTGAAGATGCTG |
| | CCACTTATTACTGCCATCAGCGAGGTAGTTACACGTTCGGAGGGGGGAC |
| | CAAGCTGGAAATAAA |
| 13 | QVQLQQPGAEVVKPGASVRLSCKTSGYTFTSNWMHWVKQRPGQGLEWIG |
| | EIDPSDSYTNYNQKFKGKAKLTVDKSSSTAYMEVSSLTSEDSAVYYCARGS |
| | NPYYYAMDYWGQGTSVTVSS |
| 14 | QVQLQQPGAEVVKPGASVRLSCETSGYTFTSNWMHWVKQRPDQGLEWIG |
| | EIDPSDSYTNYNQKFKGKAELTVDKSSSTAYMEVSDLTSEDSAVYYCARGS |
| | NPYYYAMDYWGQGTSVTVSS |
| 15 | QVQLQQPGAEVVKPGASVRLSCKTSGYTFTSNWMHWVKQRPGQGLEWIG |
| | EIDPSDSYTNYNQKFKGKAELTVDKSSSTAYMEVSDLTSEDSAVYYCARGS |
| | NPYYYAMDYWGQGTSVTVSS |
| 16 | QVQLEQPGAEVKKPGASVKVSCKASGYTFTSNWMHWVKQRPGQGLEWIG |
| | EIDPSDSYTNYNQKFKGKAKLTVDKSSSTAYMEVSDLTAEDSAVYYCARGS |
| | NPYYYAMDYWGQGTSVTVSS |
| 17 | QVQLEQPGAEVVKPGASVKVSCKTSGYTFTSNWMHWVKQTPGKGLEWIG |
| | EIDPSDSYTNYNQKFDGKAKLTVDKSSSTAYMEVSDLTAEDSATYYCARGS |
| | NPYYYAMDYWGQGTSVTVSS |
| 18 | QVQLEQPGAEVKKPGASVKVSCKASGYTFTSNWMHWVRQAPGKGLEWIG |
| | EIDPSDSYTNYNQKFDGKAKLTVDKSSSTAYMEVSDLTSEDSATYYCARGS |
| | NPYYYAMDYWGQGTSVTVSS |
| 19 | QVQLEQPGAEVVKPGASVKVSCKTSGYTFTSNWMHWVKQRPGQGLEWIG |
| | EIDPSDSYTNYNQKFDGKAKLTVDKSSSTAYMEVSDLTSEDSATYYCARGS |
| | NPYYYAMDYWGQGTSVTVSS |
| 20 | QVQLQQPGAEVKKPGASVRLSCETSGYTFTSNWMHWVKQRPDQGLEWIG |
| | EIDPSDSYTNYNQKFDGKAELTVDKSSSTAYMEVSDLTSEDSAVYYCARGS |
| | NPYYYAMDYWGQGTSVTVSS |
| 21 | QVQLEQPGAEVKKPGASVKVSCKASGYTFTSNWMHWVKQTPGKGLEWIG |
| | EIDPSDSYTNYNQKFDGKAKLTVDKSSSTAYMEVSDLTAEDSATYYCARGS |
| | NPYYYAMDYWGQGTSVTVSS |
| 22 | QVQLXQPGAEVXKPGASVXXSCXXSGYTF |
| 23 | WVXQXPXXGLEWIG |
| 24 | YNQKFXGKAXLTVDKSSSTAYMEVSXLTXEDSAXYYCA |
| 25 | QIVLTQSPAIMSASPGEKVTMTCSASSGVNYMHWYQQKPGTSPKRWIYDTS |
| | KLASGVPARFSGSGSGTSYSLTISSMEAEDAATYYCHQRGSYTFGGGTKLEI |
| | K |
| 26 | QIALTQEPAIMSASPGEKVTMTCSASSGVNYMHWYQQKPGTSPKRWIYDTS |
| | KLDSGVPARFSGSGSGTSYSLTISSMEAEDAATYYCHQRGSYTFGGGTKLEI |
| | K |
| 27 | DIVLTQSPATLSASPGEKVTMTCSASSGVNYMHWYQQKPGTSPKRWIYDTS |
| | KLDSGVPARFSGSGSGTSYSLTISSMEAEDAATYYCHQRGSYTFGGGTKLEI |
| | K |
| 28 | QIVLTQSPATLSASPGEKATMTCSASSGANYMHWYQQKPGTSPKRWIYDTS |
| | KLASGVPARFSGSGSGTSYSLTIESMEAEDAATYYCHQRGSYTFGGGTKLEI |
| | K |
| 29 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTKL |
| | EIK |
| 30 | QIALTQEPAIMSASPGEKATMTCSASSGVNYMHWYQQKPGTSPKRWIYDTS |
| | KLDSGVPARFSGSGSGTSYSLTIESMEAEDAATYYCHQRGSYTFGGGTKLEI |
| | K |
| 31 | QIVLTQSPATLSASPGEKATMTCSASSGANYMHWYQQKPGTSPKRWIYDT |
| | DKTASGVPARFSGSGSGTSYSLTIESMEAEDAATYYCHQRGSYTFGGGTKL |
| | EIK |
| 32 | XIXLTQXPAXXSASPGEKXTMTC |
| 33 | DTXKXX |
| 34 | QVQLQQPGAEVVKPGASVRLSCKTSGYTFTSNWMHWVKQRPGQGLEWIG |
| | EIDPSDSYTNYNQKFKGKAKLTVDKSSSTAYMEVSSLTSEDSAVYYCARGS |
| | NPYYYAMDYWGQGTSVTVSS |
| 35 | QIVLTQSPAIMSASPGEKVTMTCSASSGVNYMHWYQQKPGTSPKRWIYDTS |
| | KLASGVPARFSGSGSGTSYSLTISSMEAEDAATYYCHQRGSYTFGGGTKLEI |
| | K |
| 36 | VRLSCKTSG |
| 37 | WVKQRPGQG |
| 38 | YNQKFKGKA |
| 39 | FKGKAKLTV |
| 40 | YMEVSSLTS |
| 41 | VYYCARGSN |
| 42 | IVLTQSPAI |
| 43 | VLTQSPAIM |
| 44 | VTMTCSASS |
| 45 | VNYMHWYQQ |
| 46 | WIYDTSKLA |
| 47 | IYDTSKLAS |
| 48 | CGTAAGTGGATCC |
| 49 | GCTAGCTCCAGC |
| 50 | QVQLEQPGAEVVKPGASVKVSCKTSGYTFTSNWMHWVKQTPGKGLEWIG |
| | EIDPSDSYTNYNQKFDGKAKLTVDKSSSTAYMEVSDLTAEDSATYYCARGS |
| | NPYYYAMDYWGQGTSVTVSSASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 51 | QVQLEQPGAEVVKPGASVKVSCKTSGYTFTSNWMHWVKQTPGKGLEWIG |
| | EIDPSDSYTNYNQKFDGKAKLTVDKSSSTAYMEVSDLTAEDSATYYCARGS |
| | NPYYYAMDYWGQGTSVTVSSASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 52 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTK |
| | SFNRGEC |
| 53 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTK |
| | SFNRGEC |
| 54 | QVQLEQPGAEVVKPGASVKVSCKTSGYTFTSNWMHWVKQTPGKGLEWIG |
| | EIDPSDSYTNYNQKFDGKAKLTVDKSSSTAYMEVSDLTAEDSATYYCARGS |
| | NPYYYAMDYWGQGTSVTVSSASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 55 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTK |
| | SFNRGEC |

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### List of Abbreviations and Definition of Terms

| Abbreviation | Definition |
|---|---|
| ADA | Anti-Drug Antibody |
| ADR | Adverse drug reaction |
| AE | Adverse event |
| AESI | Adverse event of special interest |
| AKI | Acute kidney injury |
| ALT | Alanine aminotransferase |
| AST | Aspartate aminotransferase |
| ATP | All treated population |
| AUC | Area under the plasma concentration-time curve |
| BP | Blood pressure |
| CD | Cluster of Differentiation |
| CDC | Complement-dependent cytotoxicity |
| CI | Confidence interval |
| Cₘₐₓ | Maximum observed plasma drug concentration |
| CNI | Calcineurin inhibitors |
| COPD | Chronic obstructive pulmonary disease |
| CRS | Cytokine release syndrome |
| CTCAE | Common Terminology Criteria for Adverse Events |
| DLT | Dose-limiting toxicity |
| DRC | Data Review Committee |
| ECG | Electrocardiogram |
| eCRF | electronic case report form |
| EDC | Electronic data capture |
| eGFR | Estimated glomerular filtration rate |
| EIU | Exposure In Utero |
| EOS | End of study |
| ESRD | End-stage renal disease |
| ET | Early termination |
| FAS | Full analysis set |
| FDA | Food and Drug Administration |
| FSGS | Focal segmental glomerulosclerosis |
| GCP | Good Clinical Practice |
| GMP | Good Manufacturing Practices |
| HAHA | Human anti-human antibodies |
| HIV | Human immunodeficiency virus |
| ICF | Informed consent form |
| ICH | The International Council for Harmonisation of Technical |
| | Requirements for Pharmaceuticals for Human Use |
| IEC | Independent Ethics Committee |
| Ig | Immunoglobulins |
| IRB | Institutional Review Board |
| IRR | Infusion-related reaction |
| IV | Intravenous |
| KDIGO | Kidney Disease Improving Global Outcomes |
| mAb | Monoclonal antibody |
| MN | Membranous nephropathy |
| NCI | National Cancer Institute |
| NS | Nephrotic syndrome |
| PBMC | Primary peripheral blood mononuclear cells |
| PC | Plasma cell |
| PD | Pharmacodynamic |
| PK | Pharmacokinetic |
| PLA2R | Phospholipase A2 receptor |
| pMN | |
| PROMIS | Patient-Reported Outcomes Measurement Information |
| | System |
| PT | Preferred term |
| QOL | Quality of life |
| RASi | Renin angiotensin system inhibitor |
| SAE | Serious adverse event |
| SAP | Statistical analysis plan |
| SCID | Severe combined immunodeficient |
| SD | Standard deviation |
| SOC | System, organ, class |
| SUSAR | Suspected Unexpected Serious Adverse Reactions |
| TEAE | Treatment-emergent adverse event |
| UACR | Urine albumin-to-creatinine ratio |
| ULN | Upper limit of normal |
| UPCR | Urine protein-to-creatinine ratio |

1. A method of treating a subject having membranous nephropathy (MN), the method comprising: providing a composition comprising a therapeutically effective amount an anti-CD19 antibody to the subject having an anti-phospholipase A2 receptor (PLA2R) antibody, wherein the anti-CD19 antibody comprises: a heavy chain variable region at least 95% identical to SEQ ID NO: 17 and/or a light chain variable region at least 95% identical to SEQ ID NO: 29.

2. The method of claim 1, wherein membranous nephropathy in the subject is primary membranous nephropathy (PMN).

3. The method of claim 1, wherein the anti-CD19 antibody comprises a heavy chain variable region comprising SEQ ID NO: 17 and/or a light chain variable region comprising SEQ ID NO: 29, and preferably said anti-CD19 antibody is budoprutug.

4. The method of claim 3, wherein the composition is administered parenterally.

5. (canceled)

6. (canceled)

7. The method of claim 3, wherein the composition comprises about 100 mg budoprutug.

8. The method of claim 3, wherein the composition comprises about 200 mg budoprutug.

9. The method of claim 3, wherein the composition comprises about 600 mg budoprutug.

10. The method of claim 3, wherein the composition comprises about 1000 mg budoprutug.

11. The method of claim 7, wherein the composition is administered once every two weeks.

12. The method of claim 7, wherein the composition is administered at days 1, 15, 169, and 183.

13. The method of claim 7, wherein the composition is administered in treatment cycles, wherein one treatment cycle comprises administering the composition once every two weeks, with a 24-week interval between two cycles.

14. The method of claim 7, wherein the composition is administered in treatment cycles, each treatment cycle comprising administration of the composition at days 1 and 15, and wherein there is a 24-week interval between each treatment cycle.

15. (canceled)

16. A method for treating a subject having membranous nephropathy (MN), the method comprising:
determining if the subject has an anti-phospholipase A2 receptor (PLA2R) antibody in serum;
administering composition comprising a therapeutically effective amount an anti-CD19 antibody to the subject if the subject has anti-PLA2R antibody in serum;
wherein the anti-CD19 antibody comprises: a heavy chain variable region at least 95% identical to SEQ ID NO: 17 and/or a light chain variable region at least 95% identical to SEQ ID NO: 29.

17. The method of claim 16, wherein membranous nephropathy in the subject is primary membranous nephropathy (PMN).

18. The method of claim 16, wherein the anti-CD19 antibody is budoprutug.

19. The method of claim 18, wherein the composition is administered parenterally.

20. The method of claim 19, wherein the composition is administered via an intravenous (IV) infusion.

21. (canceled)

22. The method of claim 18, wherein the composition comprises about 100 mg budoprutug.

23. The method of claim 18, wherein the composition comprises about 200 mg budoprutug.

24. The method of claim 18, wherein the composition comprises about 600 mg budoprutug.

25.-44. (canceled)
